(19) United States
(12) Patent Application Publication
(10) Pub. No.: US 2016/0145679 A1
(43) Pub. Date: May 26, 2016

(54) OLIGONUCLEOTIDE FUNCTIONALIZED QUANTUM DOTS

(71) Applicant: ARIZONA BOARD OF REGENTS ON BEHALF OF ARIZONZA STATE UNIVERSITY, Scottsdale, AZ (US)

(72) Inventors: Hao YAN, Chandler, AZ (US); Yan LIU, Chandler, AZ (US); Zhengtao DENG, Tempe, AZ (US); Anirban SAMANTA, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 14/764,913

(22) PCT Filed: Feb. 27, 2014
(86) PCT No.: PCT/US14/19094

§ 371 Date: Jul. 30, 2015

Related U.S. Application Data
(60) Provisional application No. 61/771,728, filed on Mar. 1, 2013.

(51) Int. Cl. C12Q 1/68; G01N 33/58

(52) U.S. Cl. CPC C12Q 1/6825; C12Q 1/6834; G01N 33/588; Y10S 977/901; Y10S 977/824; Y10S 977/795; Y10S 977/774; B82Y 15/00

(57) ABSTRACT

The present invention provides a DNA-functionalized conjugate comprising single-stranded DNA (ssDNA) strands conjugated to a semi-conductor nanoparticle, wherein the nanoparticle provides fluorescent emissions. The present invention also provides a method to efficiently conjugate DNA strands to a wide variety of quantum dots having fluorescent emissions

### PRIORITY OF INVENTION

This application claims priority to U.S. Provisional Application Number 61/771,728, filed Mar. 1, 2013. The entire content of this application is hereby incorporated herein by reference.

### GOVERNMENT FUNDING

This invention was made with government support under N00014-09-1-1118 awarded by The Office of Naval Research. The government has certain rights in the invention.

### BACKGROUND OF THE INVENTION

Nanotechnology has been studied in many different research areas and is being applied for the development of scientific and industrial technologies, such as nano electronics, sensors, and catalysts. DNA nanotechnology offers a compelling approach toward programmable nanoparticle patterning. One common strategy to obtain such structures was to first link a single stranded DNA (ssDNA) oligonucleotides to nanoparticle and subsequently uses the sequence information or ssDNA to control the positioning or the DNA modified nanoparticle conjugates onto ssDNA scaffolds through direct complementary base-pairing hybridization. The self-assembly of metallic Au nanoparticles and programmed complex architectures using DNA have been demonstrated one decade ago. DNA-induced self-assembly of nanoparticles was first introduced in 1996 by pioneering papers of Mirkin and co-workers, and Alivisatos and co-workers. The ability of specific hybridization of DNA was utilized for self-assembly of nanoparticles on which ssDNA is chemically immobilized. Followed by these successes, DNA-directed self-assembly has shown great progress in. constructing one-dimensional, two-dimensional, three-dimensional, and even chiral AuNP architectures. However, the self-assembly of semiconductor nanoparticle, such as highly fluorescent colloidal quantum dots (QDs), has greatly fell behind to the Au NPs. This is due to that achieving reliable conjugation of the ssDNA with semiconductor nanoparticles. Although efforts have been devoted to this field, developing a general and reliable method for production of ss-DNA semiconductor nanoparticles is still an open challenging prospect.

### SUMMARY OF THE INVENTION

The present inventors developed a novel method to allow the efficient conjugation of DNA strands to a wide variety of quantum dots with fluorescent emissions covering from UV-vis to IR.

In certain embodiments, the present invention provides a DNA-functionalized conjugate comprising single-stranded DNA (ssDNA) strands conjugated to a semiconductor nanoparticle, wherein the nanoparticle provides fluorescent emissions. In certain embodiments, the emission ranges from UV-vis to IR (360-800nm). In certain embodiments, the nanoparticle is a zero-dimensional quantum dot, a one-dimensional quantum rod or wire, a two-dimensional quantum ribbon or sheet, or a three-dimensional structure. In certain embodiments, the semiconductor nanoparticle comprises a binary material. In certain embodiments, the binary material is CdTe, CdSe, CdS, ZnSe, PbS, PbSe, ternary alloyed ZnCdSe, CdSeS, CdPbTe, quaternary alloyed ZnCdSSe, Mn doped ZnSe or Mn doped ZnS. In certain embodiments, the conjugation size is between 2 nm to 50 nm. In certain embodiments, the DNA is a chimeric DNA comprising a phosphorothiolated phosphorodiester back bone. In certain embodiments, the DNA-functionalized conjugate is stable and highly fluorescent. In certain embodiments, the nanoparticle is a quantum dot comprising a core and a shell. In certain embodiments, the shell material is binary CdS, ZnS, ZnSe, CdSe, or ternary ZnCdS. In certain embodiments, the shell comprises 1-20 monolayers. In certain embodiments, the DNA directly inserts into the shell. In certain embodiments, the conjugate is stable over a pH range of 4-12. In certain embodiments, the conjugate is stable at salt concentrations of greater than 10 nM Na+ or Mg2+. In certain embodiments, the conjugate exhibits bright fluorescence emission with quantum yields of up to 70%.

In certain embodiments, the present invention provides a DNA origami construct comprising the DNA functionalized conjugate described above and a DNA origami structure.

In certain embodiments, the present invention provides a method of synthesizing a DNA-functionalized conjugate, comprising encapsulating a nanoparticle core with a shell in the presence of ssDNA conjugate single-stranded DNA (ssDNA) to form a DNA-functionalized quantum dot.

### BRIEF DESCRIPTION OF THE FIGURES

FIG. 1. DNA functionalization of core/shell QDs and organization by self-assembled DNA origami. Core/shell QDs are functionalized with oligonucleotides during shell growth in aqueous solution at 90° C. for 20-120 min. The resulting core/thick-shell QDs are chemically, photonically, and colloidally stable and display PL quantum yields of up to 70% and broad spectral tunability from the UV to the NIR. The DNA oligonucleotides contain phosphorothiolated (ps) domains (5-10 nucleotides; shown in violet) for incorporation of the DNA directly into the outer QD shells and a typical phosphodiester (po) backbone (blue) for recognition by DNA capture probes within origami structures. The QD core (red) can be synthesized in aqueous solution or an organic solvent. The shells (green) are CdS or ZnS. Simultaneously, self assembled DNA origami structures were synthesized by thermal annealing of M13 DNA with staple strands (gray) and capture strands (red). Finally, hybridization of the recognition domains of the QD—DNA conjugates to complementary capture strands displayed on the surface of the DNA origami yields higher-order architectures.

FIG. 2. DNA-functionalized CdTe/7CdS core/shell QDs with MPA capped magic-sized CdTe cores and emission at 672 nm. (a) TEM and HRTEM images of the QD—DNA conjugates. (b) UVvis absorption and PL emission spectra of the conjugates (red) and the reference dye rhodamine 101 (black) for PL quantum yield measurements. Comparison with the PL intensity of the reference dye revealed that the QD—DNA conjugates displayed a quantum yield of 70%. (c) Photographs of the QDs illuminated with a 365 nm UV lamp in several different buffers: (1) 1×TAEMg²⁺; (2) 10x TAE Mg²⁺; (3) 1×TBE Mg²⁺; (4-6) PBS buffers at pH 4,7, and 10, respectively. (dg) AFM images and height profiles of the CdTe/CdS QDs organized by triangular (three QDs total, one QD per arm) and rectangular (two QDs total, in opposite corners) DNA origami. The inset in (d) is a STEM image of CdSe/7CdS QD—DNA conjugates assembled on triangular DNA origami, whose triangular shape was made visible by negative staining with uranyl formate.

FIG. 3. Characterization of various stages during the synthesis of CdSe/20CdS QD—DNA conjugates. (ac) TEM and (insets) STEM images and (df) HRTEM images of (a, d) OLA-capped CdSe core QDs (spherical, 6 nm in diameter), (b, e) core/shell CdSe/9CdS QDs (tetrahedral, 12 nm in length), and (c, f) thick-shell CdSe/20CdS QD—DNA conjugates (tetrahedral, 18 nm in length). (g) AFM image and (inset) height profile and (i) STEM image of CdTe/20CdS QD—DNA conjugates organized by triangular DNA origami. (h) PL and (j) EDS spectra of CdSe QDs (black, “1”), CdSe/9CdS QDs (red, “2”) and CdTe/20CdS QD—DNA conjugates (wine, “3”).

FIG. 4. (a) Fluorescence spectra of a series of DNA-functionalized core/shell QDs with tunable emission from the UV to the NIR. (b) Zoomed-out and -in AFM images and corresponding height profiles of QD—DNA/DNA origami structures. The conjugates have the following emissions and compositions: UV-emitting (360 nm) ZnSe/4ZnS QDs; blue-emitting (425 nm) ZnSe/4ZnS QDs; green-emitting (510 nm) ZnCdSSe/4ZnS QDs; yellow-emitting (555 nm) CdTe/2ZnS QDs; yellow-emitting (575 nm) CdTe/2CdS; orange-emitting (610 nm) CdTe/4CdS; NIR-emitting (740 nm) CdTe/8CdS; and NIR-emitting (800 nm) CdTe/13CdS, respectively. The scale bars are 100 nm.

FIG. S1. Quantum yield measurement of oligonucleotide functionalized CdTe/7 CdS core/shell QDs using purified CdTe core. (a) UV-Vis absorption and (b) photoluminescence emission spectra of QDs and the standard Rhodamine 101.

FIG. S2. Quantum yield measurement of oligonucleotide functionalized CdTe/7 CdS core/shell QDs using unpurified CdTe core. (a) UV-Vis absorption and (b) photoluminescence emission spectra of QDs and the standard Rhodamine 101.

FIG. S3. (a) TEM and (b) HRTEM images of the oligonucleotide functionalized CdTe/7CdS core/shell QDs with emission at 672 nm using purified CdTe core.

FIG. S4. TEM and HRTEM images of the oligonucleotide functionalized CdTe/13CdS core/shell QDs with emission at 800 nm using purified CdTe core.

FIG. S5. Additional STEM image of the self-assembled QDs on DNA origami and EDS pattern of the oligonucleotide functionalized CdTe/7CdS QDs measured from the sample shown in the STEM image. Note that the phosphorus signal is derived from the DNA, and the uranium is derived from the negative stain.

FIG. S6. Additional AFM images of the oligonucleotide functionalized CdTe/2CdS core/shell QDs (emission at 575 nm) organized by DNA origami. Nearly 100% yield of QD assembly on the origami was obtained.

FIG. S7. Additional AFM images of the oligonucleotide functionalized CdTe/7CdS core/shell QDs self-assembled on DNA origami. Nearly 100% yield of QD assembly on the origami was obtained.

FIG. S8. Evidence of the DNA being ‘embedded’ within the QD shell. The PL spectra of the oligonucleotide functionalized CdTe/5CdS core/shell QDs before and after etching using citrate buffer (pH=3, 5 minute exposure). The PL emission is shifted from 625 nm to 585 nm as shown in (a), indicating etching of the surface layer. Next, the sample was purified by a 0.5 mL Amicon filter (MWCO 301(13a). The purified QDs were attached to DNA origami with a yield of 60% as shown in (b-c), indicating that the oligonucleotides were actually ‘nailed’ into the CdS shell.

FIG. S9. UV-Vis and PL spectra of the 3.0 nm CdSe core QDs (565 nm emission, green trace) and the CdSe/9 CdS core/shell QDs conjugated to DNA (635 nm emission, red trace). Note that the PL shift to a red wavelength is due to the formation of the thick-shell.

FIG. S10. UV-Vis absorption and PL spectra of the 6.0 nm core CdSe QDs (black), CdSe/9CdS (red), and the CdSe/20 CdS QDs (maroon). Note that the PL shift about 13 nm after coating by the thick-shell.

FIG. S11. (a) TEM, (b) STEM and (c-d) HRTEM images of the 6.0 nm CdSe core QDs with emission at 650 nm.

FIG. S12. TEM (a, c, e) and STEM (b, d, f) images of the CdSe/9CdS core/shell QDs with 6.0 nm CdSe core.

FIG. S13. Additional TEM images of the oligonucleotides functionalized thick-shell CdTe/20 CdS QDs with 6.0 nm CdSe core.

FIG. S14. Enlarged HRTEM image of the oligonucleotide functionalized thick-shell CdTe/20 CdS QDs with 6.0 nm CdSe core. Note that the thick-shell QDs are well-crystallized. The blue box indicates the edges of a single nanoparticle.

FIG. S15. Enlarged STEM image of the oligonucleotide functionalized thick-shell CdTe/20 CdS QDs with 6.0 nm CdSe core.

FIG. S16. Additional STEM image of the QDs assembled on DNA origami and EDS pattern of the oligonucleotide functionalized CdSe/20 CdS QDs with 6.0 nm CdSe core measured from the sample shown in the STEM image. Note that the phosphorus signal is derived from the DNA, and the uranium signal is derived from the negative stain.

FIG. S17. Additional AFM image, typical HRTEM, and STEM image of the oligonucleotide functionalized CdTe/4ZnS QDs on DNA origami. Note that the red arrows are other DNA impurities.

FIG. S18. (a) Additional AFM image, (b) STEM image and (c) EDS pattern of the oligonucleotide functionalized ZnSe/4ZnS QDs on DNA origami. Note that in the EDS spectra, the phosphorus signal is derived from the DNA, and the uranium signal is derived from the negative stain.

FIG. S19. (a) Additional AFM image and (b) STEM image of the oligonucleotide functionalized CdS/4ZnS QDs on DNA origami.

FIG. S20. UV-Vis and PL spectra of the Zn_{0.60}Cd_{0.40}S_{0.33}Se_{0.66} core QDs and oligonucleotide functionalized Zn_{0.60}Cd_{0.40}S_{0.33}Se_{0.66}/4 ZnS core/shell QDs.

FIG. S21. AFM image, STEM image and EDS spectrum of the oligonucleotide functionalized Zn_{0.60}Cd_{0.40}S_{0.33}Se_{0.66}/4 ZnS QDs on DNA origami. Note that the phosphorus signal is derived from the DNA, and the uranium signal is derived from the negative stain.

FIG. S22. Design of the triangular DNA origami.

FIG. S23. Design of the rectangular DNA origami.

### DETAILED DESCRIPTION

The invention provides a new method for conjugation of single stranded DNA to semiconductor nanoparticles, such as zero-dimensional (OD) quantum dots or one-dimensional (1 D) quantum rods or quantum wires, or two-dimensional (2D) quantum ribbons or sheets with highly tunable and bright photoluminescence (quantum yield up to 65%) spanning from UV to IR (350 to 3000 nm). The materials of the core semiconductor nanoparticles could be binary such as, CdTe, CdSe, CdS, ZnSe, PbS, PbSe, ternary alloyed ZnCdSe, CdSeS, CdPbTe, quaternary alloyed ZnCdSSe, Mn doped ZnSe or Mn doped ZnS, and the shell materials could be binary CdS, ZnS, ZnSe, CdSe, or ternary ZnCdS. The core size and shell thickness could be finely tuned, and formed core/shell quantum heterostructure could be either Type I, quasi type II, or Type II configuration. The overall size the conjugation could be tuned between 2 nm to 50 nm. The length and bases in the DNA sequence could be attributably selected. The method takes advantage of a phosphorothiolated back bone modified ps-po-chimeric DNA strands that could directly insert into the quantum dot shell during the core/shell nanostructure formation, resulting in stable and highly fluorescent semiconductor nanoparticles-ssDNA conjugates. These ssDNA conjugated semiconductor nanoparticles could be programmable positioned on addressable DNA origami through direct complementary base-pairing hybridization, which are revealed by Atomic force microscopy (AFM) and Scanning Transmission electron microscopy (STEM) imaging techniques. Our invention opens up new opportunities to construct muticomponent discrete semiconductor or semiconductor-metal hybrid nanostructures for energy, nanophotonics, and biosensing applications. Frontier energy, medical, and security-related nanotechnology will depend on the integration and optimization of semiconductor nanoparticle-based technologies and biological sciences to design hybrid materials for increasingly cleaner and more efficient energy conversion and storage, as well as biological sensors having increased sensitivity.

The assembly and isolation of DNA oligonucleotide-functionalized gold nanoparticles (AuNPs) has become a well-developed technology that is based on the strong bonding interactions between gold and thiolated DNA. However, achieving DNA-functionalized semiconductor quantum dots (QDs) that are robust enough to withstand precipitation at high temperature and ionic strength through simple attachment of modified DNA to the QD surface remains a challenge. We report the synthesis of stable core/shell (1-20 monolayers) QD—DNA conjugates in which the end of the phosphorothiolated oligonucleotide (5-10 nucleotides) is “embedded” within the shell of the QD. These reliable QD—DNA conjugates exhibit excellent chemical and photonic stability, colloidal stability over a wide pH range (4-12) and at high salt concentrations (>100 mM Na⁺ or Mg²⁺), bright fluorescence emission with quantum yields of up to 70%, and broad spectral tunability with emission ranging from the UV to the NIR (360-800 nm).

Organizing inorganic nanoparticles (NPs) with nanoscale precision is of great interest for energy, nanophotonics, and nanobiotechnology applications. ((a) Service, R. F. Science 2005, 309, 95. (b) Srivastava, S.; Santos, A.; Critchley, K.; Kim, K. S.; Podsiadlo, P.; Sun, K.; Lee, J.; Xu, C. L.; Lilly, G. D.; Glotzer, S. C.; Kotov, N. A. Science 2010, 327, 1355. (c) Liu, Y. Nat. Nanotechnol. 2011, 6, 463.) One of the most promising approaches for the fully programmable self-assembly of NPs, DNA nanotechnology, relies on Watson-Crick basepairing interactions between DNA-functionalized NPs and underlying DNA nanoscaffolds. ((2) (a) Seeman, N. C. Nature 2003, 421, 427. (b) Pinheiro, A. V.; Han, D. R.; Shih, W. M.; Yan, H. Nat. Nanotechnol. 2011, 6, 763. (c) Tan, S. J.; Campolongo, M. J.; Luo, D.; Cheng, W. L. Nat. Nanotechnol. 2011, 6, 268.) DNA-directed self-assembly of oligonucleotide-functionalized gold NPs (AuNPs) introduced by Mirkin (Mirkin, C. A.; Letsinger, R. L.; Mucic, R. C.; Storhoff, J. J. Nature 1996, 382, 607.) and Alivisatos (Alivisatos, A. P.; Johnsson, K. P.; Peng, X. G.; Wilson, T. E.; Loweth, C. J.; Bruchez, M. P.; Schultz, P. G. Nature 1996, 382, 609). Since then, the process of attaching thiolated oligonucleotides to citrate stabilized AuNPs through successive salt aging has been well-developed. ((a) Hurst, S. J.; Lytton-Jean, A. K. R.; Mirkin, C. A. Anal. Chem. 2006, 78, 8313. (b) Cutler, J. I.; Auyeung, E.; Mirkin, C. A. J. Am. Chem. Soc. 2012, 134, 1376. (c) Elghanian, R.; Storhoff, J. J.; Mucic, R. C.; Letsinger, R. L.; Mirkin, C. A. Science 1997, 277, 1078.) These stable DNAAuNP conjugates have enabled the DNA-directed self-assembly of one-dimensional (1D) AuNP self-similar chains and arrays, ((a) Zhang, J. P.; Liu, Y.; Ke, Y. G.; Yan, H. Nano Lett. 2006, 6, 248. (b) Ding, B. Q.; Deng, Z. T.; Yan, H.; Cabrini, S.; Zuckermann, R. N.; Bokor, J. J. Am. Chem. Soc. 2010, 132, 3248.) two-dimensional (2D) AuNP superlattice sheets, (Cheng, W. L.; Campolongo, M. J.; Cha, J. J.; Tan, S. J.; Umbach, C. C.; Muller, D. A.; Luo, D. Nat. Mater. 2009, 8, 519.) three-dimensional (3D) AuNP tubes, (Sharma, J.; Chhabra, R.; Cheng, A.; Brownell, J.; Liu, Y.; Yan, H. Science 2009, 323, 112.) AuNP superlattice crystals,(Macfarlane, R. J.; Lee, B.; Jones, M. R.; Harris, N.; Schatz, G. C.; Mirkin, C. A. Science 2011, 334, 204.) and even chiral plasmonic AuNP nanostructures with tailored optical responses.(Kuzyk, A.; Schreiber, R.; Fan, Z. Y.; Pardatscher, G.; Roller, E. M.; Hogele, A.; Simmel, F. C.; Govorov, A. O.; Liedl, T. Nature 2012, 483, 311.)

However, less progress in organizing semiconductor NPs or quantum dots (QDs) into architectures with interesting fluorescence properties has been made. To facilitate DNA directed assembly of semiconductor QDs and achieve reliable architectures, the QDs should exhibit the following properties: (1) High chemical and photonic stability. The QDs should be highly resistant to chemical degradation and photobleaching during assembly of the underlying DNA nanoscaffolds, as the annealing process involves relatively high temperatures and ion concentrations; this requires the use of core/shell QDs. (2) Strong binding affinity between the DNA oligonucleotides and the QDs. The chemically modified oligonucleotides should not detach from the QD surface while in solution; thus, conventional thiolated oligonucleotide binding strategies are not adequate. (3) High colloidal stability over a wide range of buffer conditions. The chemically modified oligonucleotides should not precipitate or aggregate at high salt concentrations (>100mMNa⁺ or Mg²⁺) and should be stable at a variety of pHs. (4) High fluorescence quantum efficiency (>50%). This is important when the QDs are used as fluorescent markers for molecular detection or monitoring of biological processes at the single-particle level. (5) High spectral tunability to achieve a wide range of QD emissions. This is critical in applications such as biolabeling, light manipulation, and controlled energy transfer.

Recently, DNA-protein interactions have been used to arrange QDs on DNA tile arrays and origami. In 2008, we used biotinylated DNA tile arrays to direct the assembly of streptavidin-conjugated CdSe/ZnS core/shell QDs into well-defined periodic patterns. (Sharma, J.; Ke, Y. G.; Lin, C. X.; Chhabra, R.; Wang, Q. B.; Nangreave, J.; Liu, Y.; Yon, H. Angew. Chem., Int. Ed. 2008, 47, 5157.) In 2010, Bui et al. (Bui, H.; Onodera, C.; Kidwell, C.; Tan, Y.; Graugnard, E.; Kuang, W.; Lee, J.; Knowlton, W. B.; Yurke, B.; Hughes, W. L. Nano Lett. 2010, 10, 3367.) used biotinylated DNA origami nanotubes to assemble streptavidin-conjugated CdSe/ZnS QDs into arrays. In 2012, Ko et al. (Ko, S. H.; Gallatin, G. M.; Liddle, J. A. Adv. Funct. Mater. 2012, 22, 1015.) used biotinylated DNA origami to assemble streptavidin-functionalized QDs. Unfortunately, the complexity of structures that can be formed by this method is limited, as the biotinstreptavidin interaction is not information-bearing.

Alternatively, QD—DNA conjugates can be designed to bind directly to an underlying DNA nanostructure through sequence specific Watson-Crick base pairing, providing a significant increase in the level of structural complexity that can be achieved. Several conjugation strategies for attaching DNA oligonucleotides to the surface of QDs have been developed. Mirkin (Mitchell, G. P.; Mirkin, C. A.; Letsinger, R. L. J. Am. Chem. Soc. 1999, 121, 8122.) reported the attachment of thiol-modified (3′-propylthiol or 5′-hexylthiol) single-stranded DNA (ssDNA) to the surface of CdSe/ZnS QDs, similar to ssDNA—AuNP conjugates. We reported the attachment of thiol-modified ssDNA to the surface of CdSe/ZnS core/shell QDs, where the conjugation occurred during a one-step core/shell formation process. (Wang, Q. B.; Liu, Y.; Ke, Y. G.; Yan, H. Angew. Chem., Int. Ed. 2008, 47, 316.) Recently, Kelley (Tikhomirov, G.; Hoogland, S.; Lee, P. E.; Fischer, A.; Sargent, E. H.; Kelley, S. 0. Nat. Nanotechnol. 2011, 6, 485.) reported a synthetic route to produce phosphorothiolated phosphorodiester DNA (ps-po-DNA)-functionalized CdTe QDs, but these core-only QDs without shells had a low quantum yield (<50%). The above QD—DNA conjugates were less stable than their AuNPDNA counterparts under similar buffer conditions. This is the case because AuS bonds (ΔH=418 kJ/mol) are much stronger than Au—O bonds (ΔH=221.8 kJ/mol), enabling the thiolated DNA to displace the original citrate ligand on the AuNP surface and form stable Au—DNA conjugation, whereas the strengths of CdS (ΔH=208.4 kJ/ mol) and ZnS bonds (ΔH=205 kJ/mol) are similar to those of CdO (ΔH=235.6 kJ/mol) and ZnO bonds (ΔH=159 kJ/ mol), allowing these thiol ligands on the QD surface to be displaced by other ionic species present in the aqueous buffer.

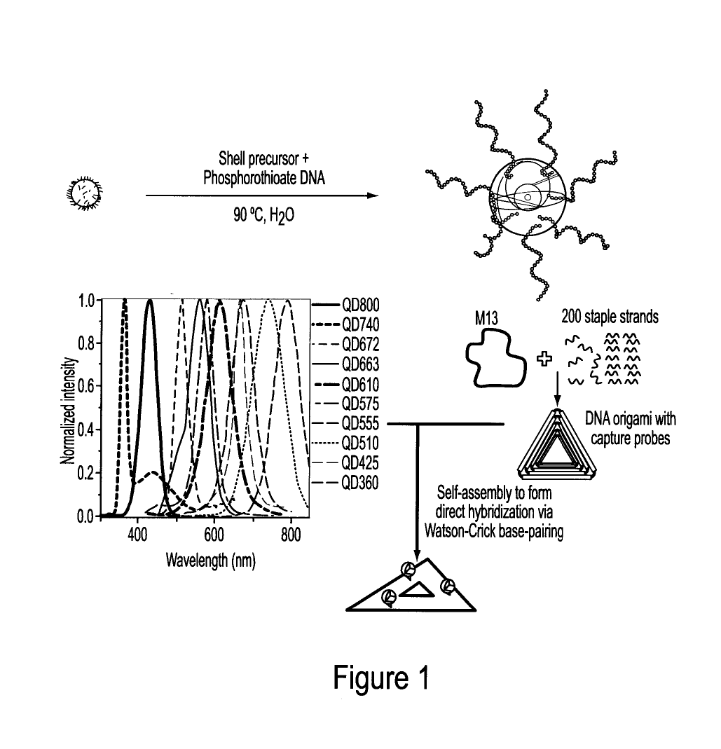

Herein we report a new strategy to achieve robust DNA functionalized core/shell QDs that satisfy all five requirements for DNA-directed self-assembly listed above. Our strategy for forming these QD—DNA conjugates (FIG. 1) takes advantage of chimeric ps-po-ssDNA strands that are directly inserted within a thick CdS or ZnS QD shell during its synthesis over the core. This synthetic route results in core/shell QD—DNA conjugates that are chemically, photonically, and colloidally stable as well as highly fluorescent [with photoluminescence (PL) quantum yields of up to 70%] for a wide range of semiconductor materials with tunable fluorescent emissions spanning from the UV to the NIR (360-800 nm). We also demonstrate the organization of these QD—DNA conjugates by complementary base pairing to triangular- and rectangular-shaped DNA origami structures. The synthesis proceeded as follows: first, water-soluble mercaptopropionic acid (MPA)-capped CdTe core QDs were encapsulated with thick CdS shells in the presence of ps-po-ssDNA [for details, see FIGS. S1-S9]. The magic-sized MPA-capped CdTe nanocrystals (1.6 nm diameter with peak PL at 480 nm) were synthesized as described previously. (Deng, Z. T.; Schulz, O.; Lin, S.; Ding, B. Q.; Liu, X. W.; Wei, X. X.; Ros, R.; Yan, H.; Liu, Y. J. Am. Chem. Soc. 2010, 132, 5592) In a typical reaction, an aliquot of CdTe core QDs was purified and redissolved in 100 μL of nanopure water. Prescribed amounts of Cd²⁺-MPA complex (the precursor of both Cd and S for CdS shell growth) and ps-po-ssDNA (the surface ligand) were added to the core mixture. The ps-po-ssDNA oligonucleotides (5′-T28-G5-ps-3′) contained a stretch of five consecutive Gresidues followed by five consecutive ps backbone modifications and 28 unmodified T residues linked by conventional phosphodiester bonds. The pH was adjusted to 12, and the mixture was heated at 90° C. for 70 min. During this time, the Cd²⁺ MPA complex slowly decomposed, and a CdS shell of particular thickness was formed around the CdTe core. The five S atoms in the ps domain of the DNA were “inserted” into the CdS shell during its formation, while most (if not all) of the poly-T domain extended away from the surface of the shell and was available for hybridization to the complementary DNA within the underlying DNA nanostructure. As we reported previously, (Deng, Z. T.; Schulz, O.; Lin, S.; Ding, B. Q.; Liu, X. W.; Wei, X. X.; Ros, R.; Yan, H.; Liu, Y. J. Am. Chem. Soc. 2010, 132, 5592.) at this relatively mild temperature the monolayer-by-monolayer formation of the CdS shell is fully controlled by the slow decomposition of the Cd²⁺ MPA complex, and the shell thickness is determined by the total reaction time. We observed that seven CdS shell monolayers formed in 70 min, so the estimated synthesis time was 10 min/monolayer. In view of the relatively low rate of growth, the S atoms in the ps domains of the oligonucleotides had ample opportunity to bond to the Cd atoms and were readily incorporated into the CdS shell. From the UV-vis absorption spectra, the number of ssDNA on one QD was estimated to be 9 (see FIG. S9).

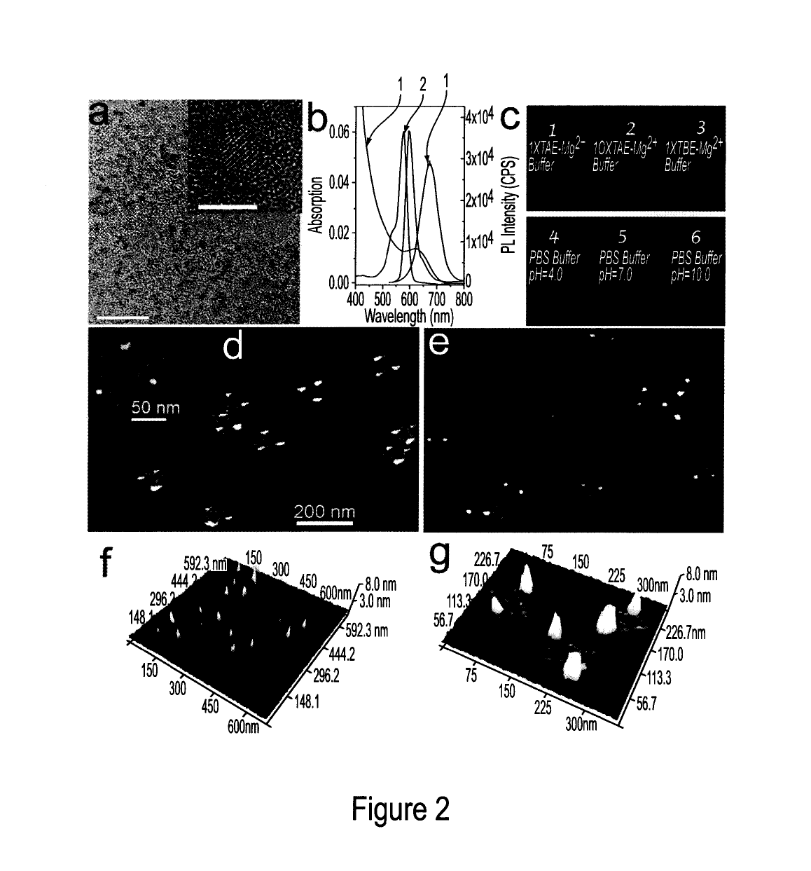

The resulting core/shell CdTe/CdS QD—DNA conjugates had an estimated diameter of 6.5 nm, corresponding to a seven monolayer CdS (7CdS) shell, with a band-edge emission maximum at 672 nm and a PL quantum yield of 70%. The observed ˜200 nm red shift of the emission peak is assigned to quasi-type-II QDs. ((a) Deng, Z. T.; Lie, F. L.; Shen, S. Y.; Ghosh, I.; Mansuripur, M.; Muscat, A. J. Langmuir 2009, 25, 434. (b) Deng, Z. T.; Yan, H.; Liu, Y. J. Am. Chem. Soc. 2009, 131, 17744.) Transmission electron microscopy (TEM) and high-resolution TEM (HRTEM) images revealed that the QD—DNA conjugates were monodispersed, single-crystalline particles (FIG. 2a). The purified thick-shell QD—DNA conjugates were found to be stable under a variety of buffer conditions, including lx PBS buffer at pH 4.0, 7.0, and 10.0; 1×TAEMg2+ buffer; 1×TBEMg2+ buffer; and 10×TAEMg2+ buffer (125 mM Mg2+) (FIG. 2c). Thus, our QD—DNA conjugates are colloidally stable over a wide pH range (4-12) at high salt concentrations (>100 mM Na+ or Mg2+).

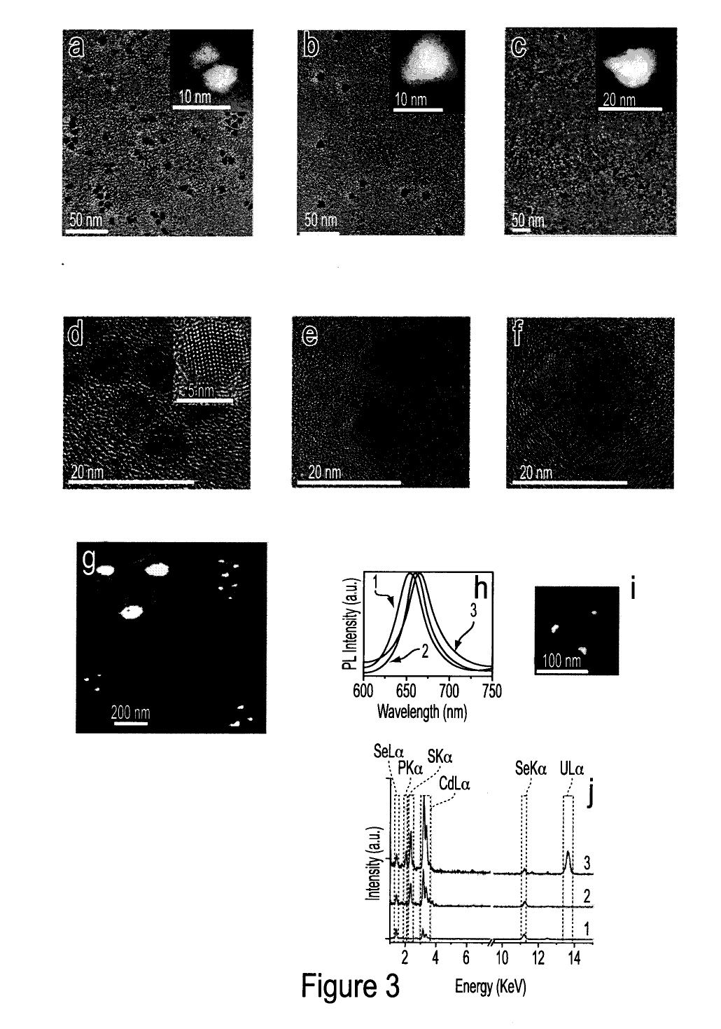

These CdTe/7CdS QD-DNA conjugates were subsequently assembled at precise positions on DNA origami structures via hybridization to complementary poly-A capture probes extending from the origami surface (three capture probes per QD—DNA). We demonstrated the organization of three or two QD—DNA conjugates on triangular or rectangular DNA origami, respectively (FIG. 2d-g). Atomic force microscopy (AFM) and scanning TEM (STEM) confirmed that >95% of the triangular DNA origami structures displayed three QDs, one on each arm (FIG. 2d,f), and that 90% of the rectangular origami structures displayed two QD—DNA conjugates, one each at opposite corners (FIG. 2e,g), as prescribed by the design (for design details and additional images, see SI pages S29-S45 and FIGS. S24-S26). The AFM height profiles showed that the QD—DNA conjugates had a narrow size range (6-7 nm). We also synthesized thick-shell CdSe/CdS QD—DNA conjugates containing 20-monolayer CdS (20 CdS) shells. As reported by Hollingsworth (Chen, Y.; Vela, J.; Htoon, H.; Casson, J. L.; Werder, D. J.; Bussian, D. A.; Klimov, V. I.; Hollingsworth, J. A. J. Am. Chem. Soc. 2008, 130, 5026.) and Dubertret, (Mahler, B.; Spinicelli, P.; Buil, S.; Quelin, X.; Hermier, J. P.; Dubertret, B. Nat. Mater. 2008, 7, 659.) “giant”-shell or thick-shell QDs are more chemically stable and exhibit reduced blinking behavior at the single-particle level. Thick-shell QDs have been obtained by the successive ionic layer adsorption and reaction (SILAR) method, ((a) Chen, Y.; Vela, J.; Htoon, H.; Casson, J. L.; Werder, D. J.; Bussian, D. A.; Klimov, V. I.; Hollingsworth, J. A. J. Am. Chem. Soc. 2008, 130, 5026. (b) Mahler, B.; Spinicelli, P.; Buil, S.; Quelin, X.; Hermier, J. P.; Dubertret, B. Nat. Mater. 2008, 7, 659. (c) Deng, Z.; Cao, L.; Tang, F.; Zou, B. J. Phys. Chem. B 2005, 109, 16671. (d) Smith, A. M.; Mohs, A. M.; Nie, S. Nat. Nanotechnol. 2009, 4, 56.) which requires high temperatures (240° C.) and a growth process in an organic solvent. Here we developed a new method to achieve thick-shell CdSe/20 CdS QDs at lower temperatures (90° C.) in aqueous solution. More significantly, we incorporated the ssDNA within the shell itself during the encapsulation process. First, oleic acid (OLA)-capped CdSe core QDs (6 nm diameter) were synthesized in paraffin liquid at 320° C. (FIG. 3a,d; for details, see SI pages S13-S21 and FIGS. S10-S17). The OLA-capped CdSe cores had diameters of 6.0 nm and fluorescence emission at 650 nm. Next, nine-monolayer CdS shells were deposited on the CdSe cores in aqueous solution at 90° C. with MPA as the capping ligand. After the ligand exchange and CdS shell growth, the tetrahedral-shaped MPA-capped QDs exhibited emission at 660 nm. The approximate length of these tetrahedral-shaped QDs was 12 nm (FIG. 3b,e). Finally, additional shells were incorporated on the QDs in the presence of ps-po-ssDNA. The resulting DNA oligonucleotide-functionalized thick-shell CdSe/20 CdS QDs emitted at 663 nm. The length of the QD—DNA conjugates increased to 18 nm (FIG. 3c,f). The relatively small size of the PL red shift upon shell growth was due to the large core size of these core/shell QDs (6 nm diameter), as small core (3 nm diameter) CdSe QDs encapsulated with a thick shell showed a PL red shift of 70 nm (from 565 to 635 nm; FIG. S10). This may be a strain-induced PL shift.

We demonstrated that these robust, thick-shell QD—DNA conjugates are readily organized by addressable DNA origami structures to form discrete, well-ordered nanoarchitectures. In addition, DNA origami are an ideal platform to confirm the successful DNA functionalization of the QDs, which is more straightforward and reliable than the previous QD—DNA/dye FRET method.8b As demonstrated in FIG. 3j, energy dispersive X-ray spectroscopy (EDS) spectra of the self assembled origami nanostructures revealed the presence of Cd, Se, S, and P from the CdSe/20 CdS QD—DNA conjugates.

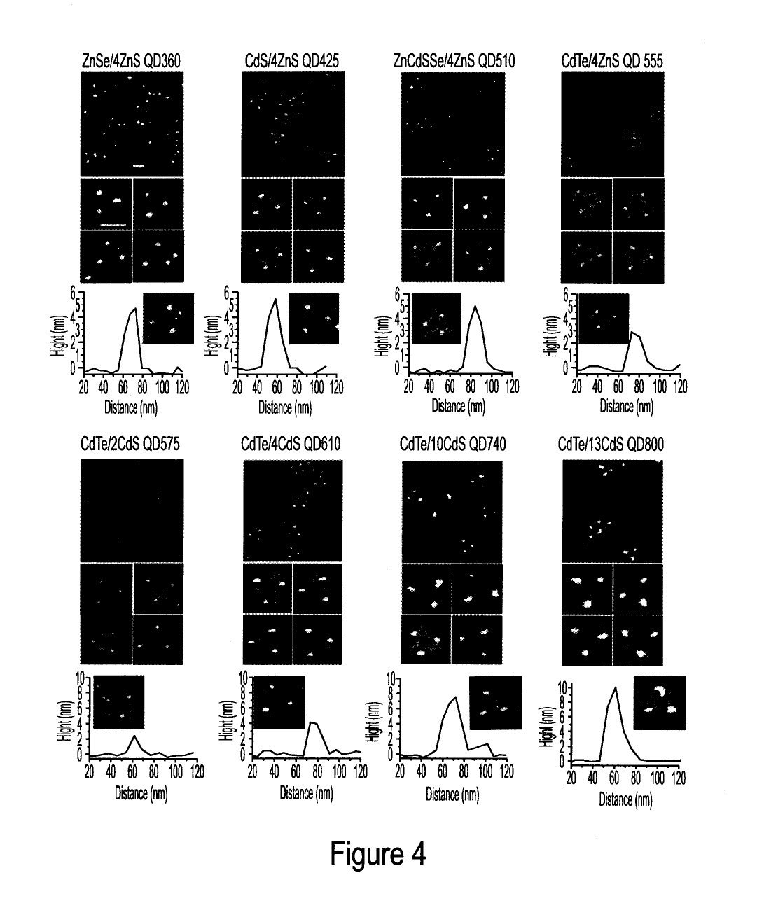

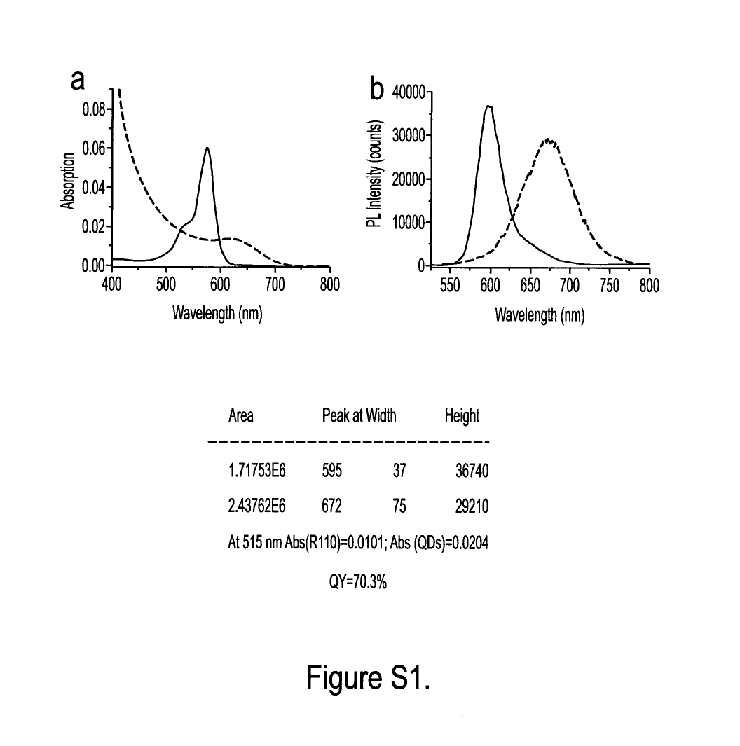

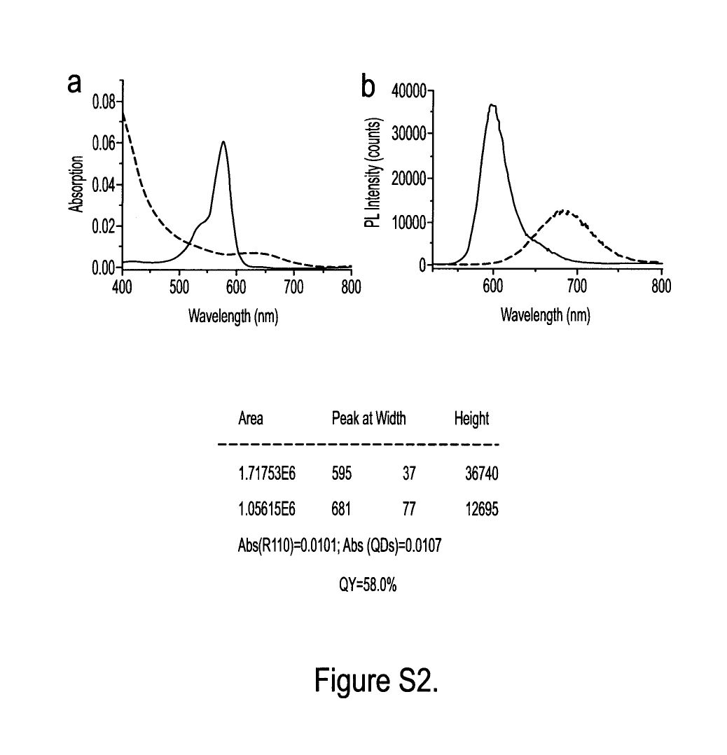

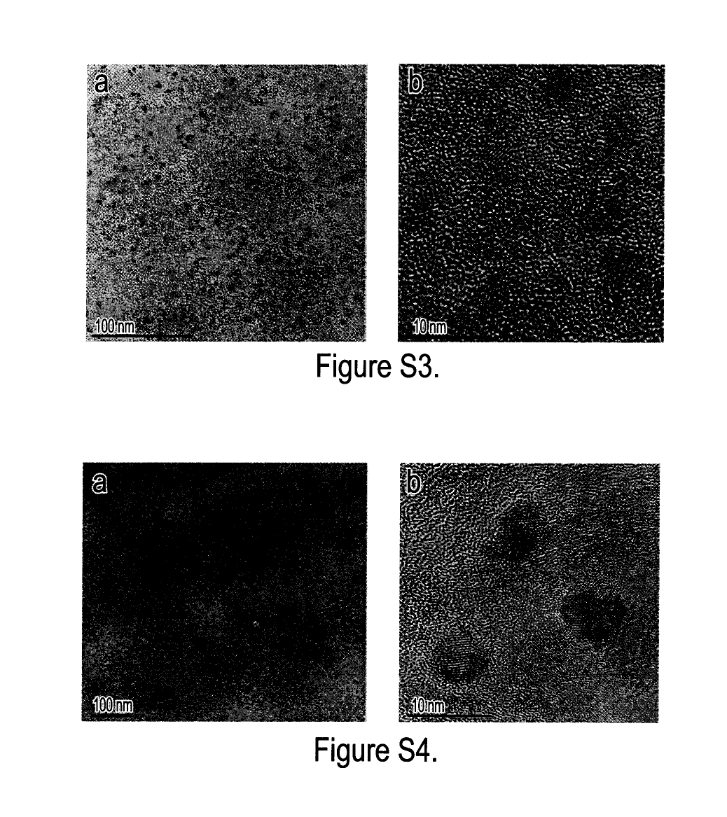

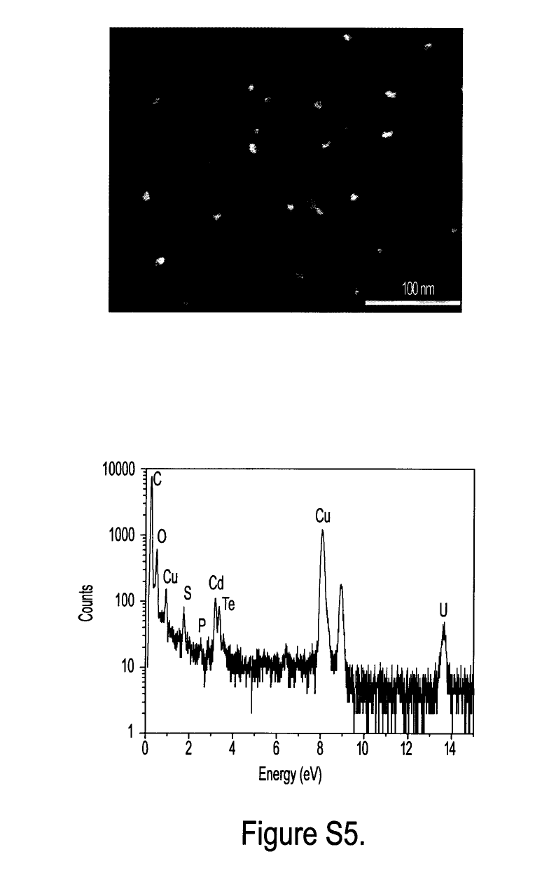

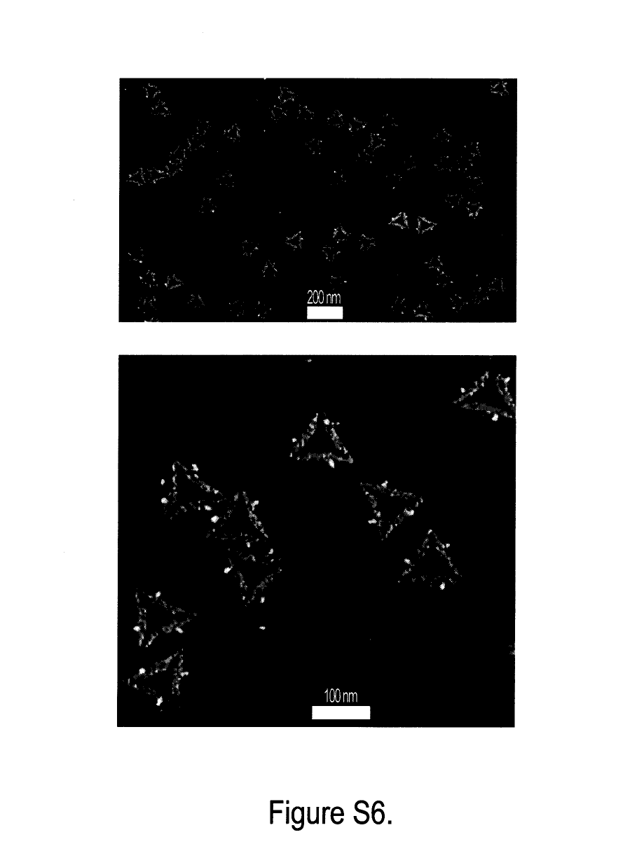

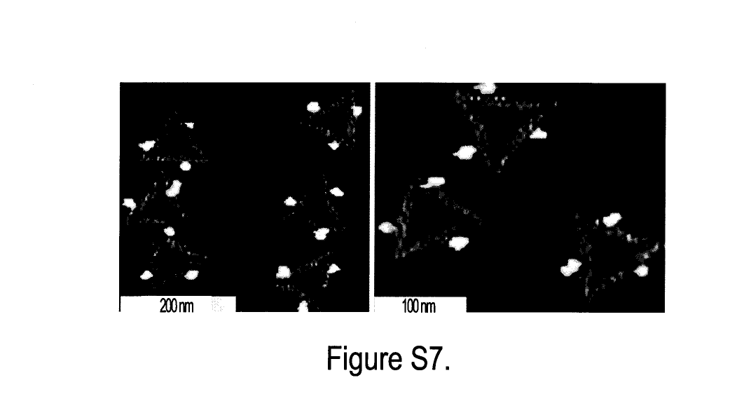

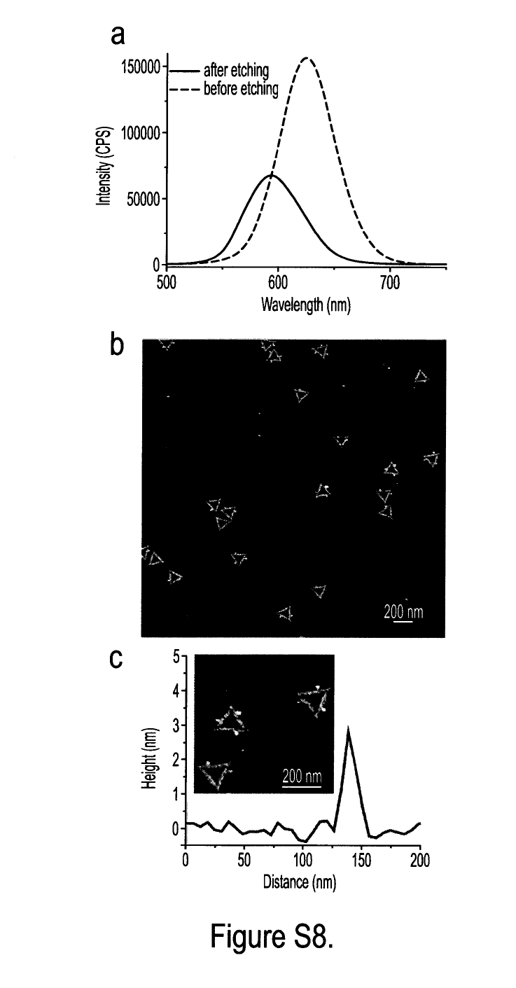

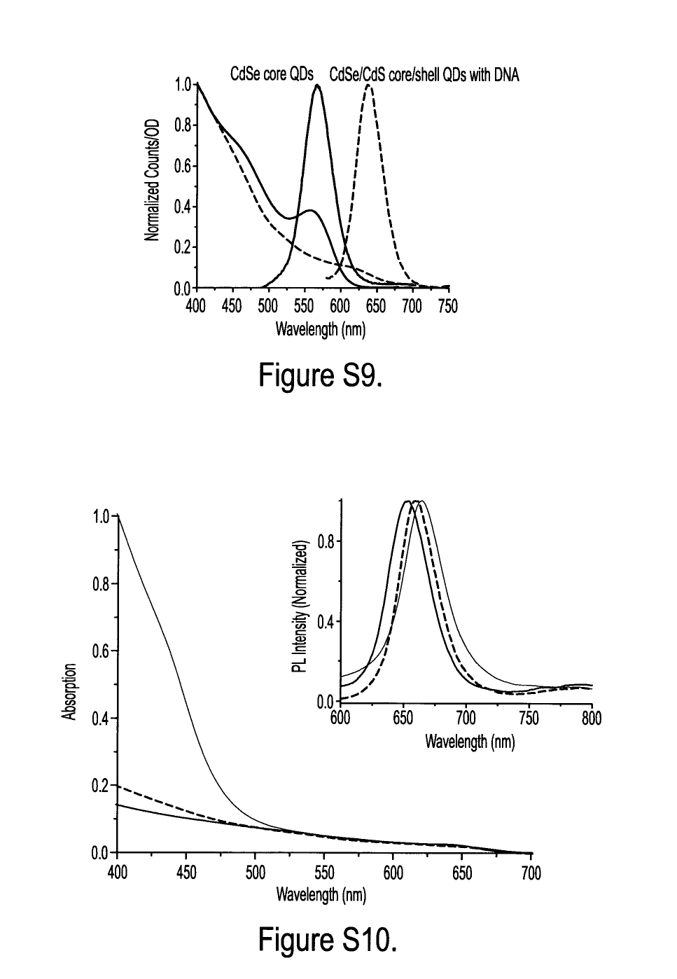

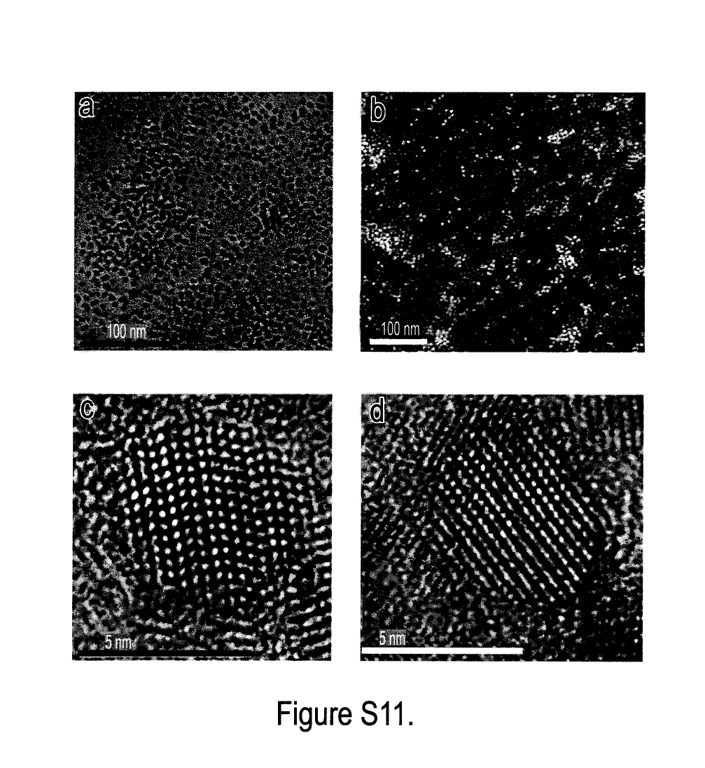

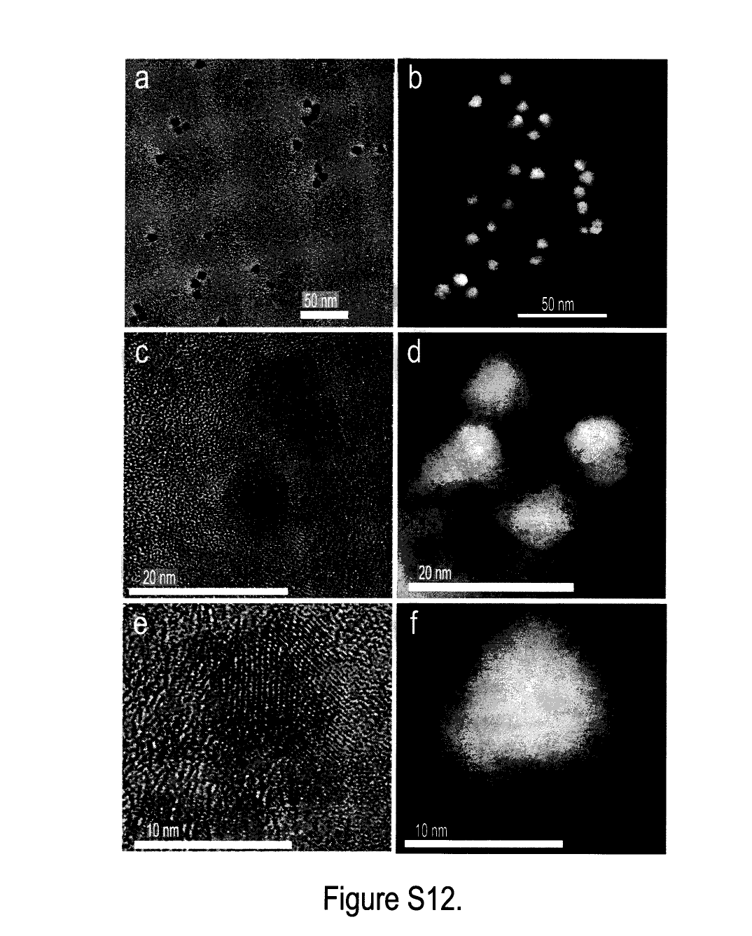

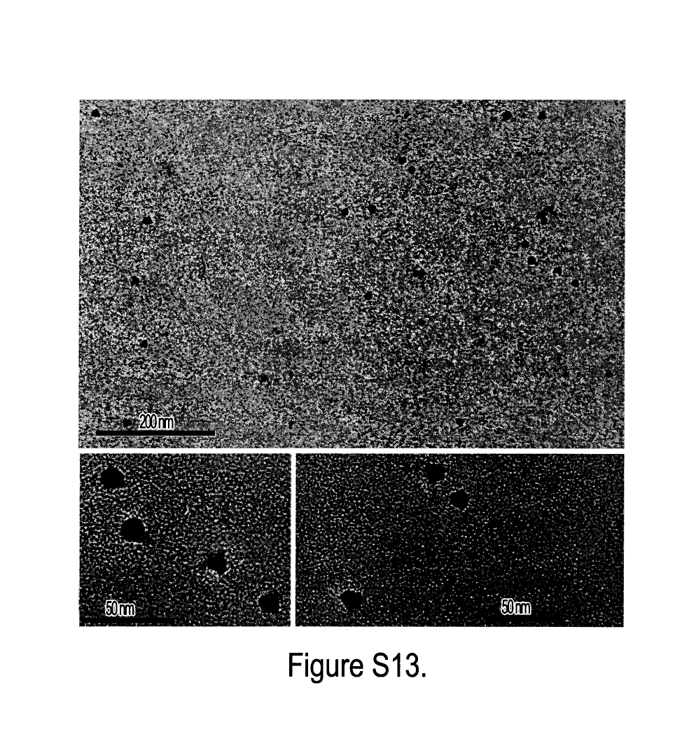

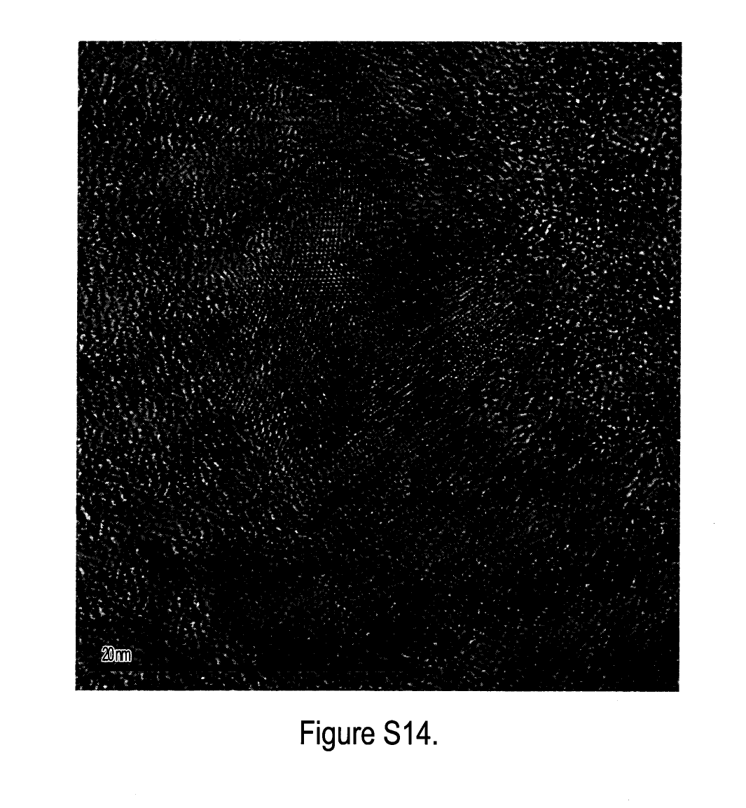

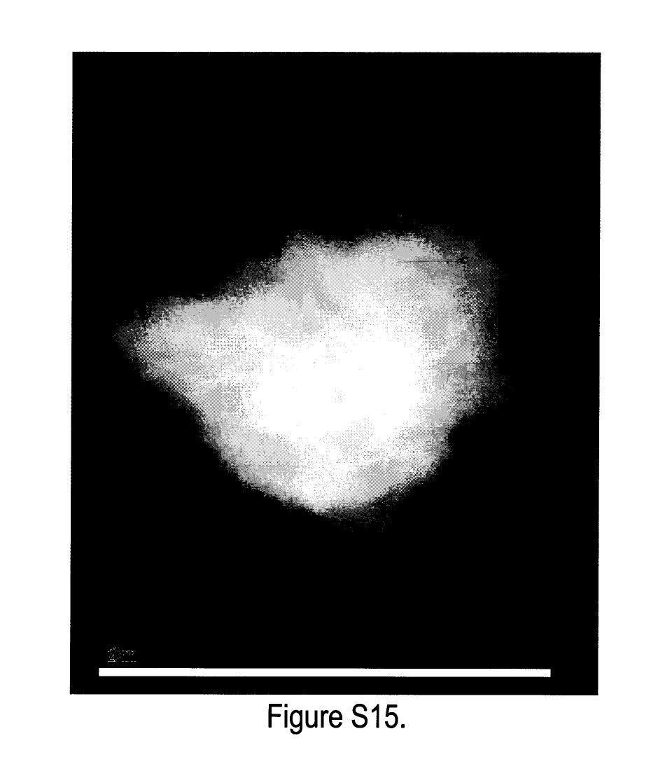

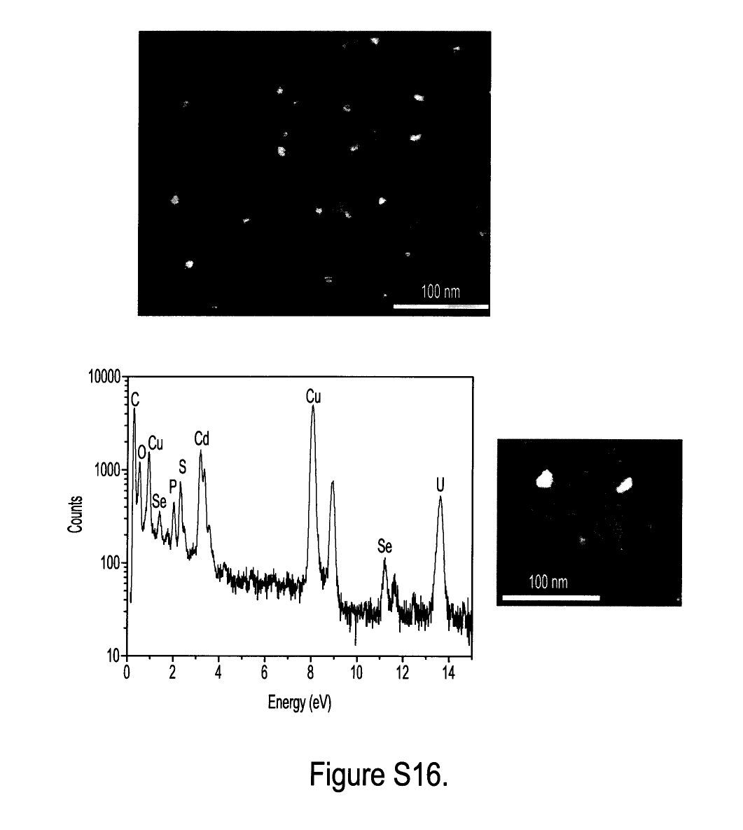

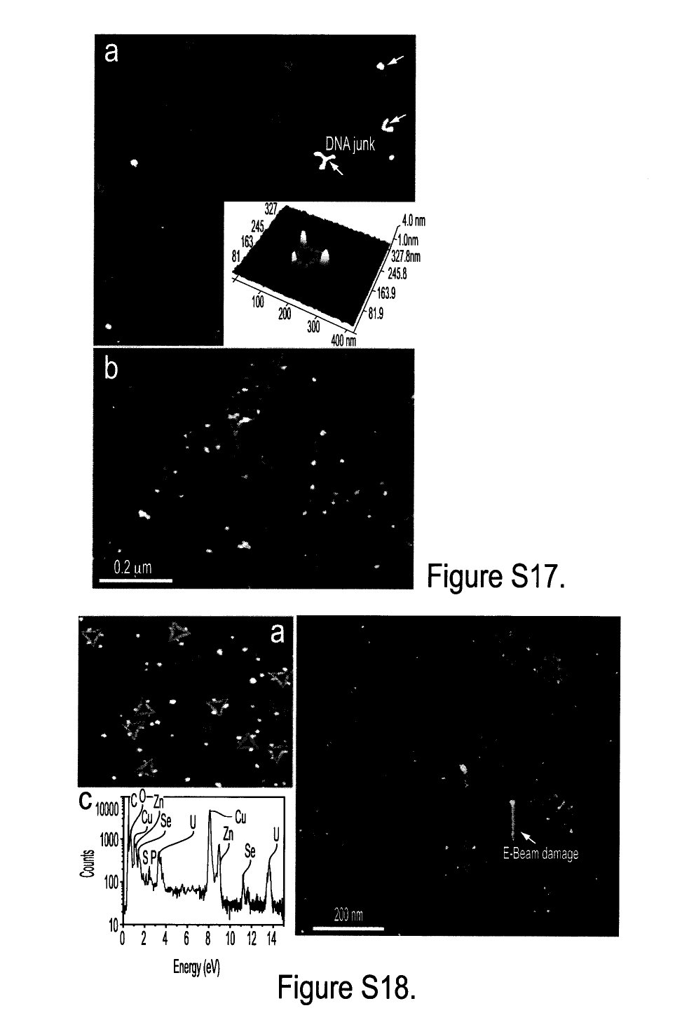

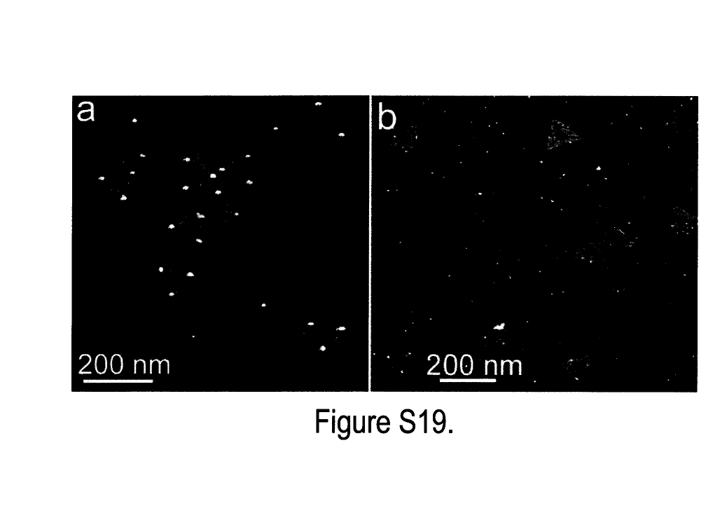

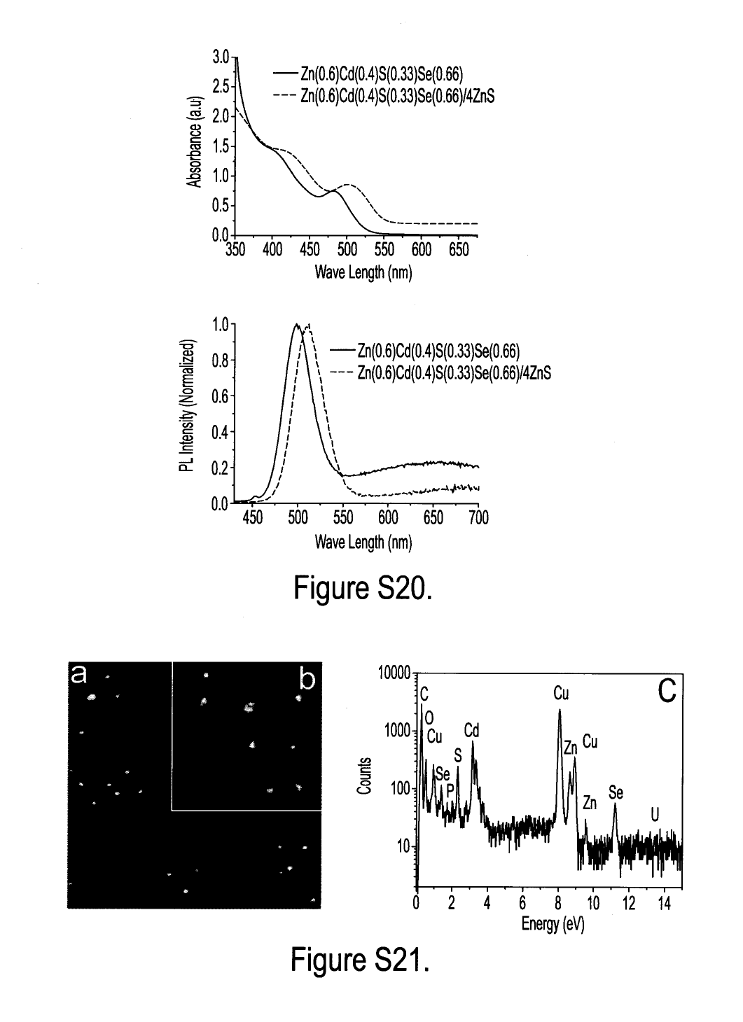

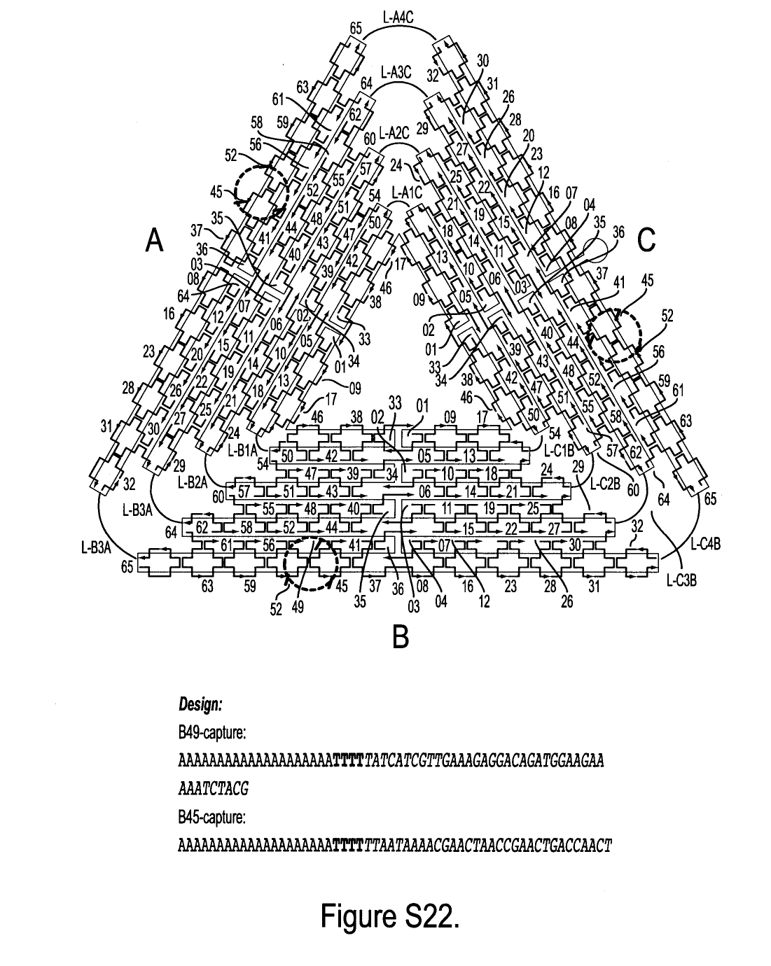

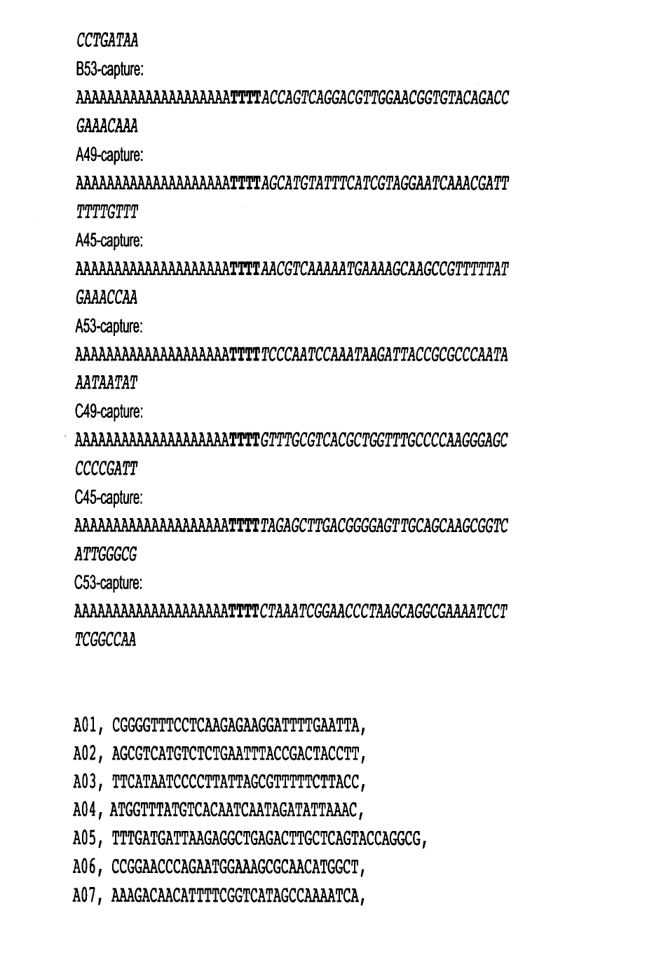

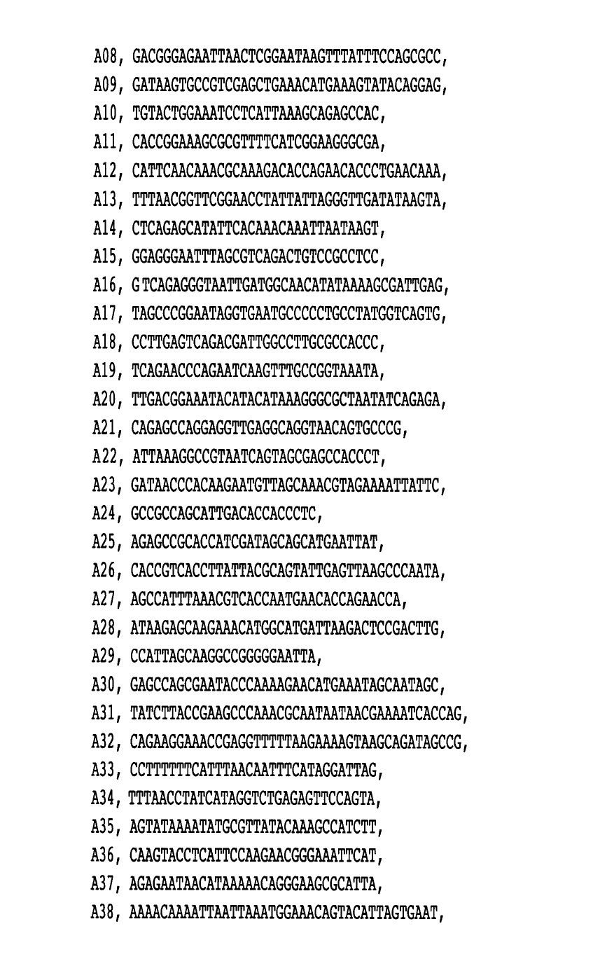

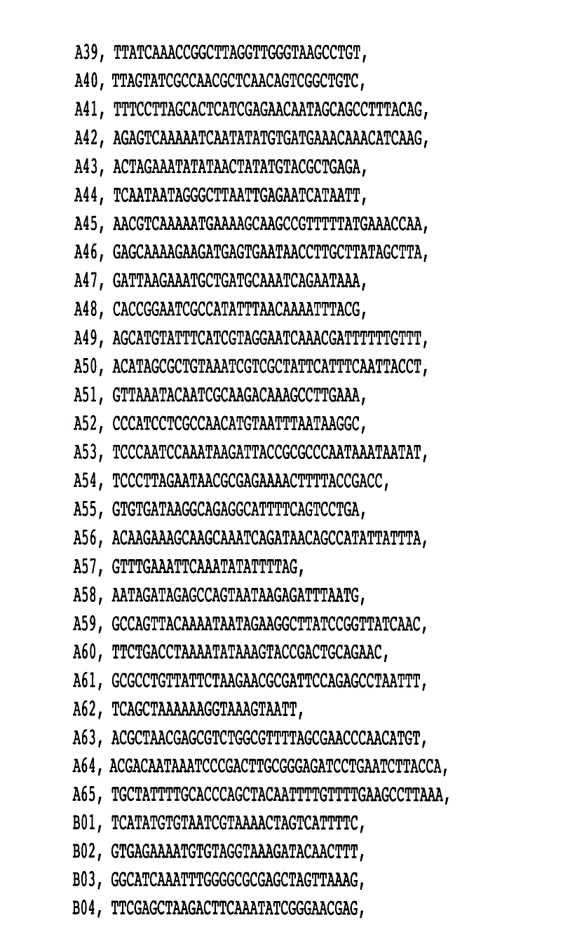

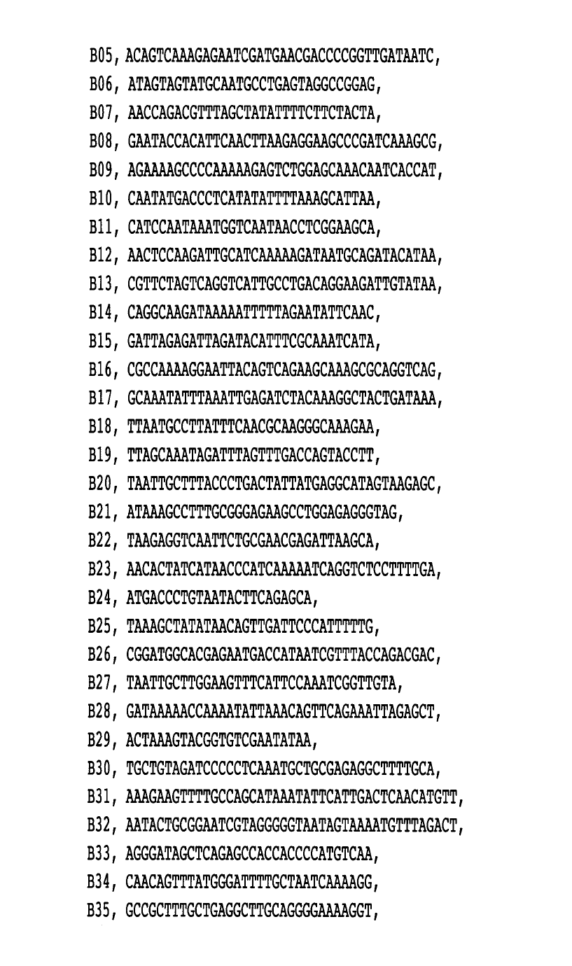

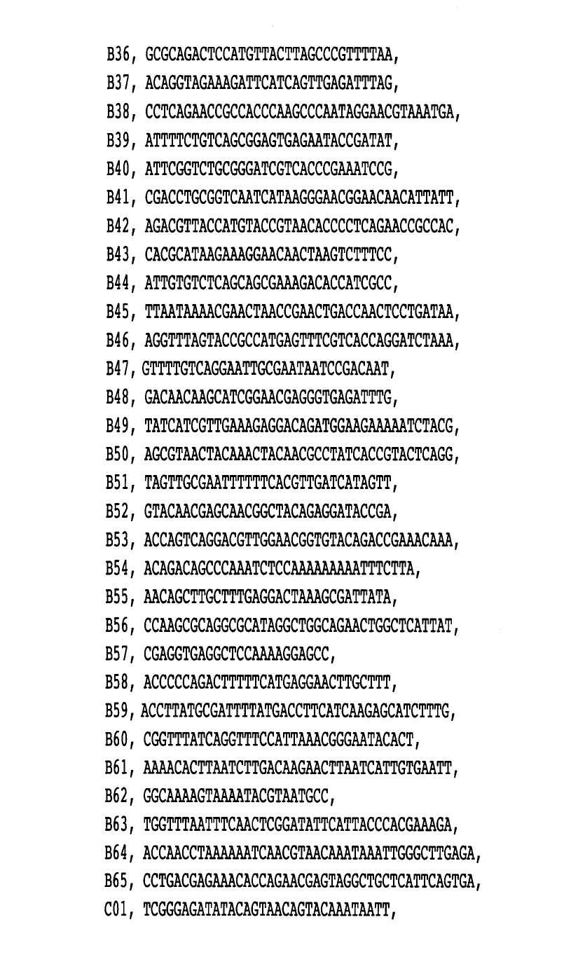

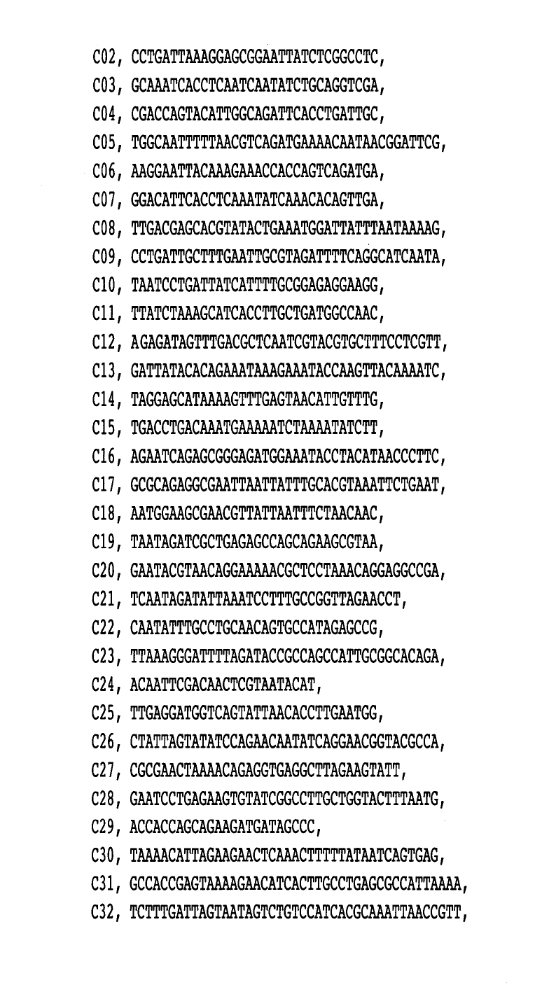

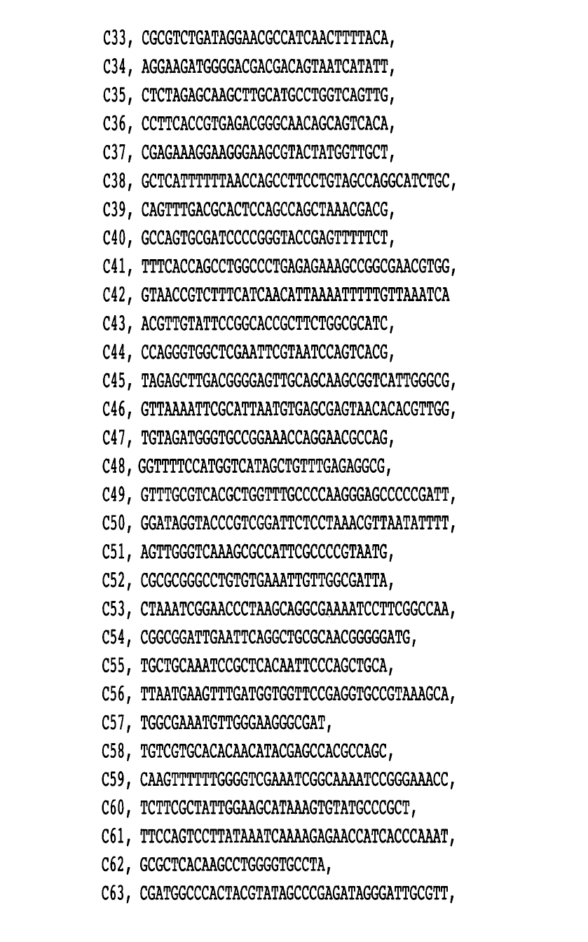

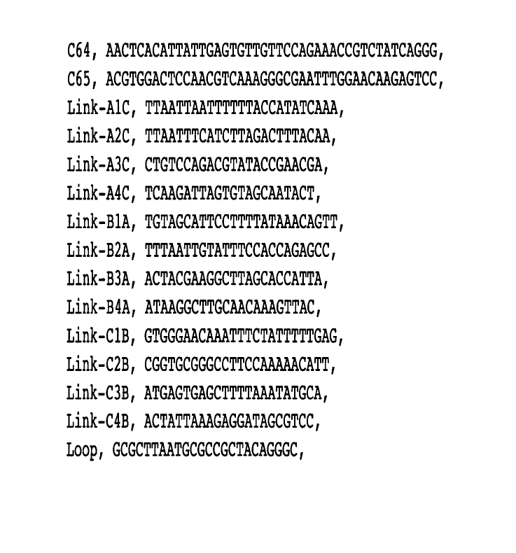

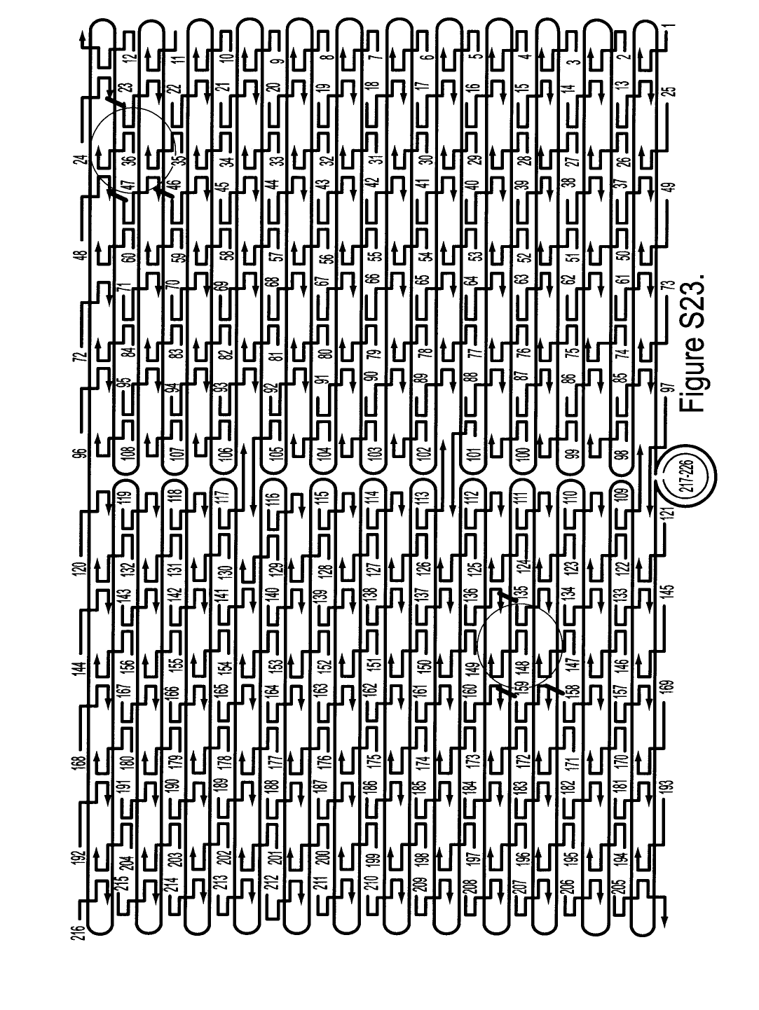

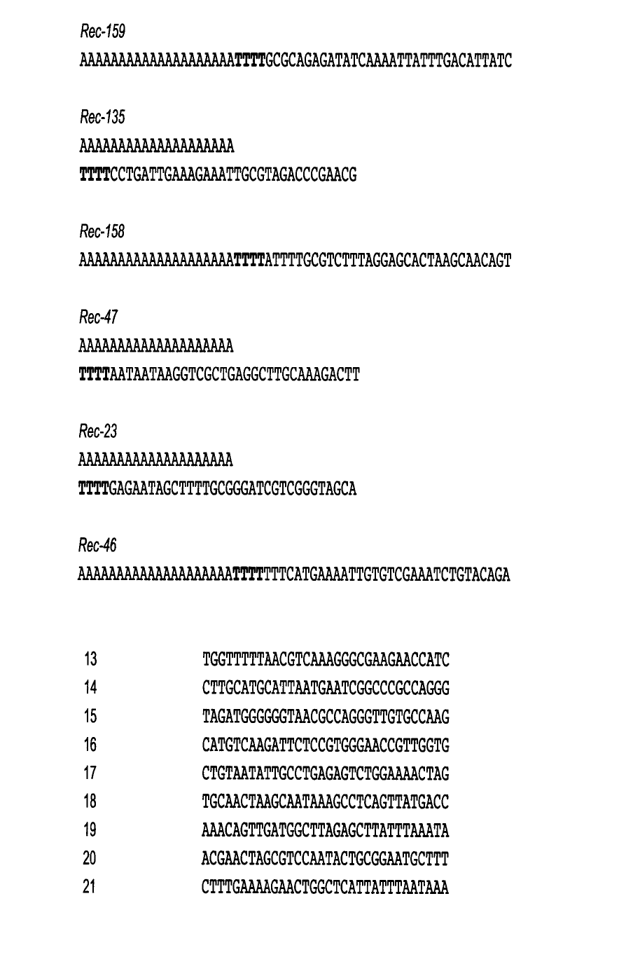

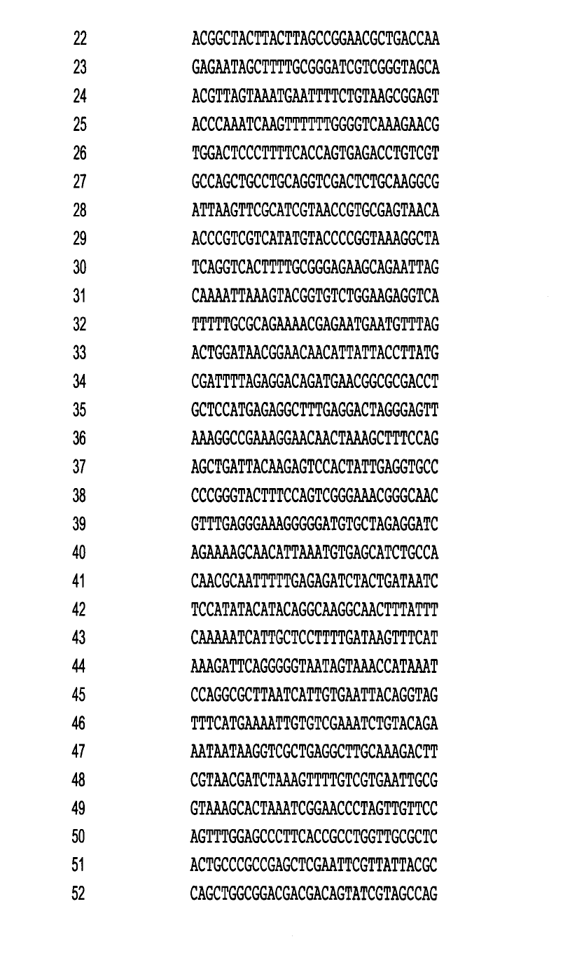

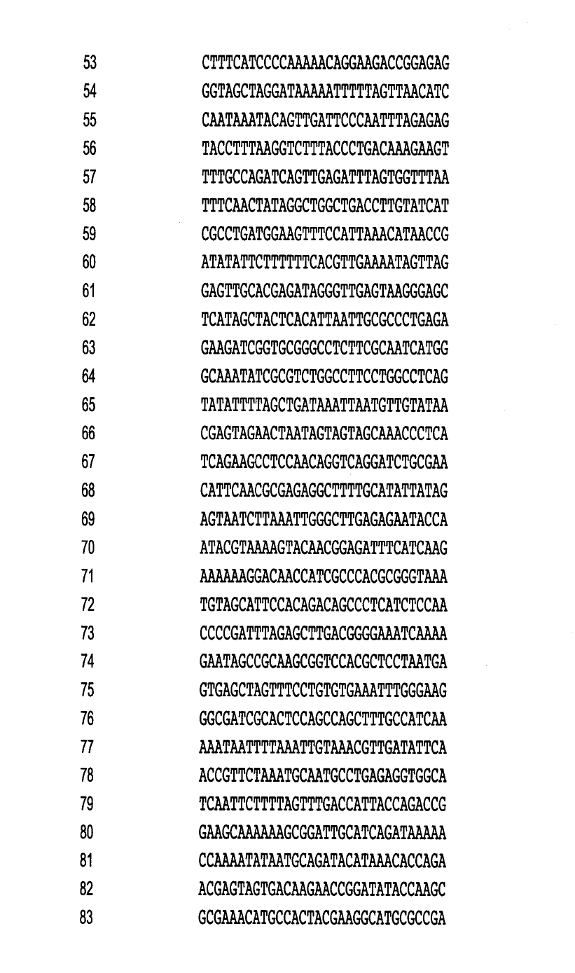

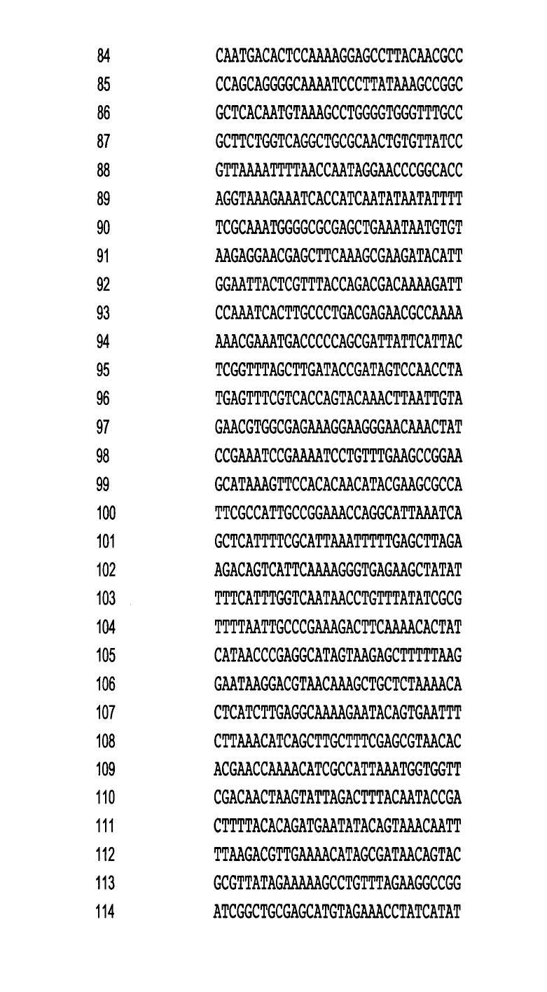

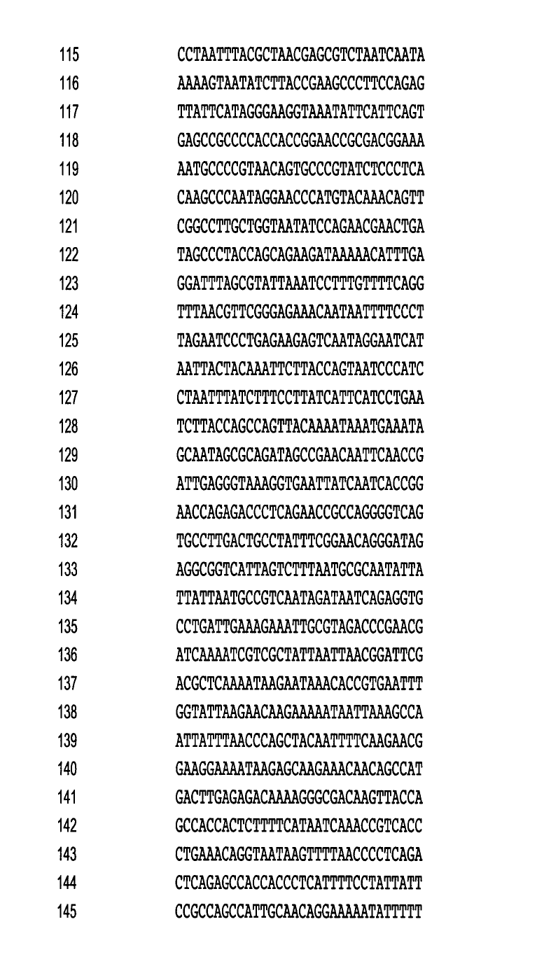

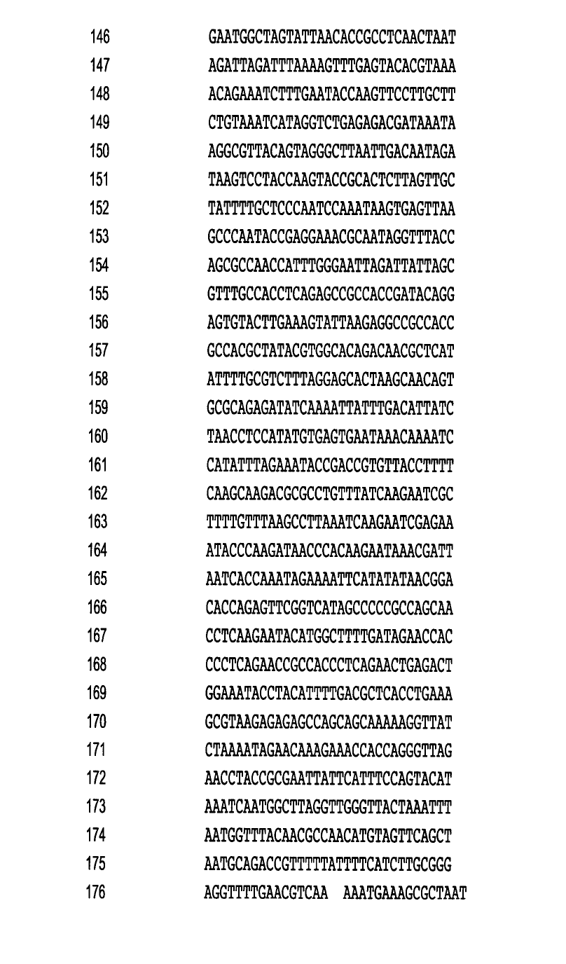

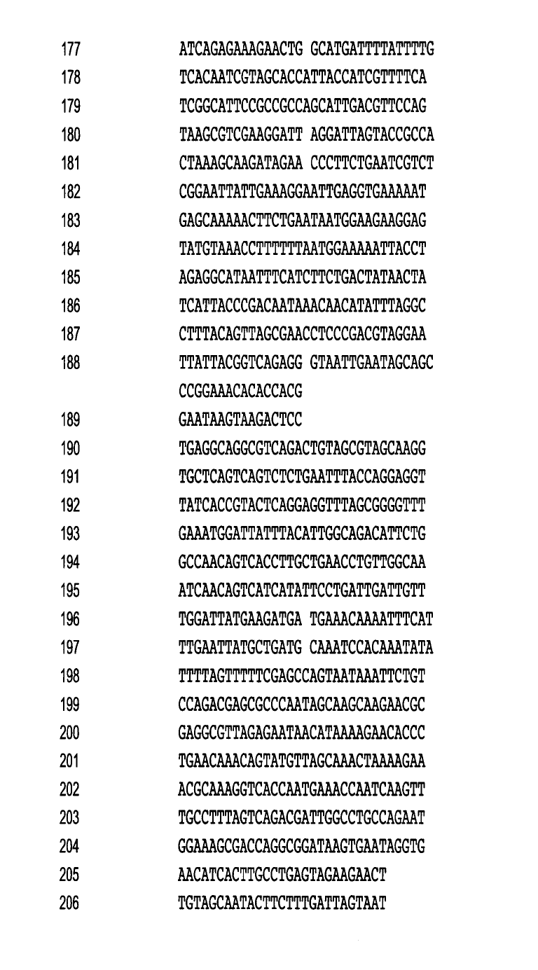

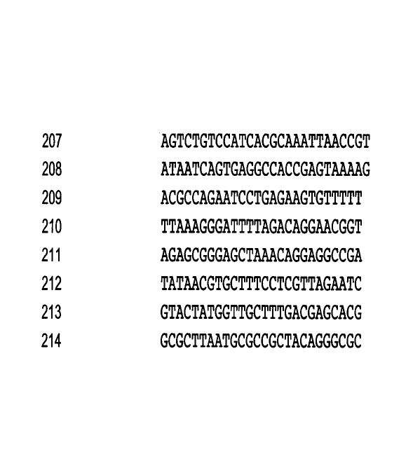

This strategy is quite versatile and can be applied to QDs composed of other semiconductor materials (FIG. 4). For example, we found that ZnS shells could be deposited on a variety of different core materials using the same strategy. Using a water-soluble ZnSe core, we produced ZnSe/4ZnS QD—DNA conjugates displaying UV emission at 360 nm (FIG. 4; for details, see SI pages S22S28 and FIGS. S18-S23). Using OLA-capped CdS or quaternary-alloy ZnCdSSe QD cores,12b we synthesized CdS/4ZnS QD—DNA conjugates with blue emission at 425 nm and ZnCdSSe/4ZnS QD—DNA conjugates with green emission at 510 nm, respectively. Furthermore, we produced CdTe/4ZnS QD—DNA conjugates with yellow emission at 555 nm. Finally, we used magic-sized CdTe core QDs to synthesize a series of DNA-functionalized QDs, including CdTe/2CdS, CdTe/4CdS, and thick-shell CdTe/10CdS and CdTe/13CdS QDs with emission maxima in the orange, red, and NIR at 575, 610, 740, and 800 nm, respectively. The organization of each of these QD-DNA conjugates by DNA origami is shown in FIG. 4. The AFM height profiles of the particles corresponded well to the sizes measured using TEM. Since CdS and ZnS are wide-band-gap semiconductor materials that are generally used in QD shells, it is reasonable to expect that many other core/shell QDs with various core compositions can be synthesized, such as binary PbS, InP, and InAs QDs for IR emission; doped ZnSe:Mn QDs; ternary-alloy CuInSe and ZnCdSe QDs; quaternary-alloy CuInSSe QDs; and so on. All of these should be compatible with the oligonucleotide functionalization strategy reported here.

In summary, we have developed a simple and efficient method to synthesize robust core/shell QD—DNA conjugates that can withstand the conditions necessary for DNA-directed assembly. In contrast to QD functionalization strategies in which the DNA ligands are simply attached to the QD surface, our method embeds the DNA within the shell itself, providing greater stabilization. Our strategy can be used with a wide variety of semiconductor materials displaying PL emission ranging from the UV to the NIR. Also, discrete numbers of QD—DNA conjugates can be organized by DNA origami nanostructures, an essential component of hierarchical NP assembly efforts. This work will facilitate the construction of discrete, multicomponent semiconductor or semiconductormetal hybrid nanostructures for energy, nanophotonics, and biosensing applications.

The invention will now be illustrated by the following non-limiting Example.

### EXAMPLE 1

Robust DNA Functionalized Quantum Dots Compatible with DNA Directed Self-Assembly

The assembly and isolation of DNA oligonucleotide functionalized gold nanoparticles (AuNPs) has become a well-developed technology due to the strong bonding interactions between gold and thiolated DNA. However, achieving DNA functionalized semiconductor quantum dots (QDs) that are robust enough to withstand precipitation at high temperature and ionic strength through simple ‘attachment’ of modified DNA on the QD surface remains a challenge. Described herein is a method that facilitates the synthesis of stable core/shell (1 to 20 monolayers) QD-DNA conjugates by ‘embedding’ the end part (5-10 nucleotides) of the phosphorothiolated oligonucleotides within the outer shell of the QDs. These reliable QD-DNA conjugates exhibit excellent chemical and photonic stability, colloidal stability over a wide pH range (4-12) and high salt (>100 mM Na⁺ or Mg²⁺) conditions, bright florescence emission with quantum yield up to 70%, and broad spectra tunability with emission ranging from ultraviolet to near infrared.

Organizing inorganic nanoparticles (NPs) with nanoscale precision is of great interest to energy, nanophotonics and nanobiotechnology applications (Service, R. F., Science 309, 95-95 (2005); Srivastava, S. et al., Science 327, 1355-1359 (2010); Liu, Y., Nature Nanotechnology 6, 463-464 (2011)). One of the most promising approaches for the fully programmable self-assembly of NPs, DNA nanotechnology, relies on Watson-Crick base-pairing interactions between DNA functionalized NPs and underlying DNA nano-scaffolds (Seeman, N. C., Nature 421, 427-431 (2003); Pinheiro, et al., Nature Nanotechnol. 6, 763-772 (2011); Tan, et al., Nature Nanotechnol. 6, 268-276 (2011). DNA-directed self-assembly of oligonucleotide functionalized gold nanoparticles (AuNPs) was first introduced by Mirkin et al. and Alivisatos et al. in 1996 (Mirkin, et al., Nature 382, 607-609 (1996); Alivisatos, A. P. et al. Nature 382, 609-611 (1996)). Since then, the process of attaching thiolated oligonucleotides on citrate-stabilized AuNPs through successive salt-aging has been well-developed (Hurst, S. J., et al., Anal. Chem. 78, 8313-8318 (2006); Cutler, et al., J. Am. Chem. Soc. 134, 1376-1391 (2012); Elghanian, et al., Science 277, 1078-1081 (1997)). These stable DNA-AuNP conjugates have made possible the DNA directed self-assembly of one-dimensional (1D) AuNP self-similar chain and arrays (Zhang, et al., Nano Lett. 6, 248-251 (2006); Ding, B. Q. et al. J. Am. Chem. Soc. 132, 3248-3249 (2010)), two-dimensional (2D) AuNP superlattice sheets (Cheng, W. L. et al. Nature Mater. 8, 519-525 (2009)), three-dimensional (3D) AuNP tubes (Sharma, J. et al., Science 323, 112-116 (2009)), AuNP superlattice crystals (Macfarlane, R. J. et al. Science 334, 204-208 (2011)), and even chiral plasmonic AuNP nanostructures with tailored optical responses (Kuzyk, A. et al., Nature 483, 311-314 (2012)). However, progress in organizing semiconductor nanoparticles or quantum dots (QDs) into architectures with interesting fluorescence properties has fallen behind that of metallic NPs. To facilitate DNA-directed assembly of semiconductor QDs and achieve reliable architectures, the QDs should exhibit the following properties:

1. High chemical and photonic stability—the QDs should be highly resistant to chemical degradation and to photo-bleaching during assembly of the underlying DNA nanoscaffold, as the annealing process involves relatively high temperatures and ionic conditions. This property requires the use of core/shell QDs.

2. Strong binding affinity between the DNA oligonucleotides and the QDs—the chemically modified oligonucleotides should not detach from the QD surface while in solution. As such, conventional strategies such as thiolated oligonucleotide binding and carbodiimide (EDC) coupling are not adequate.

3. High colloidal stability over a wide range of buffer conditions—the chemically modified oligonucleotides should not precipitate or aggregate in high salt conditions (>100 mM Na⁺ or Mg²⁺), and also should be stable in a variety of pHs.

4. High fluorescence quantum efficiency (>50%) this is important for applications in which the QDs are used as fluorescent markers for molecular detection or monitoring biological processes at the single particle level.

5. High spectral tunability to achieve a wide range of QD emissions (UV-Vis-NIR)—this is critical to various applications including biolabelling, light manipulation and controlled energy transfer.

Recently, DNA-protein interactions have been used to arrange QDs on DNA tile arrays and origami. In 2008, biotinylated DNA-tile arrays to direct the assembly of commercially available streptavidin-conjugated CdSe/ZnS core/shell QDs into well-defined periodic patterns were used (Sharma, J. et al. Angew. Chem. Int. Ed. 47, 5157-5159 (2008)). In 2010, Bui et al. used biotinylated DNA origami nanotubes to assemble streptavidin-conjugated CdSe/ZnS QDs into arrays (Nano Lett. 10, 3367-3372 (2010)). Even more recently, Ko et al. used biotinylated DNA origami structures to direct the assembly of streptavidin-functionalized quantum dots (Adv. Funct. Mater. 22, 1015-1023 (2012)). Unfortunately, the complexity of structures that can be formed by this method is limited, as the biotin-streptavidin interaction is not an information bearing interface.

Alternatively, QD-DNA conjugates can be designed to bind directly to an underlying DNA nanostructure through sequence specific Watson-Crick base-pairing, making it is possible to significantly increase the level of structural complexity that can be achieved. Several conjugation strategies have been developed to attach DNA oligonucleotides to the surface of QDs. Mirkin et al, reported the attachment of thiol modified (3′ propylthiol or 5′ hexylthiol) single stranded DNA (ssDNA) to the surface of CdSe/ZnS QDs, similar to ssDNA-AuNP conjugates (Mitchell, et al., J Am. Chem. Soc. 121, 8122-8123 (1999)). Recently, the attachment of thiol modified ssDNA to the surface of CdSe/ZnS core/shell QDs, where the conjugation occurred during a one-step core/shell formation process, was reported (Wang, et al., Angew. Chem. Int. Ed. 47, 316-319 (2008)). However, these Cd or Zn based QD—DNA conjugates are not as stable as their AuNP-DNA counterparts in similar buffer conditions. This is because the Au—S (ΔH=418 KJ/mole) bonds are much stronger that the Au—O (ΔH=221.8 KJ/mole), so the thiolated DNA can kick out the original citrate ligand on the AuNPs surface to form stable Au-DNA conjugation. But the Cd-S (ΔH=208.4 KJ/mole) and Zn—S (ΔH=205 KJ/mole) bonds are similar to Cd—O (ΔH=235.6 KJ/mole) and Zn—O (ΔH=159 KJ/mole) energies. As a result, these thiol modified ligands on the QD surface are readily displaced by other ionic species present in the aqueous buffer.

Another strategy to generate QD—DNA conjugates utilizes EDC and NHS to promote the reaction of carboxylic acid modified QDs with amine modified ssDNA (forming either amide or ester bonds). However, these coupling strategies are reversible and highly dependent on pH, such that the conjugation yield is usually very low (Zhou, D. J. et al. Langmuir 24, 1659-1664 (2008)). “Click” chemistry could be used to generate 100% conjugation yield, however, a Cu catalyst is generally needed and Cu ions annihilate QD fluorescence (Cutler, et al., Nano Lett. 10, 1477-1480 (2010)). Recently, Kelley et al. reported a single-step synthetic route to produce chimeric, phosphorotiolated-phosphorodiester-DNA (ps-po-DNA) functionalized CdTe QDs (core only) in water (Ma, et al., Nature Nanotechnol. 4, 121-125 (2009); Tikhomirov, G. et al. Nature Nanotechnol. 6, 485-490 (2011)). The ssDNA contained a QD-binding domain with five or more consecutive ps modifications in the backbone, and a DNA-binding domain with a standard po backbone. Here, the multivalent interaction between the ps-domain and the QD surface greatly increases the binding affinity. Nevertheless, all of the previously described methods entail merely ‘adhering’ the oligonucleotides to the QD surface, and none fulfill all of the five requirements for robust and highly fluorescent oligonucleotide functionalized QDs.

As described herein, a new strategy to achieve robust DNA functionalized core/shell QDs that satisfy all of the five requirements for DNA-directed self-assembly is reported. The schematic shown in FIG. 1 illustrates the overall process of QD functionalization and subsequent DNA-origami directed assembly of the QD-DNA conjugates. This strategy takes advantage of chimeric ps-po-ssDNA strands that are directly inserted within a QD shell (thick CdS or ZnS shell) during synthesis over the core. This synthetic route results in core/shell QD—DNA conjugates that are chemically, photonically and colloidally stable, and highly fluorescent (PL quantum yields up to 70%), for a wide range of semiconductor materials with tunable fluorescent emissions spanning from UV to NIR (360 to 800 nm). As described herein, the organization of these QD—DNA conjugates by complementary base pairing to triangle and rectangular shaped DNA origami structures is further demonstrated.

The synthesis proceeded as follows: first, water-soluble, mercaptopropionic acid (MPA)-capped CdTe QDs cores were encapsulated by thick CdS shells in the presence of ps-po-ssDNA (details in FIGS. S1-S8). The magic size, MPA-capped CdTe nanocrystals (1.6 nm with PL peak at 480 nm) were synthesized following the methods outlined in Deng, Z. T. et al., J Am. Chem. Soc. 132, 5592-5593 (2010). In a typical reaction, an aliquot of CdTe core QDs was purified and re-dissolved in 100 μL, of nanopure water. A prescribed amount of Cd²⁺-MPA complex (serving as both the Cd²⁺ and S²⁻ precursors for CdS shell growth) and ps-po-ssDNA (the surface ligand) were added to the core mixture. The ps-po-ssDNA oligonucleotides (5′-G5-ps-T28-3′) contain a stretch of five consecutive guanine residues, followed by five consecutive ps backbone modifications and 28 unmodified thymine residues linked by conventional phosphodiester bonds. The pH of the mixture was adjusted to 12 and subsequently heated at 90° C. for 70 minutes. During this time, the Cd²⁺-MPA complex slowly decomposes and a CdS shell of particular thickness surrounds the CdTe core. The 5 sulfur atoms in the ps domain ‘insert’ into the CdS shell during its formation, while most (if not all) of the poly T domain extends away from the surface of the shell making it available for hybridization to complementary DNA within the underlying DNA nanostructure. As previously reported, at this relatively mild temperature the monolayer-by-monolayer formation of the CdS shell is fully controlled by the slow decomposition of the Cd²⁺-MPA complex (Deng, Z. T. et al., J. Am. Chem. Soc. 132, 5592-5593 (2010)). Here, the shell thickness is directed by the total reaction time. It was observed that 7 CdS shell monolayers are formed in 70 minutes, thus, the estimated synthesis time is 10 minutes/monolayer. Considering the relatively slow rate of growth, the S atoms in the ps-domain of the oligonucleotides have ample opportunity to bond to the Cd atoms and are readily incorporated into the CdS shell.

The resulting core/shell CdTe/CdS QD—DNA conjugates have an estimated diameter of 6.5 nm (7 CdS shell monolayers), with band-edge emission maxima at 672 nm and PL quantum yield of 70%. The observed ˜200 nm red shift of the emission peak is assigned to type-II QDs, where the energy bandgaps of the CdTe core and CdS shells are offset and the photo-generated electron-hole pair is separated across the core/shell barrier (Deng, Z. T. et al., J Am. Chem. Soc. 132, 5592-5593 (2010)). The TEM and HRTEM images reveal that the QD—DNA conjugates are monodispersed, single crystalline particles (FIG. 2a). The purified thick-shell QD—DNA conjugates are stable in a variety of buffer conditions, including 1× PBS buffer with a pH of 4.0, 7.0 and 10.0; 1× TAE-Mg²⁺ buffer; 1× TBE Mg²⁺ buffer; and 10× TAE-Mg²⁺ buffer (125 mM of Mg²⁺) (FIG. 2c).

These CdTe/CdS QD-DNA particles were subsequently assembled at precise positions on DNA origami structures via hybridization to complementary poly A capture probes extended from the surface of the origami (3 capture probes/1 QD—DNA). The organization of two or three QD—DNA conjugates on triangular and rectangular DNA origami was demonstrated, as shown in FIG. 2d-h. The self-assembled structures were evaluated by atomic force microscopy (AFM) and high-angle annular dark-field scanning transmission electron microscopy (HAADF-STEM) imaging techniques. The images in FIG. 2d-e confirm that over 95% of the triangle DNA origami structures display three QDs, one on each arm, as prescribed by the design (design details and additional images can be found in the supporting information FIGS. S22-S23). Meanwhile, 90% of the rectangular origami structures display two QD—DNA conjugates, one each at opposite corners, as directed by the design scheme (FIG. 2f-g). The height profile of the AFM images reveals that the size of the QD—DNA conjugates fall into a narrow range (6-7 nm).

Thick shell CdSe/CdS QD-DNA conjugates that contained 20 CdS monolayers were further synthesized. As reported by Hollingsworth et al. and Dubertret et al., “giant”-shell or thick-shell QDs are more chemically stable and exhibit reduced blinking behavior at the single particle level (Chen, Y. et al., J. Am. Chem. Soc. 130, 5026-5027 (2008); Mahler, B. et al., Nature Mater. 7, 659-664 (2008)). Thick-shell QDs have been achieved by the successive ionic layer adsorption and reaction (SILAR) method (Chen, Y. et al., J. Am. Chem. Soc. 130, 5026-5027 (2008); Mahler, B. et al., Nature Mater. 7, 659-664 (2008)) which requires high temperatures (240° C.) and a tedious growth process in organic solvent. Here, a new method to achieve thick-shell CdSe/20 CdS QDs at lower temperatures (90° C.) in aqueous solution was developed. More significantly, the ssDNA within the shell itself during the encapsulation process was incorporated. First oleic acid (OLA) capped CdSe core QDs (6 nm diameter) were synthesized in paraffin liquid at 320° C. (FIG. 3a&d and details in FIGS. S9-S16). The oleic acid capped CdSe cores had a diameter of approximately 6.0 nm and fluorescence emission at 650 nm. Next, 9 CdS shell monolayers were deposited on the CdSe core in aqueous solution at 90° C. with MPA serving as the capping ligand. After the ligand exchange and CdS shell growth, the tetrahedral shaped MPA-capped QD exhibited emission at 660 nm. The approximate length of these tetrahedral shaped QDs was 12 nm (FIG. 3b,e). Finally, the additional shells were incorporated on the QDs in the presence of ps-po-ssDNA. The resulting DNA oligonucleotide functionalized thick-shell CdSe/CdS QDs displayed emission at 663 nm. The length of the QD—DNA conjugates increased to ˜18 nm (FIG. 3c&f). The relatively small red shift in emission wavelength that occurred upon shell growth is because the CdSe/CdS core/shell QDs with large core size (6 nm diameter) are type I, where the narrow bandgap of CdSe falls completely within that of CdS. However, when small CdSe core QDs (3 nm diameter) with a thick-shell were encapsulated, a 70 nm PL red shift (from 565 to 635 nm, see FIG. S9) was observed; this is due to a strain induced PL shift, as reported by Nie et al. (Smith, et al., Nature Nanotechnol. 4, 56-63 (2009)).

As described herein, it has been demonstrated that these robust, thick shell QD—DNA conjugates are readily organized by addressable DNA origami structures to form, discrete, well-ordered nanoarchitectures. In addition, DNA origami structures are an ideal platform to confirm the successful functionalization of the QDs. As demonstrated in FIG. 3j, energy dispersive X-ray spectroscopy (EDS) of the self-assembled origami nanostructures reveals the presence of cadmium, selenium, sulfur, phosphorus from the CdSe/20 CdS QD-DNA conjugates, and uranium from the negative staining agent used to enhance the contrast of the DNA structures for TEM imaging (FIG. 3j).

As described herein, this strategy is quite versatile and can be applied to QDs composed of other semiconductor materials. For example, it was demonstrated that a ZnS shell can be deposited on a variety of different core materials using the same strategy. Using a water-soluble ZnSe core, core/shell ZnSe/ZnS QD—DNA conjugates (4 ZnS shell monolayers) that displayed UV emission at 360 nm (FIG. 4 and details in FIGS. S17-S21) were produced. Using oleic acid capped CdS or quaternary alloyed ZnCdSSe QD core materials (Deng, et al., J Am. Chem. Soc. 131, 17744-17745 (2009)), core/shell CdS/ZnS-DNA conjugates (4 ZnS shell monolayers) with blue emission at 425 nm and ZnCdSSe/ZnS QD-DNA conjugates (4 ZnS shell monolayers) with green emission at 510 nm, respectively, were synthesized. Further, core/shell CdTe/ZnS QD-DNA particles (4 ZnS shell monolayers) with yellow emission at 555 nm were produced. Finally, magic-sized CdTe core QDs were used to synthesize a series of DNA functionalized QDs, including: core/shell CdTe/CdS (2 CdS shell monolayers), CdTe/CdS (4 CdS shell monolayers), and thick-shell CdTe/CdS (10 or 13 CdS shell monolayers) QDs in the orange, red, and near infrared with emission maxima at 575, 610, 740, and 800 nm, respectively. As shown in FIG. 4, the organization of each of these QD-DNA conjugates by triangular DNA origami was demonstrated. The height profiles of the particles obtained from AFM cross-sections correspond well to the diameters measured using TEM imaging.

Given that CdS and ZnS are wide band gap semiconductor materials that are generally used in QD shells, it is reasonable to expect that many other core/thick-shell

QDs can be synthesized with various core compositions, such as binary PbS, InP, InAs QDs for IR emission, doped ZnSe:Mn QDs, ternary alloyed CuInSe or ZnCdSe QDs, and quaternary alloyed CuInSSe, etc., all of which should be compatible with the in-situ oligonucleotide functionalization strategy.

In summary, a simple and efficient method to synthesize robust QD-DNA conjugates that can withstand the conditions necessary for DNA-directed assembly has been developed. In contrast to QD functionalization strategies in which the DNA ligands are simply ‘adhered’ to the QD surface, ‘incorporation’ of the DNA within the shell material themselves was achieved, thus providing a higher level of stabilization. It has been demonstrated that this strategy can be used with a wide variety of semiconductor materials that display fluorescent emission spanning from UV-Vis to NIR. It has also been demonstrated that discrete numbers of QD—DNA conjugates can be organized by DNA origami nanostructures, an essential component of hierarchical nanoparticle assembly efforts. This work will facilitate the construction of discrete, multicomponent semiconductor or semiconductor-metal hybrid nanostructures for energy, nanophotonics, and biosensing applications.

### Materials and Methods

### Part 1. Chemicals, Buffers and Characterization Details

Chemicals: Cadmium nitrate tetrahydrate (Cd(NO₃)₂-4H₂O, 99.8%), Zinc nitrate tetrahydrate (Zn(NO₃)₂·4H₂O, 99.8%), Zinc oxide (ZnO, 99.9%, powder <5 micron), Cadmium oxide (CdO, 99.99+%, powder), Tellurium (Te, powder, −200 mesh, ≧99%, powder), Selenium (Se, powder, <100 mesh, 99.99%), Sulfur (S, 99.998% powder), paraffin liquid (CₙH₂ₙ₊₂, n=16-22), oleic acid (OLA, CH₃(CH₂)₇CH═CH(CH₂)₇COOH, 90%), 2-ethylhexanoic acid (EHA, CH₃(CH₂)₃CH(C₂H₅)COOH, 99+%), Thiourea (NH₂CSNH₂, ≧99.0%), Sodium borohydride (NaBH₄, powder, ≧99%), 3-Mercaptopropionic acid (HSCH₂CH₂CO₂H, ≧99%), isopropyl alcohol (IPA, 99%), hexane (≧95%), methanol (≧99.5%), Rhodamine 6G (QY=95% in ethanol), and Rhodamine 101 (λem=589 nm, QY=100% in ethanol+0.01 HCl), were purchased from Sigma-Aldrich and used without further purification. M13mp18 single stranded DNA was purchased from New England Biolabs and was also used without further treatment. All unmodified helper strands were purchased from Integrated DNA Technologies, Inc. (IDT, www.idtdna.com) in 96-well plate format, suspended in nanopure water (H₂O, with resistivity up to 18.2 MΩ·cm) and used without further purification. All modified helper strands were purchased from IDT and purified by denaturing PAGE gel electrophoresis. Phosphorothiolated backbone modified ps-po-chimeric ssDNA strands were purchased from IDT and used without purification.

Buffers: the buffers used in this study are:

1×PBS: 150 mM NaCl, 0.1 mM EDTA, 20 mM sodium phosphate, pH 4.0, 7.0, 10.0

1×TAE/Mg²⁺: 40 mM Tris acetate, 2 mM EDTA, and 12.5 mM magnesium acetate, pH 8.0.

1×TBE/Mg²⁺: 50 mM Tris, 100 mM Borate, 10 mM EDTA, pH 8.2.

Characterization: Ultraviolet-Visible (UV-Vis) absorption spectra were recorded at room temperature with a JASCO-V670 spectrophotometer. Photoluminescence (PL) spectra were measured at room temperature using a NanoLog spectrometer manufactured by HORIBA Jobin Yvon equipped with a thermoelectric cooled PMT (R928 in the range 200 nm to 850 nm). Atomic force microscopy (AFM) was performed using a Veeco 8 AFM in tapping in air mode. High-resolution transmission electron microscopy (HRTEM), high angle annular dark field scanning transmission electron microscopy (HAADF-STEM), and energy dispersive X-ray spectroscopy (EDS) were performed on a JEOL JEM 2010F electron microscope operating at 200 kV.

### Part 2. Oligonucleotide Functionalized CdTe/CdS QDs

2.1 Synthesis of 1.6 nm CdTe core QDs: CdTe core QDs with 1.6 nm diameter were synthesized according to Deng, Z. T. et al., Journal of the American Chemical Society 132, 5592-5593 (2010). A freshly prepared NaHTe solution (the source of Te, 1.0 mol/L, 10 μL) was injected through a syringe into an N₂-saturated Cd(NO₃)₂ solution (the source of Cd, 0.005 mol/L, 50 mL) at room temperature (20° C.) in the presence of 3-mercaptopropionic acid (MPA, 37 μL) as a stabilizing agent. The pH was tuned to 12.2 by adding NaOH (1 M). The molar ratio of Cd²⁺/MPA/NaHTe in the mixture was fixed at 1:1.7:0.04. The solution was subsequently aged at 4° C. and magic-sized CdTe clusters with photoluminescence emission peak at 480 nm were formed overnight. The diameter of the resulting CdTe QDs was ˜1.6 nm. These small QDs were purified by adding IPA (1:1 in volume ratio), followed by centrifugation at 15,000 rpm for 15 minutes and were subsequently re-dispersed in DI water. In some cases, the crude, unpurified CdTe QD solutions were also used directly as the stock solution for the next step shell growth. Both pure and impure solutions were used as the cores for synthesizing the oligonucleotides conjugated CdTe/CdS core/shell QDs.

2.2 Oligonucleotide functionalized CdTe/CdS core/shell QDs: The above precipitated 1.6 nm CdTe QDs (from 100 μL stock solution) were re-suspended in 100 μL of nano-pure water. The concentration of the core CdTe QDs and amount of additional shell precursor to obtain specific shell thicknesses were calculated following a reported method (Yu, et al., Chem. Mater. 15, 2854-2860 (2003); Smith, et al., Nature Nanotechnol. 4, 56-63 (2009)). For a typical experiment to synthesize CdTe/4 CdS core/shell QDs with 1.6 nm CdTe core diameter (0.25 nM in 100 μL, DI water), 4.5 μL Cd²⁺ stock solution (25 mM) and 9.0 μL MPA stock solution (25 mM) were combined with the core, vortexed and gently sonicated in a 1.5 mL plastic tube. Next, 50 μL of 5′-TTTTTTTTTTTTTTTTTTTTTTTTTTTTG*G*G*G*G* G -3′ oligonucleotide stock solution (100 nM) was also added and gently vortexed. The molar ratio of QD: oligonucleotide was approximately 1:200. The pH was tuned to 12.2 by adding NaOH (1M). The reaction mixture was placed on a heating block at 90° C. for 40 minutes, and then cooled down by submerging the tube in a water bath at room temperature. The reacted solution was loaded into a 0.5 mL Amicon filter (MWCO 30 KDa), 250 μL, DI water was added to the filter, and the sample was subjected to centrifugation at 7000 rpm for 3 minutes. The washing (each washing was performed with 350 μL of DI water) and centrifugation steps were repeated four times. This ultrafiltration process removed the free DNA and unreacted precursor from the QDs. If buffer exchange with DI water is desired, 350 μL of 1XTA buffer, rather than DI water, could be added before and after the centrifugation. The final sample is highly fluorescent and stable in buffer or in DI water.

It was determined that the un-purified crude CdTe QD cores could also be used for synthesis of oligonucleotide functionalized CdTe/CdS core/shell QDs. For a typical synthesis, 10 μL of crude 1.6 nm CdTe core stock solution (0.25 nM) was added to a 1.5 mL plastic tube. Then 2.5 μL of Cd²⁺ stock solution (25 mM) and 5.6 μL of MPA stock solution (25 mM) was added to the core, vortexed and gently sonicated. Next, 50 μL of 5′-TTTTTTTTTTTTTTTTTTTTTTTT TTTTG*G*G*G*G*G -3′ oligonucleotide stock solution (100 nM) was added and gently vortexed. The molar ratio of QD:oligonucleotide was about 1:200. The pH was tuned to 12.2 by adding NaOH (1M). Finally, the reacted solution was heated and purified as before.

### Photoluminescence Quantum Yield Measurements

A cross-calibrated method to measure the quantum yield of the as-synthesized quantum dots was used (http://www.horiba.com/us/en/scientific/products/fluorescence-spectroscopy/application-notes/quantum-yields/). For orange and red emission QDs, their PL quantum yields were obtained by comparison to a standard Rhodamine 101 reference dye (QY=100% in ethanol+0.01 HCl). The standard dye was cross-calibrated by referencing to Rhodamine 6G (QY=95% in ethanol). The PL quantum yield was calculated using the following equation:

$$ϕ = {ϕ}^{′} ( \frac{I}{{I}^{′}} ) ( \frac{{A}^{′}}{A} ) {( \frac{n}{{n}^{′}} )}^{2}$$

where φ and φ′ are the PL QY for the sample and standard, respectively; I (sample) and I′ (standard) are the integrated emission peak areas at a given wavelength; A (sample) and A′ (standard) are the absorption intensities at the same wavelength used for PL excitation; n (sample) and n′ (standard) are the refractive indices of the solvents.

### Part 3. Oligonucleotide Functionalized CdSe/20 CdS QDs

Synthesis of oleic acid (OLA) capped CdSe QDs (Deng, et al., Journal of Physical Chemistry B 109, 16671-16675 (2005)): Cd²⁺-OLA complex precursor solution was prepared by adding 7.5 mmol CdO into a 100 mL flask containing 10 mL paraffin liquid and 15 mL oleic acid. The mixture was heated to 100° C., degassed under 100 mtorr pressure for 30 minutes, filled with N2, and further heated to 200° C. to form a clear Cd²⁺ precursor solution. Then, Se precursor solution was prepared in a separate flask, where 0.30 mmol of Se powder was mixed with 15 mL paraffin liquid, degassed for 30 minutes, filled with N₂, and heated to 250 or 320° C. Next, 1 mL Cd²⁺-OLA complex precursor solution was quickly injected to the flask containing the above mixture. The molar ratio of Cd:Se in the reaction mixture was 1:1. The mixture was maintained at 250° C. for 10 minutes (for 3 nm CdSe QDs) or at 320 ° C. for 10 minutes (for 6 nm CdSe QDs) with continuous stirring. A number of aliquots were collected in test tubes containing cold hexane to further quench QD growth at different intervals. The samples were purified by centrifugation (13,000 rpm for 30 minutes) several times after being precipitated with IPA and methanol. The final products were dispersed in hexane.

### Oligonucleotide Functionalized Thick-Shell CdSe/CdS QDs:

First, ligand exchange with MPA was performed to make the CdSe core QDs water soluble: The oleic acid capped CdSe QDs, with emission at 650 nm, were purified as described above and dissolved in hexane (200 μL). Formamide (100 μL) mixed with 5 μL of 25 mM MPA solution was added. The mixture was vortexed and sonicated for 15 minutes to allow for ligand exchange in which MPA displaces OLA on the QD surface to form Cd-S bonds (rather than Cd—O bonds). After the ligand exchange, the MPA-capped QDs are soluble in the polar solvent, thus, can transfer from the non-polar hexane phase into the polar formamide phase. After settling, the upper hexane layer was removed, and the formamide layer was mixed with 1:1 IPA and centrifuged at 15K rpm for 10 minutes.

The purified QDs were re-dissolved in DI water. The concentration of the QDs was determined according reported methods.

Next, 9 CdS shell monolayers were deposited on the 6 nm CdSe core. For a typical experiment, the water-soluble CdSe QD solution (with absorption of 0.0031 at 625 nm) was prepared in 100 μL of DI-water. Then, 11 μL of Cd²⁺ stock solution (25 mM) and 22 μL, of MPA stock solution (25 mM) were added, vortexed and gently sonicated in a 1.5 mL plastic tube. The pH was tuned to 12.2 by adding NaOH (1M). The mixture was heated on a heating block at 90° C. for 90 minutes. The product was cooled down by submerging the tube in a water bath at room temperature. The sample was loaded into a 0.5 mL Amicon filter (MWCO 100 KDa), and filtered in the same way as the CdTe/CdS QDs to remove the unreacted precursor.

Finally, 11 CdS shell monolayers were deposited on the 6 nm CdSe core. For a typical experiment, the water soluble CdSe QD solution (with absorption value of 0.0031 at 625 nm) was prepared in 100 μL of DI-water by gentle sonication. Then, 40 μL of Cd²⁺ stock solution (25 mM) and 80 μL of MPA stock solution (25 mM) were added, vortexed and gently sonicated in a 1.5 mL plastic tube. Then 100 μL 5′-TTTTTTTTTTTTTTTTTTTTTTTTTTTTG*G*G*G*G* G*G*G*G*G*G-3′ oligonucleotide stock solution (100 nM) was added and gently vortexed. The molar ratio of QD:oligonucleotide was approximately 1:8.3. The pH was tuned to 12.2 by adding NaOH (1 M). The mixture was heated on the heating block at 90° C. for 110 minutes. Then, the product was cooled down by submerging the tube in a water bath at room temperature. The sample was loaded into a 0.5 mL Amicon filter (MWCO 100KDa), and filtered in the same way as for the CdTe/CdS QDs to remove the unreacted precursor from the QDs. If buffer exchange with DI water is desired, 350 μL of 1XTA buffer, rather than DI water could be added before and after the centrifugation. The final sample is fluorescent and stable in buffer or in DI water.

### Part 4. Oligonucleotide Functionalization of Other Core/Shell QDs

### 4.1. Oligonucleotide Functionalization of CdTe/ZnS QDs

The precipitated 1.6 nm core CdTe QDs (from 100 μL stock solution) were re-suspended in 100 μL of nano-pure water as described in Part 2 above. For oligonucleotide functionalized CdTe/4 ZnS core/shell QDs synthesis, 4.5 μL of Zn²⁺ stock solution (25 mM) and 9.0 μL of MPA stock solution (25 mM) were added to the 1.6 nm CdTe core (0.25 nM in 100 μL DI water) solution in a 1.5 mL plastic tube, vortexed and gently sonicated. Then, 504 of 5′-TTTTTT TTTTTTTTTTTTTTTTTTTTTTG*G*G*G*G*G-3′ oligonucleotide stock solution (100 nM) was added and gently vortexed. The molar ratio of QD:oligonucleotide was approximately 1:200. The pH was tuned to 12.2 by adding NaOH (1M). The reaction mixture was placed on the heating block at 90° C. for 40 minutes, and then cooled down at room temperature. The 0.5 mL Amicon filter (MWCO 30 KDa) washing step is the same as for the CdTe/CdS QDs described above. The final samples are highly fluorescent and stable in buffer or in DI water.

### 4.2 Oligonucleotids Functionalization of ZnSe/ZnS QDs

Synthesis of MPA-capped ZnSe QD core in aqueous solution (Deng, Z. T. et al., Langmuir 25, 434-442 (2009)) proceeded as follows: a freshly prepared NaHSe solution (the source of Se, 1.0 mol/L, 10 μL) was injected through a syringe to N₂-saturated Zn(NO₃)₂ solution in water (the source of Zn, 0.005 mol/L, 50 mL) at room temperature (20° C.) in the presence of 3-mercaptopropionic acid (MPA, 37 pi) as a stabilizing agent. The pH was tuned to 11.5 by adding NaOH (1M). The molar ratio of Zn²⁺/MPA/NaHSe in the mixture was fixed at 1:1.7:0.1. Then the solution was aged at 4° C. and magic-sized ZnSe clusters with absorption peak at 290 nm (no detectable emission) were formed overnight. These ZnSe core QDs were purified by adding IPA and centrifugation at 15,000 rpm for 15 minutes and used as cores for the synthesis of ZnSe/ZnS core/shell QDs.

Oligonucleotide functionalization of ZnSe/4ZnS QDs: The synthesis is similar to that of the CdTe/ZnS core/shell nanocrystals. For a typical synthesis, 4.5 μL of Zn²⁺ stock solution (25 mM) and 9.0 μL of MPA stock solution (25 mM), were added to the ZnSe core (0.25 nM) QDs in 100 μL of DI water, vortexed and gently sonicated. Then, 50 μL of 5′-TTTTTTTTTTTTTTT TTTTTTTTTTTTTG*G*G*G*G*G -3′ oligonucleotide stock solution (100 nM) was added and gently vortexed. The molar ratio of QD:oligonucleotide was approximately 1:200. The pH was tuned to 12.2 by adding NaOH (1M). The reaction mixture was placed on a heating block at 90° C. for 40 minutes, and then cooled down at room temperature. The 0.5 mL Amicon filter (MWCO 30 KDa) washing step is the same as for the CdTe/CdS QDs described above. The final sample is highly fluorescent and stable in buffer or in DI water.

### 4.3 Oligonucleotide Functionalization of CdS/ZnS QDs

Synthesis of Oleic acid capped CdS QDs (Deng, et al., Journal of the American Chemical Society 131, 17744-17745 (2009)): Cd²⁺-complex precursor solution was prepared by adding 7.5 mmol CdO to a 100 mL flask containing 10 mL of paraffin liquid and 15 mL of oleic acid. The mixture was heated to 100° C., degassed under 100 mtorr pressure for 30 minutes, filled with N₂, and further heated to 200° C. to form a clear solution of Cd²⁺ precursor. Then, S precursor solution was prepared in a separate flask, where 0.3 mmol of S powder was mixed with 15 mL of paraffin liquid, degassed for 30 minutes, filled with N₂, and heated to 220° C. At this temperature, 1 mL of Cd²⁺ precursor solution was quickly injected to the flask containing the above mixture. The molar ratio of Cd:S in the reaction mixture was 1:1. The mixture was then maintained at 220° C. with continuous stirring. A typical sample with emission at 410 nm was collected in a test tube containing 2 mL of cold hexane to further quench QD growth (10 minutes). The samples were purified by centrifugation (13,000 rpm for 30 minutes) several times after being precipitated with IPA and methanol.

First, ligand exchange with MPA was performed to render the CdS core QDs water soluble: The oleic acid capped CdS QDs with emission at 410 nm were purified as described above and dissolved in hexane (200 μL). Formamide (100 μL) mixed with 5 _{R}L of 25 mM MPA solution was added to the solution. The mixture was vortexed and sonicated for 15 minutes to allow for ligand exchange. After the ligand exchange, the MPA-capped QDs were soluble in the polar solvent, thus, transferred from the non-polar hexane phase into the polar formamide phase. After settling, the upper hexane layer was removed, and the formamide layer was mixed with 1:1 IPA, and centrifuged at 15 K rpm for 10 minutes. The purified QDs were re-dissolved in DI water.

Next, we further deposited 4 ZnS shell monolayers on the CdS core. For a typical experiment, the water soluble CdS QD solution (0.5 nM) was prepared in 100 μL DI-water by gentle sonication. Then, 4.5 μL of Zn²⁺ stock solution (25 mM) and 9 μL of MPA stock solution (25 mM) were added, vortexed and gently sonicated in a 1.5 mL plastic tube. Then 100 μL of 5′-TTT TTTTTTTTTTTTTTTTTTTTTTTTTG*G*G*G*G* G-3′ oligonucleotide stock solution (100 nM) was added and gently vortexed. The molar ratio of QD:oligonucleotide was approximately 1:200. The pH was tuned to 12.2 by adding NaOH (1M). The mixture was heated at on a heating block at 90° C. for 40 minutes. Then, the product was cooled down by submerging the tube in a water bath at room temperature. The 0.5 mL Amicon filter (MWCO 30 KDa) washing step is the same as for the CdTe/CdS QDs described above. The final sample is fluorescent and stable in buffer or in DI water.

### 4.4 Oligonucleotide Functionalization of ZnCdSSe/ZnS QDs

Oleic acid capped quaternary alloyed ZnCdSeS QDs (Deng, et al., Journal of the American Chemical Society 131, 17744-17745 (2009)): In a typical reaction for Zn_{0.60}Cd_{0.40}S_{0.33}Se_{0.66} QDs, Zn/Cd-complex precursor solution was prepared by adding 4.5 mmol of ZnO and 3.0 mmol of CdO (molar ratio of Zn:Cd is 3:2) into a 100 mL flask containing 10 mL of paraffin liquid, 10 mL of oleic acid, and 5 mL of 2-ethylhexanoic acid. The mixture was heated to 100° C., degassed under 100 mtorr pressure for 30 minutes, filled with N₂, and further heated to 200° C. to form a clear solution of Zn/Cd precursor. Then, S/Se precursor solution was prepared in a separate flask, where 0.10 mmol of S and 0.20 mmol of Se (molar ratio of S:Se is 1:2) were mixed with 15 mL paraffin liquid, degassed for 30 minutes, filled with N₂, and heated to 280 ° C. At this temperature, 1 mL of the Cd/Zn precursor solution was quickly injected to the flask containing the above mixture (the molar ratio of (Zn+Cd)/(S+Se)=1:1. The new mixture was then maintained at 280 ° C. with continuous stirring for 10 minutes. The samples were purified by centrifugation (13,000 rpm for 30 minutes) several times after being precipitated with IPA and methanol. The final products were dispersed in hexane.

Next, ligand exchange with MPA rendered the Zn_{0.6}Cd_{0.4}S_{0.33}Se_{0.66} core QDs water soluble in a similar manner as for the CdS QDs described above. Finally, 4 ZnS shell monolayers were deposited on the water soluble Zn_{0.6}Cd_{0.4}S_{0.33}Se_{0.66} core. For a typical experiment, a diluted QD solution (1 nM) was prepared in 100 μL DI-water by gentle sonication. Then, 4.5 μL of Cd²⁺stock solution (25 mM) and 9 μL of MPA stock solution (25 mM) were added, vortexed and gently sonicated in a 1.5 mL plastic tube. Then, 100 μL of 5′-TTTTTTTTTTTTTTTTTTTTT TTTTTTTG*G*G*G*G*G-3′ oligonucleotide stock solution (100 nM) was added and gently vortexed. The molar ratio of QD: oligonucleotide was approximately 1:100. The pH was tuned to 12.2 by adding NaOH (1M). The mixture was heated on a heating block at 90° C. for 40 minutes. Then, the product was cooled down by submerging the tube in a water bath at room temperature. The 0.5 mL Amicon filter (MWCO 30 KDa) washing step is the same as for the CdTe/CdS QDs described before. The final sample is fluorescent and stable in buffer or in DI water.

### Part 5. Synthesis of DNA Origami and Organization of QDs.

DNA origami is not only an excellent addressable substrate to achieve the self-assembled QD nanoarchitectures, but also acts as an ideal platform to confirm successful DNA functionalization of the QDs. We believe that this strategy is more straightforward and reliable than the previously reported QD-DNA-dye FRET method8 to characterize the ssDNA conjugated QDs.

Triangle and rectangular shaped DNA origami structures were formed following Rothemund's protocol (Rothemund, P. W. K., Nature 440, 297-302 (2006)). 3 nM single stranded M13 mp18 DNA was mixed with staple strands in a 1:5 ratio. To organize the QDs at specific positions on the origami platform, selected staple strands were modified with a poly A extension to serve as capture probes. 10 equivalents of the capture strands (rather than 5) were added to ensure they were incorporated into the origami structure. The origami was assembled in 1XTA-Mg buffer (40 mM Tris, 20 mM Acetic acid, and 12.5 mM Mg-acetate, pH 8.0) by cooling down slowly from 90° C. to 4° C. 100KDa MWCO Amicon filters were used to remove excess staple and capture strands from the sample.

For directed assembly of QDs on the DNA origami, first, the triangular or rectangular shaped DNA origami were assembled with the required number of staple strands and either 6 or 9 equivalents of the capture strands (each contained a 28 nucleotide single stranded overhang); here we used a 28 nucleotide Poly A sequence as the capture probe, extending from the origami surface, and a 28 nucleotide Poly T sequence within the po domain of the chimeric DNA functionalized on the core/shell QDs. This choice of sequence ensures a greater degree of freedom for strand hybridization, allowing sliding of the strands against one another and enough flexibility for all three capture strands in one cluster to simultaneously bind to a single QD.

Next, pre-formed DNA origami (various molar ratios) was added to the DNA-functionalized CdTe QDs in 1 ×TAE, Mg²⁺ buffer to form the desired structures. Additional 1 xTAE-Mg buffer was added to the sample to ensure that the solution was sufficiently dilute to reduce undesired crosslinking among the structures. Typically, 0.5 nM of triangle or rectangular origami was mixed with freshly prepared and purified QD-DNA conjugates in 1× TA-Mg buffer. The molar ratio of origami to QDs was 1:3 for triangular DNA origami and 1:2 for rectangular DNA origami. A typical total volume was 15 μL. The mixture was then annealed from 45° C. to 33° C. and recycled 20 times to complete the assembly process. The total self-assembly time is approximately 24 hours. High fidelity hybridization between capture strands and DNA strands on the core/shell QDs was verified by AFM and TEM of negatively stained samples. The position of the QDs reflect the design with nanometer precision.

### Part 6. Design of Triangular DNA Origami

This design reflects our intent to place three QDs of the same color, one on each of the three arms, on the triangle origami. A total of nine strands were modified at the 5′ end with a 28 nucleotide extension from the surface of the origami, with a complimentary DNA sequence on the surface of the QDs. In the schematic below, a purple bar is drawn on the 5′ end of the selected capture probes. The green circles are drawn to represent a diameter of 5 nm, illustrating how the probes are arranged into three clusters, each containing three probes that are intended to bind with the DNA strands on a single QD particle.

### Part 7. Design of Rectangular DNA Origami

This design reflects our intent to place two QDs of the same color in opposite corners of the asymmetric rectangle. A total of six strands were modified at the 5′ end with a 28 nucleotide extension from the surface of the origami, with a complimentary DNA sequence on the surface of the QDs. In the schematic below, a green bar is drawn on the 5′ end of the selected capture probes. The red circles are drawn to represent a diameter of 5 nm, illustrating how the probes are arranged into two clusters, each containing three probes that are intended to bind with the DNA strands on a single QD particle.

Although the foregoing specification fully discloses and enables the present invention, it is not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A DNA-functionalized conjugate comprising single-stranded DNA (ssDNA) strands conjugated to a semiconductor nanoparticle, wherein the nanoparticle provides fluorescent emissions.

2. The DNA-functionalized conjugate of claim 1, wherein the emission ranges from UV-vis to IR (360-800nm).

3. The DNA-functionalized conjugate of claim 1, wherein the nanoparticle is a zero-dimensional quantum dot, a one-dimensional quantum rod or wire, a two-dimensional quantum ribbon or sheet, or a three-dimensional structure.

4. The DNA-functionalized conjugate of claim 1, wherein the semiconductor nanoparticle comprises a binary material.

5. The DNA-functionalized conjugate of claim 1, wherein the binary material is CdTe, CdSe, CAS, ZnSe, PbS, PbSe, ternary alloyed ZnCdSe, CdSeS. CdPbTe, quaternary alloyed ZnCdSSe, Mn doped ZnSe or Mn doped ZnS.

6. The DNA-functionalized conjugate of claim 1, wherein the conjugation size is between 2 urn to 50 nm.

7. The DNA-functionalized conjugate of claim 1, wherein the DNA is a chimeric DNA comprising a phosphorothiolated phosphorodiester back bone.

8. The DNA-functionalized conjugate of claim 1, wherein the DNA-functionalized conjugate is stable and highly fluorescent.

9. The DNA-functionalized conjugate of claim 3, wherein the nanoparticle is a quantum dot comprising a core and a shell.

10. The DNA-functionalized conjugate of claim 9, wherein the shell material is binary CdS, ZnS, ZnSe, CdSe, or ternary ZnCdS.

11. The DNA-functionalized conjugate of claim 9, wherein the shell comprises 1-20 monolayers.

12. The DNA-functionalized conjugate of claim 9, wherein the DNA directly inserts into the shell.

13. The DNA-functionalized conjugate of claim 1, wherein the conjugate is stable over a pH range of 4-1.2.

14. The DNA-functionalized conjugate of claim 1, wherein the conjugate is stable at salt concentrations of greater than 10 nM Nd⁺ or Mg²⁺.

15. The DNA-functionalized conjugate of claim 1 wherein the conjugate exhibits bright fluorescence emission with quantum yields of up to 70%.

16. A DNA origami construct comprising the DNA functionalized conjugate of claim 1 and a DNA origami structure.

17. A method of synthesizing a DNA-functionalized conjugate, comprising encapsulating a nanoparticle core with a shell in the presence of ssDNA conjugate single-stranded DNA (ssDNA) to form a DNA-functionalized quantum dot.


SEQUENCE LISTING (430 sequences)

SEQ ID NO: 1 - DNA, 33 bp, Artificial Sequence
  Description of Artificial Sequence Synthetic oligonucleotide
gggggtttttttttttttttttttttttttttt

SEQ ID NO: 2 - DNA, 34 bp, Artificial Sequence
  Description of Artificial Sequence Synthetic oligonucleotide
ttttttttttttttttttttttttttttgggggg

SEQ ID NO: 3 - DNA, 39 bp, Artificial Sequence
  Description of Artificial Sequence Synthetic oligonucleotide
ttttttttttttttttttttttttttttggggggggggg

SEQ ID NO: 4 - DNA, 28 bp, Artificial Sequence
  Description of Artificial Sequence Synthetic oligonucleotide
aaaaaaaaaaaaaaaaaaaaaaaaaaaa

SEQ ID NO: 5 - DNA, 28 bp, Artificial Sequence
  Description of Artificial Sequence Synthetic oligonucleotide
tttttttttttttttttttttttttttt

SEQ ID NO: 6 - DNA, 64 bp, Artificial Sequence
  Description of Artificial Sequence Synthetic oligonucleotide
aaaaaaaaaaaaaaaaaaaatttttatcatcgttgaaagaggacagatggaagaaaaatctacg

SEQ ID NO: 7 - DNA, 64 bp, Artificial Sequence
  Description of Artificial Sequence Synthetic oligonucleotide
aaaaaaaaaaaaaaaaaaaattttttaataaaacgaactaaccgaactgaccaactcctgataa

SEQ ID NO: 8 - DNA, 64 bp, Artificial Sequence
  Description of Artificial Sequence Synthetic oligonucleotide
aaaaaaaaaaaaaaaaaaaattttaccagtcaggacgttggaacggtgtacagaccgaaacaaa

SEQ ID NO: 9 - DNA, 64 bp, Artificial Sequence
  Description of Artificial Sequence Synthetic oligonucleotide
aaaaaaaaaaaaaaaaaaaattttagcatgtatttcatcgtaggaatcaaacgattttttgttt

SEQ ID NO: 10 - DNA, 64 bp, Artificial Sequence
  Description of Artificial Sequence Synthetic oligonucleotide
aaaaaaaaaaaaaaaaaaaattttaacgtcaaaaatgaaaagcaagccgtttttatgaaaccaa

SEQ ID NO: 11 - DNA, 64 bp, Artificial Sequence
  Description of Artificial Sequence Synthetic oligonucleotide
aaaaaaaaaaaaaaaaaaaatttttcccaatccaaataagattaccgcgcccaataaataatat

SEQ ID NO: 12 - DNA, 64 bp, Artificial Sequence
  Description of Artificial Sequence Synthetic oligonucleotide
aaaaaaaaaaaaaaaaaaaattttgtttgcgtcacgctggtttgccccaagggagcccccgatt

SEQ ID NO: 13 - DNA, 64 bp, Artificial Sequence
  Description of Artificial Sequence Synthetic oligonucleotide
aaaaaaaaaaaaaaaaaaaatttttagagcttgacggggagttgcagcaagcggtcattgggcg

SEQ ID NO: 14 - DNA, 64 bp, Artificial Sequence
  Description of Artificial Sequence Synthetic oligonucleotide
aaaaaaaaaaaaaaaaaaaattttctaaatcggaaccctaagcaggcgaaaatccttcggccaa

SEQ ID NO: 15 - DNA, 32 bp, Artificial Sequence
  Description of Artificial Sequence Synthetic oligonucleotide
cggggtttcctcaagagaaggattttgaatta

SEQ ID NO: 16 - DNA, 32 bp, Artificial Sequence
  Description of Artificial Sequence Synthetic oligonucleotide
agcgtcatgtctctgaatttaccgactacctt

SEQ ID NO: 17 - DNA, 32 bp, Artificial Sequence
  Description of Artificial Sequence Synthetic oligonucleotide
ttcataatccccttattagcgtttttcttacc

SEQ ID NO: 18 - DNA, 32 bp, Artificial Sequence
  Description of Artificial Sequence Synthetic oligonucleotide
atggtttatgtcacaatcaatagatattaaac

SEQ ID NO: 19 - DNA, 40 bp, Artificial Sequence
  Description of Artificial Sequence Synthetic oligonucleotide
tttgatgattaagaggctgagacttgctcagtaccaggcg

SEQ ID NO: 20 - DNA, 32 bp, Artificial Sequence
  Description of Artificial Sequence Synthetic oligonucleotide
ccggaacccagaatggaaagcgcaacatggct

SEQ ID NO: 21 - DNA, 32 bp, Artificial Sequence
  Description of Artificial Sequence Synthetic oligonucleotide
aaagacaacattttcggtcatagccaaaatca

SEQ ID NO: 22 - DNA, 40 bp, Artificial Sequence
  Description of Artificial Sequence Synthetic oligonucleotide
gacgggagaattaactcggaataagtttatttccagcgcc

SEQ ID NO: 23 - DNA, 40 bp, Artificial Sequence
  Description of Artificial Sequence Synthetic oligonucleotide
gataagtgccgtcgagctgaaacatgaaagtatacaggag

SEQ ID NO: 24 - DNA, 32 bp, Artificial Sequence
  Description of Artificial Sequence Synthetic oligonucleotide
tgtactggaaatcctcattaaagcagagccac

SEQ ID NO: 25 - DNA, 32 bp, Artificial Sequence
  Description of Artificial Sequence Synthetic oligonucleotide
caccggaaagcgcgttttcatcggaagggcga

SEQ ID NO: 26 - DNA, 40 bp, Artificial Sequence
  Description of Artificial Sequence Synthetic oligonucleotide
cattcaacaaacgcaaagacaccagaacaccctgaacaaa

SEQ ID NO: 27 - DNA, 40 bp, Artificial Sequence
  Description of Artificial Sequence Synthetic oligonucleotide
tttaacggttcggaacctattattagggttgatataagta

SEQ ID NO: 28 - DNA, 32 bp, Artificial Sequence
  Description of Artificial Sequence Synthetic oligonucleotide
ctcagagcatattcacaaacaaattaataagt

SEQ ID NO: 29 - DNA, 32 bp, Artificial Sequence
  Description of Artificial Sequence Synthetic oligonucleotide
ggagggaatttagcgtcagactgtccgcctcc

SEQ ID NO: 30 - DNA, 40 bp, Artificial Sequence
  Description of Artificial Sequence Synthetic oligonucleotide
gtcagagggtaattgatggcaacatataaaagcgattgag

SEQ ID NO: 31 - DNA, 40 bp, Artificial Sequence
  Description of Artificial Sequence Synthetic oligonucleotide
tagcccggaataggtgaatgccccctgcctatggtcagtg

SEQ ID NO: 32 - DNA, 32 bp, Artificial Sequence
  Description of Artificial Sequence Synthetic oligonucleotide
ccttgagtcagacgattggccttgcgccaccc

SEQ ID NO: 33 - DNA, 32 bp, Artificial Sequence
  Description of Artificial Sequence Synthetic oligonucleotide
tcagaacccagaatcaagtttgccggtaaata

SEQ ID NO: 34 - DNA, 40 bp, Artificial Sequence
  Description of Artificial Sequence Synthetic oligonucleotide
ttgacggaaatacatacataaagggcgctaatatcagaga

SEQ ID NO: 35 - DNA, 35 bp, Artificial Sequence
  Description of Artificial Sequence Synthetic oligonucleotide
cagagccaggaggttgaggcaggtaacagtgcccg

SEQ ID NO: 36 - DNA, 32 bp, Artificial Sequence
  Description of Artificial Sequence Synthetic oligonucleotide
attaaaggccgtaatcagtagcgagccaccct

SEQ ID NO: 37 - DNA, 40 bp, Artificial Sequence
  Description of Artificial Sequence Synthetic oligonucleotide
gataacccacaagaatgttagcaaacgtagaaaattattc

SEQ ID NO: 38 - DNA, 24 bp, Artificial Sequence
  Description of Artificial Sequence Synthetic oligonucleotide
gccgccagcattgacaccaccctc

SEQ ID NO: 39 - DNA, 32 bp, Artificial Sequence
  Description of Artificial Sequence Synthetic oligonucleotide
agagccgcaccatcgatagcagcatgaattat

SEQ ID NO: 40 - DNA, 40 bp, Artificial Sequence
  Description of Artificial Sequence Synthetic oligonucleotide
caccgtcaccttattacgcagtattgagttaagcccaata

SEQ ID NO: 41 - DNA, 35 bp, Artificial Sequence
  Description of Artificial Sequence Synthetic oligonucleotide
agccatttaaacgtcaccaatgaacaccagaacca

SEQ ID NO: 42 - DNA, 40 bp, Artificial Sequence
  Description of Artificial Sequence Synthetic oligonucleotide
ataagagcaagaaacatggcatgattaagactccgacttg

SEQ ID NO: 43 - DNA, 24 bp, Artificial Sequence
  Description of Artificial Sequence Synthetic oligonucleotide
ccattagcaaggccgggggaatta

SEQ ID NO: 44 - DNA, 40 bp, Artificial Sequence
  Description of Artificial Sequence Synthetic oligonucleotide
gagccagcgaatacccaaaagaacatgaaatagcaatagc

SEQ ID NO: 45 - DNA, 43 bp, Artificial Sequence
  Description of Artificial Sequence Synthetic oligonucleotide
tatcttaccgaagcccaaacgcaataataacgaaaatcaccag

SEQ ID NO: 46 - DNA, 43 bp, Artificial Sequence
  Description of Artificial Sequence Synthetic oligonucleotide
cagaaggaaaccgaggtttttaagaaaagtaagcagatagccg

SEQ ID NO: 47 - DNA, 32 bp, Artificial Sequence
  Description of Artificial Sequence Synthetic oligonucleotide
ccttttttcatttaacaatttcataggattag

SEQ ID NO: 48 - DNA, 32 bp, Artificial Sequence
  Description of Artificial Sequence Synthetic oligonucleotide
tttaacctatcataggtctgagagttccagta

SEQ ID NO: 49 - DNA, 32 bp, Artificial Sequence
  Description of Artificial Sequence Synthetic oligonucleotide
agtataaaatatgcgttatacaaagccatctt

SEQ ID NO: 50 - DNA, 32 bp, Artificial Sequence
  Description of Artificial Sequence Synthetic oligonucleotide
caagtacctcattccaagaacgggaaattcat

SEQ ID NO: 51 - DNA, 32 bp, Artificial Sequence
  Description of Artificial Sequence Synthetic oligonucleotide
agagaataacataaaaacagggaagcgcatta

SEQ ID NO: 52 - DNA, 40 bp, Artificial Sequence
  Description of Artificial Sequence Synthetic oligonucleotide
aaaacaaaattaattaaatggaaacagtacattagtgaat

SEQ ID NO: 53 - DNA, 32 bp, Artificial Sequence
  Description of Artificial Sequence Synthetic oligonucleotide
ttatcaaaccggcttaggttgggtaagcctgt

SEQ ID NO: 54 - DNA, 32 bp, Artificial Sequence
  Description of Artificial Sequence Synthetic oligonucleotide
ttagtatcgccaacgctcaacagtcggctgtc

SEQ ID NO: 55 - DNA, 40 bp, Artificial Sequence
  Description of Artificial Sequence Synthetic oligonucleotide
tttccttagcactcatcgagaacaatagcagcctttacag

SEQ ID NO: 56 - DNA, 40 bp, Artificial Sequence
  Description of Artificial Sequence Synthetic oligonucleotide
agagtcaaaaatcaatatatgtgatgaaacaaacatcaag

SEQ ID NO: 57 - DNA, 32 bp, Artificial Sequence
  Description of Artificial Sequence Synthetic oligonucleotide
actagaaatatataactatatgtacgctgaga

SEQ ID NO: 58 - DNA, 32 bp, Artificial Sequence
  Description of Artificial Sequence Synthetic oligonucleotide
tcaataatagggcttaattgagaatcataatt

SEQ ID NO: 59 - DNA, 40 bp, Artificial Sequence
  Description of Artificial Sequence Synthetic oligonucleotide
aacgtcaaaaatgaaaagcaagccgtttttatgaaaccaa

SEQ ID NO: 60 - DNA, 40 bp, Artificial Sequence
  Description of Artificial Sequence Synthetic oligonucleotide
gagcaaaagaagatgagtgaataaccttgcttatagctta

SEQ ID NO: 61 - DNA, 32 bp, Artificial Sequence
  Description of Artificial Sequence Synthetic oligonucleotide
gattaagaaatgctgatgcaaatcagaataaa

SEQ ID NO: 62 - DNA, 32 bp, Artificial Sequence
  Description of Artificial Sequence Synthetic oligonucleotide
caccggaatcgccatatttaacaaaatttacg

SEQ ID NO: 63 - DNA, 40 bp, Artificial Sequence
  Description of Artificial Sequence Synthetic oligonucleotide
agcatgtatttcatcgtaggaatcaaacgattttttgttt

SEQ ID NO: 64 - DNA, 40 bp, Artificial Sequence
  Description of Artificial Sequence Synthetic oligonucleotide
acatagcgctgtaaatcgtcgctattcatttcaattacct

SEQ ID NO: 65 - DNA, 32 bp, Artificial Sequence
  Description of Artificial Sequence Synthetic oligonucleotide
gttaaatacaatcgcaagacaaagccttgaaa

SEQ ID NO: 66 - DNA, 32 bp, Artificial Sequence
  Description of Artificial Sequence Synthetic oligonucleotide
cccatcctcgccaacatgtaatttaataaggc

SEQ ID NO: 67 - DNA, 40 bp, Artificial Sequence
  Description of Artificial Sequence Synthetic oligonucleotide
tcccaatccaaataagattaccgcgcccaataaataatat

SEQ ID NO: 68 - DNA, 35 bp, Artificial Sequence
  Description of Artificial Sequence Synthetic oligonucleotide
tcccttagaataacgcgagaaaacttttaccgacc

SEQ ID NO: 69 - DNA, 32 bp, Artificial Sequence
  Description of Artificial Sequence Synthetic oligonucleotide
gtgtgataaggcagaggcattttcagtcctga

SEQ ID NO: 70 - DNA, 40 bp, Artificial Sequence
  Description of Artificial Sequence Synthetic oligonucleotide
acaagaaagcaagcaaatcagataacagccatattattta

SEQ ID NO: 71 - DNA, 24 bp, Artificial Sequence
  Description of Artificial Sequence Synthetic oligonucleotide
gtttgaaattcaaatatattttag

SEQ ID NO: 72 - DNA, 32 bp, Artificial Sequence
  Description of Artificial Sequence Synthetic oligonucleotide
aatagatagagccagtaataagagatttaatg

SEQ ID NO: 73 - DNA, 40 bp, Artificial Sequence
  Description of Artificial Sequence Synthetic oligonucleotide
gccagttacaaaataatagaaggcttatccggttatcaac

SEQ ID NO: 74 - DNA, 35 bp, Artificial Sequence
  Description of Artificial Sequence Synthetic oligonucleotide
ttctgacctaaaatataaagtaccgactgcagaac

SEQ ID NO: 75 - DNA, 40 bp, Artificial Sequence
  Description of Artificial Sequence Synthetic oligonucleotide
gcgcctgttattctaagaacgcgattccagagcctaattt

SEQ ID NO: 76 - DNA, 24 bp, Artificial Sequence
  Description of Artificial Sequence Synthetic oligonucleotide
tcagctaaaaaaggtaaagtaatt

SEQ ID NO: 77 - DNA, 40 bp, Artificial Sequence
  Description of Artificial Sequence Synthetic oligonucleotide
acgctaacgagcgtctggcgttttagcgaacccaacatgt

SEQ ID NO: 78 - DNA, 43 bp, Artificial Sequence
  Description of Artificial Sequence Synthetic oligonucleotide
acgacaataaatcccgacttgcgggagatcctgaatcttacca

SEQ ID NO: 79 - DNA, 43 bp, Artificial Sequence
  Description of Artificial Sequence Synthetic oligonucleotide
tgctattttgcacccagctacaattttgttttgaagccttaaa

SEQ ID NO: 80 - DNA, 32 bp, Artificial Sequence
  Description of Artificial Sequence Synthetic oligonucleotide
tcatatgtgtaatcgtaaaactagtcattttc

SEQ ID NO: 81 - DNA, 32 bp, Artificial Sequence
  Description of Artificial Sequence Synthetic oligonucleotide
gtgagaaaatgtgtaggtaaagatacaacttt

SEQ ID NO: 82 - DNA, 32 bp, Artificial Sequence
  Description of Artificial Sequence Synthetic oligonucleotide
ggcatcaaatttggggcgcgagctagttaaag

SEQ ID NO: 83 - DNA, 32 bp, Artificial Sequence
  Description of Artificial Sequence Synthetic oligonucleotide
ttcgagctaagacttcaaatatcgggaacgag

SEQ ID NO: 84 - DNA, 40 bp, Artificial Sequence
  Description of Artificial Sequence Synthetic oligonucleotide
acagtcaaagagaatcgatgaacgaccccggttgataatc

SEQ ID NO: 85 - DNA, 32 bp, Artificial Sequence
  Description of Artificial Sequence Synthetic oligonucleotide
atagtagtatgcaatgcctgagtaggccggag

SEQ ID NO: 86 - DNA, 32 bp, Artificial Sequence
  Description of Artificial Sequence Synthetic oligonucleotide
aaccagacgtttagctatattttcttctacta

SEQ ID NO: 87 - DNA, 40 bp, Artificial Sequence
  Description of Artificial Sequence Synthetic oligonucleotide
gaataccacattcaacttaagaggaagcccgatcaaagcg

SEQ ID NO: 88 - DNA, 40 bp, Artificial Sequence
  Description of Artificial Sequence Synthetic oligonucleotide
agaaaagccccaaaaagagtctggagcaaacaatcaccat

SEQ ID NO: 89 - DNA, 32 bp, Artificial Sequence
  Description of Artificial Sequence Synthetic oligonucleotide
caatatgaccctcatatattttaaagcattaa

SEQ ID NO: 90 - DNA, 32 bp, Artificial Sequence
  Description of Artificial Sequence Synthetic oligonucleotide
catccaataaatggtcaataacctcggaagca

SEQ ID NO: 91 - DNA, 40 bp, Artificial Sequence
  Description of Artificial Sequence Synthetic oligonucleotide
aactccaagattgcatcaaaaagataatgcagatacataa

SEQ ID NO: 92 - DNA, 40 bp, Artificial Sequence
  Description of Artificial Sequence Synthetic oligonucleotide
cgttctagtcaggtcattgcctgacaggaagattgtataa

SEQ ID NO: 93 - DNA, 32 bp, Artificial Sequence
  Description of Artificial Sequence Synthetic oligonucleotide
caggcaagataaaaatttttagaatattcaac

SEQ ID NO: 94 - DNA, 32 bp, Artificial Sequence
  Description of Artificial Sequence Synthetic oligonucleotide
gattagagattagatacatttcgcaaatcata

SEQ ID NO: 95 - DNA, 40 bp, Artificial Sequence
  Description of Artificial Sequence Synthetic oligonucleotide
cgccaaaaggaattacagtcagaagcaaagcgcaggtcag

SEQ ID NO: 96 - DNA, 40 bp, Artificial Sequence
  Description of Artificial Sequence Synthetic oligonucleotide
gcaaatatttaaattgagatctacaaaggctactgataaa

SEQ ID NO: 97 - DNA, 32 bp, Artificial Sequence
  Description of Artificial Sequence Synthetic oligonucleotide
ttaatgccttatttcaacgcaagggcaaagaa

SEQ ID NO: 98 - DNA, 32 bp, Artificial Sequence
  Description of Artificial Sequence Synthetic oligonucleotide
ttagcaaatagatttagtttgaccagtacctt

SEQ ID NO: 99 - DNA, 40 bp, Artificial Sequence
  Description of Artificial Sequence Synthetic oligonucleotide
taattgctttaccctgactattatgaggcatagtaagagc

SEQ ID NO: 100 - DNA, 35 bp, Artificial Sequence
  Description of Artificial Sequence Synthetic oligonucleotide
ataaagcctttgcgggagaagcctggagagggtag

SEQ ID NO: 101 - DNA, 32 bp, Artificial Sequence
  Description of Artificial Sequence Synthetic oligonucleotide
taagaggtcaattctgcgaacgagattaagca

SEQ ID NO: 102 - DNA, 40 bp, Artificial Sequence
  Description of Artificial Sequence Synthetic oligonucleotide
aacactatcataacccatcaaaaatcaggtctccttttga

SEQ ID NO: 103 - DNA, 24 bp, Artificial Sequence
  Description of Artificial Sequence Synthetic oligonucleotide
atgaccctgtaatacttcagagca

SEQ ID NO: 104 - DNA, 32 bp, Artificial Sequence
  Description of Artificial Sequence Synthetic oligonucleotide
taaagctatataacagttgattcccatttttg

SEQ ID NO: 105 - DNA, 40 bp, Artificial Sequence
  Description of Artificial Sequence Synthetic oligonucleotide
cggatggcacgagaatgaccataatcgtttaccagacgac

SEQ ID NO: 106 - DNA, 35 bp, Artificial Sequence
  Description of Artificial Sequence Synthetic oligonucleotide
taattgcttggaagtttcattccaaatcggttgta

SEQ ID NO: 107 - DNA, 40 bp, Artificial Sequence
  Description of Artificial Sequence Synthetic oligonucleotide
gataaaaaccaaaatattaaacagttcagaaattagagct

SEQ ID NO: 108 - DNA, 24 bp, Artificial Sequence
  Description of Artificial Sequence Synthetic oligonucleotide
actaaagtacggtgtcgaatataa

SEQ ID NO: 109 - DNA, 40 bp, Artificial Sequence
  Description of Artificial Sequence Synthetic oligonucleotide
tgctgtagatccccctcaaatgctgcgagaggcttttgca

SEQ ID NO: 110 - DNA, 43 bp, Artificial Sequence
  Description of Artificial Sequence Synthetic oligonucleotide
aaagaagttttgccagcataaatattcattgactcaacatgtt

SEQ ID NO: 111 - DNA, 43 bp, Artificial Sequence
  Description of Artificial Sequence Synthetic oligonucleotide
aatactgcggaatcgtagggggtaatagtaaaatgtttagact

SEQ ID NO: 112 - DNA, 32 bp, Artificial Sequence
  Description of Artificial Sequence Synthetic oligonucleotide
agggatagctcagagccaccaccccatgtcaa

SEQ ID NO: 113 - DNA, 32 bp, Artificial Sequence
  Description of Artificial Sequence Synthetic oligonucleotide
caacagtttatgggattttgctaatcaaaagg

SEQ ID NO: 114 - DNA, 32 bp, Artificial Sequence
  Description of Artificial Sequence Synthetic oligonucleotide
gccgctttgctgaggcttgcaggggaaaaggt

SEQ ID NO: 115 - DNA, 32 bp, Artificial Sequence
  Description of Artificial Sequence Synthetic oligonucleotide
gcgcagactccatgttacttagcccgttttaa

SEQ ID NO: 116 - DNA, 32 bp, Artificial Sequence
  Description of Artificial Sequence Synthetic oligonucleotide
acaggtagaaagattcatcagttgagatttag

SEQ ID NO: 117 - DNA, 40 bp, Artificial Sequence
  Description of Artificial Sequence Synthetic oligonucleotide
cctcagaaccgccacccaagcccaataggaacgtaaatga

SEQ ID NO: 118 - DNA, 32 bp, Artificial Sequence
  Description of Artificial Sequence Synthetic oligonucleotide
attttctgtcagcggagtgagaataccgatat

SEQ ID NO: 119 - DNA, 32 bp, Artificial Sequence
  Description of Artificial Sequence Synthetic oligonucleotide
attcggtctgcgggatcgtcacccgaaatccg

SEQ ID NO: 120 - DNA, 40 bp, Artificial Sequence
  Description of Artificial Sequence Synthetic oligonucleotide
cgacctgcggtcaatcataagggaacggaacaacattatt

SEQ ID NO: 121 - DNA, 40 bp, Artificial Sequence
  Description of Artificial Sequence Synthetic oligonucleotide
agacgttaccatgtaccgtaacacccctcagaaccgccac

SEQ ID NO: 122 - DNA, 32 bp, Artificial Sequence
  Description of Artificial Sequence Synthetic oligonucleotide
cacgcataagaaaggaacaactaagtctttcc

SEQ ID NO: 123 - DNA, 32 bp, Artificial Sequence
  Description of Artificial Sequence Synthetic oligonucleotide
attgtgtctcagcagcgaaagacaccatcgcc

SEQ ID NO: 124 - DNA, 40 bp, Artificial Sequence
  Description of Artificial Sequence Synthetic oligonucleotide
ttaataaaacgaactaaccgaactgaccaactcctgataa

SEQ ID NO: 125 - DNA, 40 bp, Artificial Sequence
  Description of Artificial Sequence Synthetic oligonucleotide
aggtttagtaccgccatgagtttcgtcaccaggatctaaa

SEQ ID NO: 126 - DNA, 32 bp, Artificial Sequence
  Description of Artificial Sequence Synthetic oligonucleotide
gttttgtcaggaattgcgaataatccgacaat

SEQ ID NO: 127 - DNA, 32 bp, Artificial Sequence
  Description of Artificial Sequence Synthetic oligonucleotide
gacaacaagcatcggaacgagggtgagatttg

SEQ ID NO: 128 - DNA, 40 bp, Artificial Sequence
  Description of Artificial Sequence Synthetic oligonucleotide
tatcatcgttgaaagaggacagatggaagaaaaatctacg

SEQ ID NO: 129 - DNA, 40 bp, Artificial Sequence
  Description of Artificial Sequence Synthetic oligonucleotide
agcgtaactacaaactacaacgcctatcaccgtactcagg

SEQ ID NO: 130 - DNA, 32 bp, Artificial Sequence
  Description of Artificial Sequence Synthetic oligonucleotide
tagttgcgaattttttcacgttgatcatagtt

SEQ ID NO: 131 - DNA, 32 bp, Artificial Sequence
  Description of Artificial Sequence Synthetic oligonucleotide
gtacaacgagcaacggctacagaggataccga

SEQ ID NO: 132 - DNA, 40 bp, Artificial Sequence
  Description of Artificial Sequence Synthetic oligonucleotide
accagtcaggacgttggaacggtgtacagaccgaaacaaa

SEQ ID NO: 133 - DNA, 35 bp, Artificial Sequence
  Description of Artificial Sequence Synthetic oligonucleotide
acagacagcccaaatctccaaaaaaaaatttctta

SEQ ID NO: 134 - DNA, 32 bp, Artificial Sequence
  Description of Artificial Sequence Synthetic oligonucleotide
aacagcttgctttgaggactaaagcgattata

SEQ ID NO: 135 - DNA, 40 bp, Artificial Sequence
  Description of Artificial Sequence Synthetic oligonucleotide
ccaagcgcaggcgcataggctggcagaactggctcattat

SEQ ID NO: 136 - DNA, 24 bp, Artificial Sequence
  Description of Artificial Sequence Synthetic oligonucleotide
cgaggtgaggctccaaaaggagcc

SEQ ID NO: 137 - DNA, 32 bp, Artificial Sequence
  Description of Artificial Sequence Synthetic oligonucleotide
acccccagactttttcatgaggaacttgcttt

SEQ ID NO: 138 - DNA, 40 bp, Artificial Sequence
  Description of Artificial Sequence Synthetic oligonucleotide
accttatgcgattttatgaccttcatcaagagcatctttg

SEQ ID NO: 139 - DNA, 35 bp, Artificial Sequence
  Description of Artificial Sequence Synthetic oligonucleotide
cggtttatcaggtttccattaaacgggaatacact

SEQ ID NO: 140 - DNA, 40 bp, Artificial Sequence
  Description of Artificial Sequence Synthetic oligonucleotide
aaaacacttaatcttgacaagaacttaatcattgtgaatt

SEQ ID NO: 141 - DNA, 24 bp, Artificial Sequence
  Description of Artificial Sequence Synthetic oligonucleotide
ggcaaaagtaaaatacgtaatgcc

SEQ ID NO: 142 - DNA, 40 bp, Artificial Sequence
  Description of Artificial Sequence Synthetic oligonucleotide
tggtttaatttcaactcggatattcattacccacgaaaga

SEQ ID NO: 143 - DNA, 43 bp, Artificial Sequence
  Description of Artificial Sequence Synthetic oligonucleotide
accaacctaaaaaatcaacgtaacaaataaattgggcttgaga

SEQ ID NO: 144 - DNA, 43 bp, Artificial Sequence
  Description of Artificial Sequence Synthetic oligonucleotide
cctgacgagaaacaccagaacgagtaggctgctcattcagtga

SEQ ID NO: 145 - DNA, 32 bp, Artificial Sequence
  Description of Artificial Sequence Synthetic oligonucleotide
tcgggagatatacagtaacagtacaaataatt

SEQ ID NO: 146 - DNA, 32 bp, Artificial Sequence
  Description of Artificial Sequence Synthetic oligonucleotide
cctgattaaaggagcggaattatctcggcctc

SEQ ID NO: 147 - DNA, 32 bp, Artificial Sequence
  Description of Artificial Sequence Synthetic oligonucleotide
gcaaatcacctcaatcaatatctgcaggtcga

SEQ ID NO: 148 - DNA, 32 bp, Artificial Sequence
  Description of Artificial Sequence Synthetic oligonucleotide
cgaccagtacattggcagattcacctgattgc

SEQ ID NO: 149 - DNA, 40 bp, Artificial Sequence
  Description of Artificial Sequence Synthetic oligonucleotide
tggcaatttttaacgtcagatgaaaacaataacggattcg

SEQ ID NO: 150 - DNA, 32 bp, Artificial Sequence
  Description of Artificial Sequence Synthetic oligonucleotide
aaggaattacaaagaaaccaccagtcagatga

SEQ ID NO: 151 - DNA, 32 bp, Artificial Sequence
  Description of Artificial Sequence Synthetic oligonucleotide
ggacattcacctcaaatatcaaacacagttga

SEQ ID NO: 152 - DNA, 40 bp, Artificial Sequence
  Description of Artificial Sequence Synthetic oligonucleotide
ttgacgagcacgtatactgaaatggattatttaataaaag

SEQ ID NO: 153 - DNA, 40 bp, Artificial Sequence
  Description of Artificial Sequence Synthetic oligonucleotide
cctgattgctttgaattgcgtagattttcaggcatcaata

SEQ ID NO: 154 - DNA, 32 bp, Artificial Sequence
  Description of Artificial Sequence Synthetic oligonucleotide
taatcctgattatcattttgcggagaggaagg

SEQ ID NO: 155 - DNA, 32 bp, Artificial Sequence
  Description of Artificial Sequence Synthetic oligonucleotide
ttatctaaagcatcaccttgctgatggccaac

SEQ ID NO: 156 - DNA, 40 bp, Artificial Sequence
  Description of Artificial Sequence Synthetic oligonucleotide
agagatagtttgacgctcaatcgtacgtgctttcctcgtt

SEQ ID NO: 157 - DNA, 40 bp, Artificial Sequence
  Description of Artificial Sequence Synthetic oligonucleotide
gattatacacagaaataaagaaataccaagttacaaaatc

SEQ ID NO: 158 - DNA, 32 bp, Artificial Sequence
  Description of Artificial Sequence Synthetic oligonucleotide
taggagcataaaagtttgagtaacattgtttg

SEQ ID NO: 159 - DNA, 32 bp, Artificial Sequence
  Description of Artificial Sequence Synthetic oligonucleotide
tgacctgacaaatgaaaaatctaaaatatctt

SEQ ID NO: 160 - DNA, 40 bp, Artificial Sequence
  Description of Artificial Sequence Synthetic oligonucleotide
agaatcagagcgggagatggaaatacctacataacccttc

SEQ ID NO: 161 - DNA, 40 bp, Artificial Sequence
  Description of Artificial Sequence Synthetic oligonucleotide
gcgcagaggcgaattaattatttgcacgtaaattctgaat

SEQ ID NO: 162 - DNA, 32 bp, Artificial Sequence
  Description of Artificial Sequence Synthetic oligonucleotide
aatggaagcgaacgttattaatttctaacaac

SEQ ID NO: 163 - DNA, 32 bp, Artificial Sequence
  Description of Artificial Sequence Synthetic oligonucleotide
taatagatcgctgagagccagcagaagcgtaa

SEQ ID NO: 164 - DNA, 40 bp, Artificial Sequence
  Description of Artificial Sequence Synthetic oligonucleotide
gaatacgtaacaggaaaaacgctcctaaacaggaggccga

SEQ ID NO: 165 - DNA, 35 bp, Artificial Sequence
  Description of Artificial Sequence Synthetic oligonucleotide
tcaatagatattaaatcctttgccggttagaacct

SEQ ID NO: 166 - DNA, 32 bp, Artificial Sequence
  Description of Artificial Sequence Synthetic oligonucleotide
caatatttgcctgcaacagtgccatagagccg

SEQ ID NO: 167 - DNA, 40 bp, Artificial Sequence
  Description of Artificial Sequence Synthetic oligonucleotide
ttaaagggattttagataccgccagccattgcggcacaga

SEQ ID NO: 168 - DNA, 24 bp, Artificial Sequence
  Description of Artificial Sequence Synthetic oligonucleotide
acaattcgacaactcgtaatacat

SEQ ID NO: 169 - DNA, 32 bp, Artificial Sequence
  Description of Artificial Sequence Synthetic oligonucleotide
ttgaggatggtcagtattaacaccttgaatgg

SEQ ID NO: 170 - DNA, 40 bp, Artificial Sequence
  Description of Artificial Sequence Synthetic oligonucleotide
ctattagtatatccagaacaatatcaggaacggtacgcca

SEQ ID NO: 171 - DNA, 35 bp, Artificial Sequence
  Description of Artificial Sequence Synthetic oligonucleotide
cgcgaactaaaacagaggtgaggcttagaagtatt

SEQ ID NO: 172 - DNA, 40 bp, Artificial Sequence
  Description of Artificial Sequence Synthetic oligonucleotide
gaatcctgagaagtgtatcggccttgctggtactttaatg

SEQ ID NO: 173 - DNA, 24 bp, Artificial Sequence
  Description of Artificial Sequence Synthetic oligonucleotide
accaccagcagaagatgatagccc

SEQ ID NO: 174 - DNA, 40 bp, Artificial Sequence
  Description of Artificial Sequence Synthetic oligonucleotide
taaaacattagaagaactcaaactttttataatcagtgag

SEQ ID NO: 175 - DNA, 43 bp, Artificial Sequence
  Description of Artificial Sequence Synthetic oligonucleotide
gccaccgagtaaaagaacatcacttgcctgagcgccattaaaa

SEQ ID NO: 176 - DNA, 43 bp, Artificial Sequence
  Description of Artificial Sequence Synthetic oligonucleotide
tctttgattagtaatagtctgtccatcacgcaaattaaccgtt

SEQ ID NO: 177 - DNA, 32 bp, Artificial Sequence
  Description of Artificial Sequence Synthetic oligonucleotide
cgcgtctgataggaacgccatcaacttttaca

SEQ ID NO: 178 - DNA, 32 bp, Artificial Sequence
  Description of Artificial Sequence Synthetic oligonucleotide
aggaagatggggacgacgacagtaatcatatt

SEQ ID NO: 179 - DNA, 32 bp, Artificial Sequence
  Description of Artificial Sequence Synthetic oligonucleotide
ctctagagcaagcttgcatgcctggtcagttg

SEQ ID NO: 180 - DNA, 32 bp, Artificial Sequence
  Description of Artificial Sequence Synthetic oligonucleotide
ccttcaccgtgagacgggcaacagcagtcaca

SEQ ID NO: 181 - DNA, 32 bp, Artificial Sequence
  Description of Artificial Sequence Synthetic oligonucleotide
cgagaaaggaagggaagcgtactatggttgct

SEQ ID NO: 182 - DNA, 40 bp, Artificial Sequence
  Description of Artificial Sequence Synthetic oligonucleotide
gctcattttttaaccagccttcctgtagccaggcatctgc

SEQ ID NO: 183 - DNA, 32 bp, Artificial Sequence
  Description of Artificial Sequence Synthetic oligonucleotide
cagtttgacgcactccagccagctaaacgacg

SEQ ID NO: 184 - DNA, 32 bp, Artificial Sequence
  Description of Artificial Sequence Synthetic oligonucleotide
gccagtgcgatccccgggtaccgagtttttct

SEQ ID NO: 185 - DNA, 40 bp, Artificial Sequence
  Description of Artificial Sequence Synthetic oligonucleotide
tttcaccagcctggccctgagagaaagccggcgaacgtgg

SEQ ID NO: 186 - DNA, 40 bp, Artificial Sequence
  Description of Artificial Sequence Synthetic oligonucleotide
gtaaccgtctttcatcaacattaaaatttttgttaaatca

SEQ ID NO: 187 - DNA, 32 bp, Artificial Sequence
  Description of Artificial Sequence Synthetic oligonucleotide
acgttgtattccggcaccgcttctggcgcatc

SEQ ID NO: 188 - DNA, 32 bp, Artificial Sequence
  Description of Artificial Sequence Synthetic oligonucleotide
ccagggtggctcgaattcgtaatccagtcacg

SEQ ID NO: 189 - DNA, 40 bp, Artificial Sequence
  Description of Artificial Sequence Synthetic oligonucleotide
tagagcttgacggggagttgcagcaagcggtcattgggcg

SEQ ID NO: 190 - DNA, 40 bp, Artificial Sequence
  Description of Artificial Sequence Synthetic oligonucleotide
gttaaaattcgcattaatgtgagcgagtaacacacgttgg

SEQ ID NO: 191 - DNA, 32 bp, Artificial Sequence
  Description of Artificial Sequence Synthetic oligonucleotide
tgtagatgggtgccggaaaccaggaacgccag

SEQ ID NO: 192 - DNA, 32 bp, Artificial Sequence
  Description of Artificial Sequence Synthetic oligonucleotide
ggttttccatggtcatagctgtttgagaggcg

SEQ ID NO: 193 - DNA, 40 bp, Artificial Sequence
  Description of Artificial Sequence Synthetic oligonucleotide
gtttgcgtcacgctggtttgccccaagggagcccccgatt

SEQ ID NO: 194 - DNA, 40 bp, Artificial Sequence
  Description of Artificial Sequence Synthetic oligonucleotide
ggataggtacccgtcggattctcctaaacgttaatatttt

SEQ ID NO: 195 - DNA, 32 bp, Artificial Sequence
  Description of Artificial Sequence Synthetic oligonucleotide
agttgggtcaaagcgccattcgccccgtaatg

SEQ ID NO: 196 - DNA, 32 bp, Artificial Sequence
  Description of Artificial Sequence Synthetic oligonucleotide
cgcgcgggcctgtgtgaaattgttggcgatta

SEQ ID NO: 197 - DNA, 40 bp, Artificial Sequence
  Description of Artificial Sequence Synthetic oligonucleotide
ctaaatcggaaccctaagcaggcgaaaatccttcggccaa

SEQ ID NO: 198 - DNA, 35 bp, Artificial Sequence
  Description of Artificial Sequence Synthetic oligonucleotide
cggcggattgaattcaggctgcgcaacgggggatg

SEQ ID NO: 199 - DNA, 32 bp, Artificial Sequence
  Description of Artificial Sequence Synthetic oligonucleotide
tgctgcaaatccgctcacaattcccagctgca

SEQ ID NO: 200 - DNA, 40 bp, Artificial Sequence
  Description of Artificial Sequence Synthetic oligonucleotide
ttaatgaagtttgatggtggttccgaggtgccgtaaagca

SEQ ID NO: 201 - DNA, 24 bp, Artificial Sequence
  Description of Artificial Sequence Synthetic oligonucleotide
tggcgaaatgttgggaagggcgat

SEQ ID NO: 202 - DNA, 32 bp, Artificial Sequence
  Description of Artificial Sequence Synthetic oligonucleotide
tgtcgtgcacacaacatacgagccacgccagc

SEQ ID NO: 203 - DNA, 40 bp, Artificial Sequence
  Description of Artificial Sequence Synthetic oligonucleotide
caagttttttggggtcgaaatcggcaaaatccgggaaacc

SEQ ID NO: 204 - DNA, 35 bp, Artificial Sequence
  Description of Artificial Sequence Synthetic oligonucleotide
tcttcgctattggaagcataaagtgtatgcccgct

SEQ ID NO: 205 - DNA, 40 bp, Artificial Sequence
  Description of Artificial Sequence Synthetic oligonucleotide
ttccagtccttataaatcaaaagagaaccatcacccaaat

SEQ ID NO: 206 - DNA, 24 bp, Artificial Sequence
  Description of Artificial Sequence Synthetic oligonucleotide
gcgctcacaagcctggggtgccta

SEQ ID NO: 207 - DNA, 40 bp, Artificial Sequence
  Description of Artificial Sequence Synthetic oligonucleotide
cgatggcccactacgtatagcccgagatagggattgcgtt

SEQ ID NO: 208 - DNA, 43 bp, Artificial Sequence
  Description of Artificial Sequence Synthetic oligonucleotide
aactcacattattgagtgttgttccagaaaccgtctatcaggg

SEQ ID NO: 209 - DNA, 43 bp, Artificial Sequence
  Description of Artificial Sequence Synthetic oligonucleotide
acgtggactccaacgtcaaagggcgaatttggaacaagagtcc

SEQ ID NO: 210 - DNA, 25 bp, Artificial Sequence
  Description of Artificial Sequence Synthetic oligonucleotide
ttaattaattttttaccatatcaaa

SEQ ID NO: 211 - DNA, 24 bp, Artificial Sequence
  Description of Artificial Sequence Synthetic oligonucleotide
ttaatttcatcttagactttacaa

SEQ ID NO: 212 - DNA, 23 bp, Artificial Sequence
  Description of Artificial Sequence Synthetic oligonucleotide
ctgtccagacgtataccgaacga

SEQ ID NO: 213 - DNA, 22 bp, Artificial Sequence
  Description of Artificial Sequence Synthetic oligonucleotide
tcaagattagtgtagcaatact

SEQ ID NO: 214 - DNA, 25 bp, Artificial Sequence
  Description of Artificial Sequence Synthetic oligonucleotide
tgtagcattccttttataaacagtt

SEQ ID NO: 215 - DNA, 24 bp, Artificial Sequence
  Description of Artificial Sequence Synthetic oligonucleotide
tttaattgtatttccaccagagcc

SEQ ID NO: 216 - DNA, 23 bp, Artificial Sequence
  Description of Artificial Sequence Synthetic oligonucleotide
actacgaaggcttagcaccatta

SEQ ID NO: 217 - DNA, 22 bp, Artificial Sequence
  Description of Artificial Sequence Synthetic oligonucleotide
ataaggcttgcaacaaagttac

SEQ ID NO: 218 - DNA, 25 bp, Artificial Sequence
  Description of Artificial Sequence Synthetic oligonucleotide
gtgggaacaaatttctatttttgag

SEQ ID NO: 219 - DNA, 24 bp, Artificial Sequence
  Description of Artificial Sequence Synthetic oligonucleotide
cggtgcgggccttccaaaaacatt

SEQ ID NO: 220 - DNA, 23 bp, Artificial Sequence
  Description of Artificial Sequence Synthetic oligonucleotide
atgagtgagcttttaaatatgca

SEQ ID NO: 221 - DNA, 22 bp, Artificial Sequence
  Description of Artificial Sequence Synthetic oligonucleotide
actattaaagaggatagcgtcc

SEQ ID NO: 222 - DNA, 24 bp, Artificial Sequence
  Description of Artificial Sequence Synthetic oligonucleotide
gcgcttaatgcgccgctacagggc

SEQ ID NO: 223 - DNA, 56 bp, Artificial Sequence
  Description of Artificial Sequence Synthetic oligonucleotide
aaaaaaaaaaaaaaaaaaaattttgcgcagagatatcaaaattatttgacattatc

SEQ ID NO: 224 - DNA, 56 bp, Artificial Sequence
  Description of Artificial Sequence Synthetic oligonucleotide
aaaaaaaaaaaaaaaaaaaattttcctgattgaaagaaattgcgtagacccgaacg

SEQ ID NO: 225 - DNA, 56 bp, Artificial Sequence
  Description of Artificial Sequence Synthetic oligonucleotide
aaaaaaaaaaaaaaaaaaaattttattttgcgtctttaggagcactaagcaacagt

SEQ ID NO: 226 - DNA, 56 bp, Artificial Sequence
  Description of Artificial Sequence Synthetic oligonucleotide
aaaaaaaaaaaaaaaaaaaattttaataataaggtcgctgaggcttgcaaagactt

SEQ ID NO: 227 - DNA, 56 bp, Artificial Sequence
  Description of Artificial Sequence Synthetic oligonucleotide
aaaaaaaaaaaaaaaaaaaattttgagaatagcttttgcgggatcgtcgggtagca

SEQ ID NO: 228 - DNA, 56 bp, Artificial Sequence
  Description of Artificial Sequence Synthetic oligonucleotide
aaaaaaaaaaaaaaaaaaaatttttttcatgaaaattgtgtcgaaatctgtacaga

SEQ ID NO: 229 - DNA, 32 bp, Artificial Sequence
  Description of Artificial Sequence Synthetic oligonucleotide
tggtttttaacgtcaaagggcgaagaaccatc

SEQ ID NO: 230 - DNA, 32 bp, Artificial Sequence
  Description of Artificial Sequence Synthetic oligonucleotide
cttgcatgcattaatgaatcggcccgccaggg

SEQ ID NO: 231 - DNA, 32 bp, Artificial Sequence
  Description of Artificial Sequence Synthetic oligonucleotide
tagatggggggtaacgccagggttgtgccaag

SEQ ID NO: 232 - DNA, 32 bp, Artificial Sequence
  Description of Artificial Sequence Synthetic oligonucleotide
catgtcaagattctccgtgggaaccgttggtg

SEQ ID NO: 233 - DNA, 32 bp, Artificial Sequence
  Description of Artificial Sequence Synthetic oligonucleotide
ctgtaatattgcctgagagtctggaaaactag

SEQ ID NO: 234 - DNA, 32 bp, Artificial Sequence
  Description of Artificial Sequence Synthetic oligonucleotide
tgcaactaagcaataaagcctcagttatgacc

SEQ ID NO: 235 - DNA, 32 bp, Artificial Sequence
  Description of Artificial Sequence Synthetic oligonucleotide
aaacagttgatggcttagagcttatttaaata

SEQ ID NO: 236 - DNA, 32 bp, Artificial Sequence
  Description of Artificial Sequence Synthetic oligonucleotide
acgaactagcgtccaatactgcggaatgcttt

SEQ ID NO: 237 - DNA, 32 bp, Artificial Sequence
  Description of Artificial Sequence Synthetic oligonucleotide
ctttgaaaagaactggctcattatttaataaa

SEQ ID NO: 238 - DNA, 32 bp, Artificial Sequence
  Description of Artificial Sequence Synthetic oligonucleotide
acggctacttacttagccggaacgctgaccaa

SEQ ID NO: 239 - DNA, 32 bp, Artificial Sequence
  Description of Artificial Sequence Synthetic oligonucleotide
gagaatagcttttgcgggatcgtcgggtagca

SEQ ID NO: 240 - DNA, 32 bp, Artificial Sequence
  Description of Artificial Sequence Synthetic oligonucleotide
acgttagtaaatgaattttctgtaagcggagt

SEQ ID NO: 241 - DNA, 32 bp, Artificial Sequence
  Description of Artificial Sequence Synthetic oligonucleotide
acccaaatcaagttttttggggtcaaagaacg

SEQ ID NO: 242 - DNA, 32 bp, Artificial Sequence
  Description of Artificial Sequence Synthetic oligonucleotide
tggactcccttttcaccagtgagacctgtcgt

SEQ ID NO: 243 - DNA, 32 bp, Artificial Sequence
  Description of Artificial Sequence Synthetic oligonucleotide
gccagctgcctgcaggtcgactctgcaaggcg

SEQ ID NO: 244 - DNA, 32 bp, Artificial Sequence
  Description of Artificial Sequence Synthetic oligonucleotide
attaagttcgcatcgtaaccgtgcgagtaaca

SEQ ID NO: 245 - DNA, 32 bp, Artificial Sequence
  Description of Artificial Sequence Synthetic oligonucleotide
acccgtcgtcatatgtaccccggtaaaggcta

SEQ ID NO: 246 - DNA, 32 bp, Artificial Sequence
  Description of Artificial Sequence Synthetic oligonucleotide
tcaggtcacttttgcgggagaagcagaattag

SEQ ID NO: 247 - DNA, 32 bp, Artificial Sequence
  Description of Artificial Sequence Synthetic oligonucleotide
caaaattaaagtacggtgtctggaagaggtca

SEQ ID NO: 248 - DNA, 32 bp, Artificial Sequence
  Description of Artificial Sequence Synthetic oligonucleotide
tttttgcgcagaaaacgagaatgaatgtttag

SEQ ID NO: 249 - DNA, 32 bp, Artificial Sequence
  Description of Artificial Sequence Synthetic oligonucleotide
actggataacggaacaacattattaccttatg

SEQ ID NO: 250 - DNA, 32 bp, Artificial Sequence
  Description of Artificial Sequence Synthetic oligonucleotide
cgattttagaggacagatgaacggcgcgacct

SEQ ID NO: 251 - DNA, 32 bp, Artificial Sequence
  Description of Artificial Sequence Synthetic oligonucleotide
gctccatgagaggctttgaggactagggagtt

SEQ ID NO: 252 - DNA, 32 bp, Artificial Sequence
  Description of Artificial Sequence Synthetic oligonucleotide
aaaggccgaaaggaacaactaaagctttccag

SEQ ID NO: 253 - DNA, 32 bp, Artificial Sequence
  Description of Artificial Sequence Synthetic oligonucleotide
agctgattacaagagtccactattgaggtgcc

SEQ ID NO: 254 - DNA, 32 bp, Artificial Sequence
  Description of Artificial Sequence Synthetic oligonucleotide
cccgggtactttccagtcgggaaacgggcaac

SEQ ID NO: 255 - DNA, 32 bp, Artificial Sequence
  Description of Artificial Sequence Synthetic oligonucleotide
gtttgagggaaagggggatgtgctagaggatc

SEQ ID NO: 256 - DNA, 32 bp, Artificial Sequence
  Description of Artificial Sequence Synthetic oligonucleotide
agaaaagcaacattaaatgtgagcatctgcca

SEQ ID NO: 257 - DNA, 32 bp, Artificial Sequence
  Description of Artificial Sequence Synthetic oligonucleotide
caacgcaatttttgagagatctactgataatc

SEQ ID NO: 258 - DNA, 32 bp, Artificial Sequence
  Description of Artificial Sequence Synthetic oligonucleotide
tccatatacatacaggcaaggcaactttattt

SEQ ID NO: 259 - DNA, 32 bp, Artificial Sequence
  Description of Artificial Sequence Synthetic oligonucleotide
caaaaatcattgctccttttgataagtttcat

SEQ ID NO: 260 - DNA, 32 bp, Artificial Sequence
  Description of Artificial Sequence Synthetic oligonucleotide
aaagattcagggggtaatagtaaaccataaat

SEQ ID NO: 261 - DNA, 32 bp, Artificial Sequence
  Description of Artificial Sequence Synthetic oligonucleotide
ccaggcgcttaatcattgtgaattacaggtag

SEQ ID NO: 262 - DNA, 32 bp, Artificial Sequence
  Description of Artificial Sequence Synthetic oligonucleotide
tttcatgaaaattgtgtcgaaatctgtacaga

SEQ ID NO: 263 - DNA, 32 bp, Artificial Sequence
  Description of Artificial Sequence Synthetic oligonucleotide
aataataaggtcgctgaggcttgcaaagactt

SEQ ID NO: 264 - DNA, 32 bp, Artificial Sequence
  Description of Artificial Sequence Synthetic oligonucleotide
cgtaacgatctaaagttttgtcgtgaattgcg

SEQ ID NO: 265 - DNA, 32 bp, Artificial Sequence
  Description of Artificial Sequence Synthetic oligonucleotide
gtaaagcactaaatcggaaccctagttgttcc

SEQ ID NO: 266 - DNA, 32 bp, Artificial Sequence
  Description of Artificial Sequence Synthetic oligonucleotide
agtttggagcccttcaccgcctggttgcgctc

SEQ ID NO: 267 - DNA, 32 bp, Artificial Sequence
  Description of Artificial Sequence Synthetic oligonucleotide
actgcccgccgagctcgaattcgttattacgc

SEQ ID NO: 268 - DNA, 32 bp, Artificial Sequence
  Description of Artificial Sequence Synthetic oligonucleotide
cagctggcggacgacgacagtatcgtagccag

SEQ ID NO: 269 - DNA, 32 bp, Artificial Sequence
  Description of Artificial Sequence Synthetic oligonucleotide
ctttcatccccaaaaacaggaagaccggagag

SEQ ID NO: 270 - DNA, 32 bp, Artificial Sequence
  Description of Artificial Sequence Synthetic oligonucleotide
ggtagctaggataaaaatttttagttaacatc

SEQ ID NO: 271 - DNA, 32 bp, Artificial Sequence
  Description of Artificial Sequence Synthetic oligonucleotide
caataaatacagttgattcccaatttagagag

SEQ ID NO: 272 - DNA, 32 bp, Artificial Sequence
  Description of Artificial Sequence Synthetic oligonucleotide
tacctttaaggtctttaccctgacaaagaagt

SEQ ID NO: 273 - DNA, 32 bp, Artificial Sequence
  Description of Artificial Sequence Synthetic oligonucleotide
tttgccagatcagttgagatttagtggtttaa

SEQ ID NO: 274 - DNA, 32 bp, Artificial Sequence
  Description of Artificial Sequence Synthetic oligonucleotide
tttcaactataggctggctgaccttgtatcat

SEQ ID NO: 275 - DNA, 32 bp, Artificial Sequence
  Description of Artificial Sequence Synthetic oligonucleotide
cgcctgatggaagtttccattaaacataaccg

SEQ ID NO: 276 - DNA, 32 bp, Artificial Sequence
  Description of Artificial Sequence Synthetic oligonucleotide
atatattcttttttcacgttgaaaatagttag

SEQ ID NO: 277 - DNA, 32 bp, Artificial Sequence
  Description of Artificial Sequence Synthetic oligonucleotide
gagttgcacgagatagggttgagtaagggagc

SEQ ID NO: 278 - DNA, 32 bp, Artificial Sequence
  Description of Artificial Sequence Synthetic oligonucleotide
tcatagctactcacattaattgcgccctgaga

SEQ ID NO: 279 - DNA, 32 bp, Artificial Sequence
  Description of Artificial Sequence Synthetic oligonucleotide
gaagatcggtgcgggcctcttcgcaatcatgg

SEQ ID NO: 280 - DNA, 32 bp, Artificial Sequence
  Description of Artificial Sequence Synthetic oligonucleotide
gcaaatatcgcgtctggccttcctggcctcag

SEQ ID NO: 281 - DNA, 32 bp, Artificial Sequence
  Description of Artificial Sequence Synthetic oligonucleotide
tatattttagctgataaattaatgttgtataa

SEQ ID NO: 282 - DNA, 32 bp, Artificial Sequence
  Description of Artificial Sequence Synthetic oligonucleotide
cgagtagaactaatagtagtagcaaaccctca

SEQ ID NO: 283 - DNA, 32 bp, Artificial Sequence
  Description of Artificial Sequence Synthetic oligonucleotide
tcagaagcctccaacaggtcaggatctgcgaa

SEQ ID NO: 284 - DNA, 32 bp, Artificial Sequence
  Description of Artificial Sequence Synthetic oligonucleotide
cattcaacgcgagaggcttttgcatattatag

SEQ ID NO: 285 - DNA, 32 bp, Artificial Sequence
  Description of Artificial Sequence Synthetic oligonucleotide
agtaatcttaaattgggcttgagagaatacca

SEQ ID NO: 286 - DNA, 32 bp, Artificial Sequence
  Description of Artificial Sequence Synthetic oligonucleotide
atacgtaaaagtacaacggagatttcatcaag

SEQ ID NO: 287 - DNA, 32 bp, Artificial Sequence
  Description of Artificial Sequence Synthetic oligonucleotide
aaaaaaggacaaccatcgcccacgcgggtaaa

SEQ ID NO: 288 - DNA, 32 bp, Artificial Sequence
  Description of Artificial Sequence Synthetic oligonucleotide
tgtagcattccacagacagccctcatctccaa

SEQ ID NO: 289 - DNA, 32 bp, Artificial Sequence
  Description of Artificial Sequence Synthetic oligonucleotide
ccccgatttagagcttgacggggaaatcaaaa

SEQ ID NO: 290 - DNA, 32 bp, Artificial Sequence
  Description of Artificial Sequence Synthetic oligonucleotide
gaatagccgcaagcggtccacgctcctaatga

SEQ ID NO: 291 - DNA, 32 bp, Artificial Sequence
  Description of Artificial Sequence Synthetic oligonucleotide
gtgagctagtttcctgtgtgaaatttgggaag

SEQ ID NO: 292 - DNA, 32 bp, Artificial Sequence
  Description of Artificial Sequence Synthetic oligonucleotide
ggcgatcgcactccagccagctttgccatcaa

SEQ ID NO: 293 - DNA, 32 bp, Artificial Sequence
  Description of Artificial Sequence Synthetic oligonucleotide
aaataattttaaattgtaaacgttgatattca

SEQ ID NO: 294 - DNA, 32 bp, Artificial Sequence
  Description of Artificial Sequence Synthetic oligonucleotide
accgttctaaatgcaatgcctgagaggtggca

SEQ ID NO: 295 - DNA, 32 bp, Artificial Sequence
  Description of Artificial Sequence Synthetic oligonucleotide
tcaattcttttagtttgaccattaccagaccg

SEQ ID NO: 296 - DNA, 32 bp, Artificial Sequence
  Description of Artificial Sequence Synthetic oligonucleotide
gaagcaaaaaagcggattgcatcagataaaaa

SEQ ID NO: 297 - DNA, 32 bp, Artificial Sequence
  Description of Artificial Sequence Synthetic oligonucleotide
ccaaaatataatgcagatacataaacaccaga

SEQ ID NO: 298 - DNA, 32 bp, Artificial Sequence
  Description of Artificial Sequence Synthetic oligonucleotide
acgagtagtgacaagaaccggatataccaagc

SEQ ID NO: 299 - DNA, 32 bp, Artificial Sequence
  Description of Artificial Sequence Synthetic oligonucleotide
gcgaaacatgccactacgaaggcatgcgccga

SEQ ID NO: 300 - DNA, 32 bp, Artificial Sequence
  Description of Artificial Sequence Synthetic oligonucleotide
caatgacactccaaaaggagccttacaacgcc

SEQ ID NO: 301 - DNA, 32 bp, Artificial Sequence
  Description of Artificial Sequence Synthetic oligonucleotide
ccagcaggggcaaaatcccttataaagccggc

SEQ ID NO: 302 - DNA, 32 bp, Artificial Sequence
  Description of Artificial Sequence Synthetic oligonucleotide
gctcacaatgtaaagcctggggtgggtttgcc

SEQ ID NO: 303 - DNA, 32 bp, Artificial Sequence
  Description of Artificial Sequence Synthetic oligonucleotide
gcttctggtcaggctgcgcaactgtgttatcc

SEQ ID NO: 304 - DNA, 32 bp, Artificial Sequence
  Description of Artificial Sequence Synthetic oligonucleotide
gttaaaattttaaccaataggaacccggcacc

SEQ ID NO: 305 - DNA, 32 bp, Artificial Sequence
  Description of Artificial Sequence Synthetic oligonucleotide
aggtaaagaaatcaccatcaatataatatttt

SEQ ID NO: 306 - DNA, 32 bp, Artificial Sequence
  Description of Artificial Sequence Synthetic oligonucleotide
tcgcaaatggggcgcgagctgaaataatgtgt

SEQ ID NO: 307 - DNA, 32 bp, Artificial Sequence
  Description of Artificial Sequence Synthetic oligonucleotide
aagaggaacgagcttcaaagcgaagatacatt

SEQ ID NO: 308 - DNA, 32 bp, Artificial Sequence
  Description of Artificial Sequence Synthetic oligonucleotide
ggaattactcgtttaccagacgacaaaagatt

SEQ ID NO: 309 - DNA, 32 bp, Artificial Sequence
  Description of Artificial Sequence Synthetic oligonucleotide
ccaaatcacttgccctgacgagaacgccaaaa

SEQ ID NO: 310 - DNA, 32 bp, Artificial Sequence
  Description of Artificial Sequence Synthetic oligonucleotide
aaacgaaatgacccccagcgattattcattac

SEQ ID NO: 311 - DNA, 32 bp, Artificial Sequence
  Description of Artificial Sequence Synthetic oligonucleotide
tcggtttagcttgataccgatagtccaaccta

SEQ ID NO: 312 - DNA, 32 bp, Artificial Sequence
  Description of Artificial Sequence Synthetic oligonucleotide
tgagtttcgtcaccagtacaaacttaattgta

SEQ ID NO: 313 - DNA, 32 bp, Artificial Sequence
  Description of Artificial Sequence Synthetic oligonucleotide
gaacgtggcgagaaaggaagggaacaaactat

SEQ ID NO: 314 - DNA, 32 bp, Artificial Sequence
  Description of Artificial Sequence Synthetic oligonucleotide
ccgaaatccgaaaatcctgtttgaagccggaa

SEQ ID NO: 315 - DNA, 32 bp, Artificial Sequence
  Description of Artificial Sequence Synthetic oligonucleotide
gcataaagttccacacaacatacgaagcgcca

SEQ ID NO: 316 - DNA, 32 bp, Artificial Sequence
  Description of Artificial Sequence Synthetic oligonucleotide
ttcgccattgccggaaaccaggcattaaatca

SEQ ID NO: 317 - DNA, 32 bp, Artificial Sequence
  Description of Artificial Sequence Synthetic oligonucleotide
gctcattttcgcattaaatttttgagcttaga

SEQ ID NO: 318 - DNA, 32 bp, Artificial Sequence
  Description of Artificial Sequence Synthetic oligonucleotide
agacagtcattcaaaagggtgagaagctatat

SEQ ID NO: 319 - DNA, 32 bp, Artificial Sequence
  Description of Artificial Sequence Synthetic oligonucleotide
tttcatttggtcaataacctgtttatatcgcg

SEQ ID NO: 320 - DNA, 32 bp, Artificial Sequence
  Description of Artificial Sequence Synthetic oligonucleotide
ttttaattgcccgaaagacttcaaaacactat

SEQ ID NO: 321 - DNA, 32 bp, Artificial Sequence
  Description of Artificial Sequence Synthetic oligonucleotide
cataacccgaggcatagtaagagctttttaag

SEQ ID NO: 322 - DNA, 32 bp, Artificial Sequence
  Description of Artificial Sequence Synthetic oligonucleotide
gaataaggacgtaacaaagctgctctaaaaca

SEQ ID NO: 323 - DNA, 32 bp, Artificial Sequence
  Description of Artificial Sequence Synthetic oligonucleotide
ctcatcttgaggcaaaagaatacagtgaattt

SEQ ID NO: 324 - DNA, 32 bp, Artificial Sequence
  Description of Artificial Sequence Synthetic oligonucleotide
cttaaacatcagcttgctttcgagcgtaacac

SEQ ID NO: 325 - DNA, 32 bp, Artificial Sequence
  Description of Artificial Sequence Synthetic oligonucleotide
acgaaccaaaacatcgccattaaatggtggtt

SEQ ID NO: 326 - DNA, 32 bp, Artificial Sequence
  Description of Artificial Sequence Synthetic oligonucleotide
cgacaactaagtattagactttacaataccga

SEQ ID NO: 327 - DNA, 32 bp, Artificial Sequence
  Description of Artificial Sequence Synthetic oligonucleotide
cttttacacagatgaatatacagtaaacaatt

SEQ ID NO: 328 - DNA, 32 bp, Artificial Sequence
  Description of Artificial Sequence Synthetic oligonucleotide
ttaagacgttgaaaacatagcgataacagtac

SEQ ID NO: 329 - DNA, 32 bp, Artificial Sequence
  Description of Artificial Sequence Synthetic oligonucleotide
gcgttatagaaaaagcctgtttagaaggccgg

SEQ ID NO: 330 - DNA, 32 bp, Artificial Sequence
  Description of Artificial Sequence Synthetic oligonucleotide
atcggctgcgagcatgtagaaacctatcatat

SEQ ID NO: 331 - DNA, 32 bp, Artificial Sequence
  Description of Artificial Sequence Synthetic oligonucleotide
cctaatttacgctaacgagcgtctaatcaata

SEQ ID NO: 332 - DNA, 32 bp, Artificial Sequence
  Description of Artificial Sequence Synthetic oligonucleotide
aaaagtaatatcttaccgaagcccttccagag

SEQ ID NO: 333 - DNA, 32 bp, Artificial Sequence
  Description of Artificial Sequence Synthetic oligonucleotide
ttattcatagggaaggtaaatattcattcagt

SEQ ID NO: 334 - DNA, 32 bp, Artificial Sequence
  Description of Artificial Sequence Synthetic oligonucleotide
gagccgccccaccaccggaaccgcgacggaaa

SEQ ID NO: 335 - DNA, 32 bp, Artificial Sequence
  Description of Artificial Sequence Synthetic oligonucleotide
aatgccccgtaacagtgcccgtatctccctca

SEQ ID NO: 336 - DNA, 32 bp, Artificial Sequence
  Description of Artificial Sequence Synthetic oligonucleotide
caagcccaataggaacccatgtacaaacagtt

SEQ ID NO: 337 - DNA, 32 bp, Artificial Sequence
  Description of Artificial Sequence Synthetic oligonucleotide
cggccttgctggtaatatccagaacgaactga

SEQ ID NO: 338 - DNA, 32 bp, Artificial Sequence
  Description of Artificial Sequence Synthetic oligonucleotide
tagccctaccagcagaagataaaaacatttga

SEQ ID NO: 339 - DNA, 32 bp, Artificial Sequence
  Description of Artificial Sequence Synthetic oligonucleotide
ggatttagcgtattaaatcctttgttttcagg

SEQ ID NO: 340 - DNA, 32 bp, Artificial Sequence
  Description of Artificial Sequence Synthetic oligonucleotide
tttaacgttcgggagaaacaataattttccct

SEQ ID NO: 341 - DNA, 32 bp, Artificial Sequence
  Description of Artificial Sequence Synthetic oligonucleotide
tagaatccctgagaagagtcaataggaatcat

SEQ ID NO: 342 - DNA, 32 bp, Artificial Sequence
  Description of Artificial Sequence Synthetic oligonucleotide
aattactacaaattcttaccagtaatcccatc

SEQ ID NO: 343 - DNA, 32 bp, Artificial Sequence
  Description of Artificial Sequence Synthetic oligonucleotide
ctaatttatctttccttatcattcatcctgaa

SEQ ID NO: 344 - DNA, 32 bp, Artificial Sequence
  Description of Artificial Sequence Synthetic oligonucleotide
tcttaccagccagttacaaaataaatgaaata

SEQ ID NO: 345 - DNA, 32 bp, Artificial Sequence
  Description of Artificial Sequence Synthetic oligonucleotide
gcaatagcgcagatagccgaacaattcaaccg

SEQ ID NO: 346 - DNA, 32 bp, Artificial Sequence
  Description of Artificial Sequence Synthetic oligonucleotide
attgagggtaaaggtgaattatcaatcaccgg

SEQ ID NO: 347 - DNA, 32 bp, Artificial Sequence
  Description of Artificial Sequence Synthetic oligonucleotide
aaccagagaccctcagaaccgccaggggtcag

SEQ ID NO: 348 - DNA, 32 bp, Artificial Sequence
  Description of Artificial Sequence Synthetic oligonucleotide
tgccttgactgcctatttcggaacagggatag

SEQ ID NO: 349 - DNA, 32 bp, Artificial Sequence
  Description of Artificial Sequence Synthetic oligonucleotide
aggcggtcattagtctttaatgcgcaatatta

SEQ ID NO: 350 - DNA, 32 bp, Artificial Sequence
  Description of Artificial Sequence Synthetic oligonucleotide
ttattaatgccgtcaatagataatcagaggtg

SEQ ID NO: 351 - DNA, 32 bp, Artificial Sequence
  Description of Artificial Sequence Synthetic oligonucleotide
cctgattgaaagaaattgcgtagacccgaacg

SEQ ID NO: 352 - DNA, 32 bp, Artificial Sequence
  Description of Artificial Sequence Synthetic oligonucleotide
atcaaaatcgtcgctattaattaacggattcg

SEQ ID NO: 353 - DNA, 32 bp, Artificial Sequence
  Description of Artificial Sequence Synthetic oligonucleotide
acgctcaaaataagaataaacaccgtgaattt

SEQ ID NO: 354 - DNA, 32 bp, Artificial Sequence
  Description of Artificial Sequence Synthetic oligonucleotide
ggtattaagaacaagaaaaataattaaagcca

SEQ ID NO: 355 - DNA, 32 bp, Artificial Sequence
  Description of Artificial Sequence Synthetic oligonucleotide
attatttaacccagctacaattttcaagaacg

SEQ ID NO: 356 - DNA, 32 bp, Artificial Sequence
  Description of Artificial Sequence Synthetic oligonucleotide
gaaggaaaataagagcaagaaacaacagccat

SEQ ID NO: 357 - DNA, 32 bp, Artificial Sequence
  Description of Artificial Sequence Synthetic oligonucleotide
gacttgagagacaaaagggcgacaagttacca

SEQ ID NO: 358 - DNA, 32 bp, Artificial Sequence
  Description of Artificial Sequence Synthetic oligonucleotide
gccaccactcttttcataatcaaaccgtcacc

SEQ ID NO: 359 - DNA, 32 bp, Artificial Sequence
  Description of Artificial Sequence Synthetic oligonucleotide
ctgaaacaggtaataagttttaacccctcaga

SEQ ID NO: 360 - DNA, 32 bp, Artificial Sequence
  Description of Artificial Sequence Synthetic oligonucleotide
ctcagagccaccaccctcattttcctattatt

SEQ ID NO: 361 - DNA, 32 bp, Artificial Sequence
  Description of Artificial Sequence Synthetic oligonucleotide
ccgccagccattgcaacaggaaaaatattttt

SEQ ID NO: 362 - DNA, 32 bp, Artificial Sequence
  Description of Artificial Sequence Synthetic oligonucleotide
gaatggctagtattaacaccgcctcaactaat

SEQ ID NO: 363 - DNA, 32 bp, Artificial Sequence
  Description of Artificial Sequence Synthetic oligonucleotide
agattagatttaaaagtttgagtacacgtaaa

SEQ ID NO: 364 - DNA, 32 bp, Artificial Sequence
  Description of Artificial Sequence Synthetic oligonucleotide
acagaaatctttgaataccaagttccttgctt

SEQ ID NO: 365 - DNA, 32 bp, Artificial Sequence
  Description of Artificial Sequence Synthetic oligonucleotide
ctgtaaatcataggtctgagagacgataaata

SEQ ID NO: 366 - DNA, 32 bp, Artificial Sequence
  Description of Artificial Sequence Synthetic oligonucleotide
aggcgttacagtagggcttaattgacaataga

SEQ ID NO: 367 - DNA, 32 bp, Artificial Sequence
  Description of Artificial Sequence Synthetic oligonucleotide
taagtcctaccaagtaccgcactcttagttgc

SEQ ID NO: 368 - DNA, 32 bp, Artificial Sequence
  Description of Artificial Sequence Synthetic oligonucleotide
tattttgctcccaatccaaataagtgagttaa

SEQ ID NO: 369 - DNA, 32 bp, Artificial Sequence
  Description of Artificial Sequence Synthetic oligonucleotide
gcccaataccgaggaaacgcaataggtttacc

SEQ ID NO: 370 - DNA, 32 bp, Artificial Sequence
  Description of Artificial Sequence Synthetic oligonucleotide
agcgccaaccatttgggaattagattattagc

SEQ ID NO: 371 - DNA, 32 bp, Artificial Sequence
  Description of Artificial Sequence Synthetic oligonucleotide
gtttgccacctcagagccgccaccgatacagg

SEQ ID NO: 372 - DNA, 32 bp, Artificial Sequence
  Description of Artificial Sequence Synthetic oligonucleotide
agtgtacttgaaagtattaagaggccgccacc

SEQ ID NO: 373 - DNA, 32 bp, Artificial Sequence
  Description of Artificial Sequence Synthetic oligonucleotide
gccacgctatacgtggcacagacaacgctcat

SEQ ID NO: 374 - DNA, 32 bp, Artificial Sequence
  Description of Artificial Sequence Synthetic oligonucleotide
attttgcgtctttaggagcactaagcaacagt

SEQ ID NO: 375 - DNA, 32 bp, Artificial Sequence
  Description of Artificial Sequence Synthetic oligonucleotide
gcgcagagatatcaaaattatttgacattatc

SEQ ID NO: 376 - DNA, 32 bp, Artificial Sequence
  Description of Artificial Sequence Synthetic oligonucleotide
taacctccatatgtgagtgaataaacaaaatc

SEQ ID NO: 377 - DNA, 32 bp, Artificial Sequence
  Description of Artificial Sequence Synthetic oligonucleotide
catatttagaaataccgaccgtgttacctttt

SEQ ID NO: 378 - DNA, 32 bp, Artificial Sequence
  Description of Artificial Sequence Synthetic oligonucleotide
caagcaagacgcgcctgtttatcaagaatcgc

SEQ ID NO: 379 - DNA, 32 bp, Artificial Sequence
  Description of Artificial Sequence Synthetic oligonucleotide
ttttgtttaagccttaaatcaagaatcgagaa

SEQ ID NO: 380 - DNA, 32 bp, Artificial Sequence
  Description of Artificial Sequence Synthetic oligonucleotide
atacccaagataacccacaagaataaacgatt

SEQ ID NO: 381 - DNA, 32 bp, Artificial Sequence
  Description of Artificial Sequence Synthetic oligonucleotide
aatcaccaaatagaaaattcatatataacgga

SEQ ID NO: 382 - DNA, 32 bp, Artificial Sequence
  Description of Artificial Sequence Synthetic oligonucleotide
caccagagttcggtcatagcccccgccagcaa

SEQ ID NO: 383 - DNA, 32 bp, Artificial Sequence
  Description of Artificial Sequence Synthetic oligonucleotide
cctcaagaatacatggcttttgatagaaccac

SEQ ID NO: 384 - DNA, 32 bp, Artificial Sequence
  Description of Artificial Sequence Synthetic oligonucleotide
ccctcagaaccgccaccctcagaactgagact

SEQ ID NO: 385 - DNA, 32 bp, Artificial Sequence
  Description of Artificial Sequence Synthetic oligonucleotide
ggaaatacctacattttgacgctcacctgaaa

SEQ ID NO: 386 - DNA, 32 bp, Artificial Sequence
  Description of Artificial Sequence Synthetic oligonucleotide
gcgtaagagagagccagcagcaaaaaggttat

SEQ ID NO: 387 - DNA, 32 bp, Artificial Sequence
  Description of Artificial Sequence Synthetic oligonucleotide
ctaaaatagaacaaagaaaccaccagggttag

SEQ ID NO: 388 - DNA, 32 bp, Artificial Sequence
  Description of Artificial Sequence Synthetic oligonucleotide
aacctaccgcgaattattcatttccagtacat

SEQ ID NO: 389 - DNA, 32 bp, Artificial Sequence
  Description of Artificial Sequence Synthetic oligonucleotide
aaatcaatggcttaggttgggttactaaattt

SEQ ID NO: 390 - DNA, 32 bp, Artificial Sequence
  Description of Artificial Sequence Synthetic oligonucleotide
aatggtttacaacgccaacatgtagttcagct

SEQ ID NO: 391 - DNA, 32 bp, Artificial Sequence
  Description of Artificial Sequence Synthetic oligonucleotide
aatgcagaccgtttttattttcatcttgcggg

SEQ ID NO: 392 - DNA, 32 bp, Artificial Sequence
  Description of Artificial Sequence Synthetic oligonucleotide
aggttttgaacgtcaaaaatgaaagcgctaat

SEQ ID NO: 393 - DNA, 32 bp, Artificial Sequence
  Description of Artificial Sequence Synthetic oligonucleotide
atcagagaaagaactggcatgattttattttg

SEQ ID NO: 394 - DNA, 32 bp, Artificial Sequence
  Description of Artificial Sequence Synthetic oligonucleotide
tcacaatcgtagcaccattaccatcgttttca

SEQ ID NO: 395 - DNA, 32 bp, Artificial Sequence
  Description of Artificial Sequence Synthetic oligonucleotide
tcggcattccgccgccagcattgacgttccag

SEQ ID NO: 396 - DNA, 32 bp, Artificial Sequence
  Description of Artificial Sequence Synthetic oligonucleotide
taagcgtcgaaggattaggattagtaccgcca

SEQ ID NO: 397 - DNA, 32 bp, Artificial Sequence
  Description of Artificial Sequence Synthetic oligonucleotide
ctaaagcaagatagaacccttctgaatcgtct

SEQ ID NO: 398 - DNA, 32 bp, Artificial Sequence
  Description of Artificial Sequence Synthetic oligonucleotide
cggaattattgaaaggaattgaggtgaaaaat

SEQ ID NO: 399 - DNA, 32 bp, Artificial Sequence
  Description of Artificial Sequence Synthetic oligonucleotide
gagcaaaaacttctgaataatggaagaaggag

SEQ ID NO: 400 - DNA, 32 bp, Artificial Sequence
  Description of Artificial Sequence Synthetic oligonucleotide
tatgtaaaccttttttaatggaaaaattacct

SEQ ID NO: 401 - DNA, 32 bp, Artificial Sequence
  Description of Artificial Sequence Synthetic oligonucleotide
agaggcataatttcatcttctgactataacta

SEQ ID NO: 402 - DNA, 32 bp, Artificial Sequence
  Description of Artificial Sequence Synthetic oligonucleotide
tcattacccgacaataaacaacatatttaggc

SEQ ID NO: 403 - DNA, 32 bp, Artificial Sequence
  Description of Artificial Sequence Synthetic oligonucleotide
ctttacagttagcgaacctcccgacgtaggaa

SEQ ID NO: 404 - DNA, 32 bp, Artificial Sequence
  Description of Artificial Sequence Synthetic oligonucleotide
ttattacggtcagagggtaattgaatagcagc

SEQ ID NO: 405 - DNA, 32 bp, Artificial Sequence
  Description of Artificial Sequence Synthetic oligonucleotide
ccggaaacacaccacggaataagtaagactcc

SEQ ID NO: 406 - DNA, 32 bp, Artificial Sequence
  Description of Artificial Sequence Synthetic oligonucleotide
tgaggcaggcgtcagactgtagcgtagcaagg

SEQ ID NO: 407 - DNA, 32 bp, Artificial Sequence
  Description of Artificial Sequence Synthetic oligonucleotide
tgctcagtcagtctctgaatttaccaggaggt

SEQ ID NO: 408 - DNA, 32 bp, Artificial Sequence
  Description of Artificial Sequence Synthetic oligonucleotide
tatcaccgtactcaggaggtttagcggggttt

SEQ ID NO: 409 - DNA, 32 bp, Artificial Sequence
  Description of Artificial Sequence Synthetic oligonucleotide
gaaatggattatttacattggcagacattctg

SEQ ID NO: 410 - DNA, 32 bp, Artificial Sequence
  Description of Artificial Sequence Synthetic oligonucleotide
gccaacagtcaccttgctgaacctgttggcaa

SEQ ID NO: 411 - DNA, 32 bp, Artificial Sequence
  Description of Artificial Sequence Synthetic oligonucleotide
atcaacagtcatcatattcctgattgattgtt

SEQ ID NO: 412 - DNA, 32 bp, Artificial Sequence
  Description of Artificial Sequence Synthetic oligonucleotide
tggattatgaagatgatgaaacaaaatttcat

SEQ ID NO: 413 - DNA, 32 bp, Artificial Sequence
  Description of Artificial Sequence Synthetic oligonucleotide
ttgaattatgctgatgcaaatccacaaatata

SEQ ID NO: 414 - DNA, 32 bp, Artificial Sequence
  Description of Artificial Sequence Synthetic oligonucleotide
ttttagtttttcgagccagtaataaattctgt

SEQ ID NO: 415 - DNA, 32 bp, Artificial Sequence
  Description of Artificial Sequence Synthetic oligonucleotide
ccagacgagcgcccaatagcaagcaagaacgc

SEQ ID NO: 416 - DNA, 32 bp, Artificial Sequence
  Description of Artificial Sequence Synthetic oligonucleotide
gaggcgttagagaataacataaaagaacaccc

SEQ ID NO: 417 - DNA, 32 bp, Artificial Sequence
  Description of Artificial Sequence Synthetic oligonucleotide
tgaacaaacagtatgttagcaaactaaaagaa

SEQ ID NO: 418 - DNA, 32 bp, Artificial Sequence
  Description of Artificial Sequence Synthetic oligonucleotide
acgcaaaggtcaccaatgaaaccaatcaagtt

SEQ ID NO: 419 - DNA, 32 bp, Artificial Sequence
  Description of Artificial Sequence Synthetic oligonucleotide
tgcctttagtcagacgattggcctgccagaat

SEQ ID NO: 420 - DNA, 32 bp, Artificial Sequence
  Description of Artificial Sequence Synthetic oligonucleotide
ggaaagcgaccaggcggataagtgaataggtg

SEQ ID NO: 421 - DNA, 27 bp, Artificial Sequence
  Description of Artificial Sequence Synthetic oligonucleotide
aacatcacttgcctgagtagaagaact

SEQ ID NO: 422 - DNA, 27 bp, Artificial Sequence
  Description of Artificial Sequence Synthetic oligonucleotide
tgtagcaatacttctttgattagtaat

SEQ ID NO: 423 - DNA, 27 bp, Artificial Sequence
  Description of Artificial Sequence Synthetic oligonucleotide
agtctgtccatcacgcaaattaaccgt

SEQ ID NO: 424 - DNA, 27 bp, Artificial Sequence
  Description of Artificial Sequence Synthetic oligonucleotide
ataatcagtgaggccaccgagtaaaag

SEQ ID NO: 425 - DNA, 26 bp, Artificial Sequence
  Description of Artificial Sequence Synthetic oligonucleotide
acgccagaatcctgagaagtgttttt

SEQ ID NO: 426 - DNA, 26 bp, Artificial Sequence
  Description of Artificial Sequence Synthetic oligonucleotide
ttaaagggattttagacaggaacggt

SEQ ID NO: 427 - DNA, 26 bp, Artificial Sequence
  Description of Artificial Sequence Synthetic oligonucleotide
agagcgggagctaaacaggaggccga

SEQ ID NO: 428 - DNA, 26 bp, Artificial Sequence
  Description of Artificial Sequence Synthetic oligonucleotide
tataacgtgctttcctcgttagaatc

SEQ ID NO: 429 - DNA, 26 bp, Artificial Sequence
  Description of Artificial Sequence Synthetic oligonucleotide
gtactatggttgctttgacgagcacg

SEQ ID NO: 430 - DNA, 26 bp, Artificial Sequence
  Description of Artificial Sequence Synthetic oligonucleotide
gcgcttaatgcgccgctacagggcgc